(19) United States
(12) Patent Application Publication
(10) Pub. No.: US 2008/0207669 A1
(43) Pub. Date: Aug. 28, 2008

(54) (S)-N-Stereoisomers of 7,8-Saturated-4,5-Epoxy-Morphinanium Analogs

(75) Inventors: Julio Perez, Tarrytown, NY (US); Amy Qi Han, Hockessin, DE (US); Yakov Rotshteyn, Monroe, NY (US)

Correspondence Address: KELLEY DRYE & WARREN LLP, 400 ALTLANTIC STREET , 13TH FLOOR, STAMFORD, CT, 06901, US

(73) Assignee: Progenics Pharmaceuticals, Inc., Tarrytown, NY (US)

(21) Appl. No.: 11/944,242

(22) Filed: Nov. 21, 2007

Related U.S. Application Data
(60) Provisional application No. 60/867,101, filed on Nov. 22, 2006.
(60) Provisional application No. 60/867,394, filed on Nov. 27, 2006.

(51) Int. Cl. A61K 31/485; C07D 489/08; A61P 1/12; A61P 1/00

(52) U.S. Cl. 514/282; 546/45

(57) ABSTRACT

Novel (S)-N-stereoisomers of 7,8-saturated-4,5-epoxy-morphinanium analogs are disclosed. Pharmaceutical compositions containing the (S)-N-stereoisomers of 7,8-saturated-4,5-epoxy-morphinanium analogs and methods for their pharmaceutical uses are also disclosed. Such analogs are disclosed as being useful in treating, among varying conditions, hypermotility of the gastrointestinal tract.

### BACKGROUND OF THE INVENTION

1. Field of the Invention

The present invention generally relates to (S)-7,8-N-single-bond-4,5-epoxy-morphinanium analogs (hereinafter referenced to as “7,8-saturated-4,5-epoxy-morphinaniums”), including 7,8-saturated-4,5-epoxy-morphinanium analogs, synthetic methods for their preparation, pharmaceutical preparations comprising the same, and methods for their use. This application claims priority to U.S. application 60/867,101, filed Nov. 22, 2006 and U.S. application 60/867,394, filed Nov. 27, 2006, each of which is hereby incorporated in entirety.

2. Description of the Related Art

The medicinal and psychological effects of opium have been known since ancient times. It was not, however, until around the beginning of the nineteenth century, that morphine was isolated from opium, and codeine and papaverine thereafter. By the middle of the nineteenth century pure alkaloids rather than crude opium preparations were becoming established medical practice. Since the nineteenth century a host of synthetic and semi-synthetic derivatives of these natural alkaloids have been made.

In respect of morphinan compounds, it is now known that substituent substitutions can have significant effects on the pharmacology. For example, some have reported that the 3-hydroxy morphinans may be significantly less effective orally than parenterally possibly due to a significant first-pass metabolism. Glucuronidation of morphine at its 3-hydroxyl group is believed to terminate the activity. However, 3-methoxy groups such as seen in oxycodone and codeine have been associated by some with good oral potency.

methyl group in morphine and related opioids by substituents rich in π-electrons, such as allyl, cylcobutylmethyl, and propylmethyl, result in potent antagonists such as nalorphine, naloxone, naltrexone and nalbuphine.

The designations “R” and “S” are commonly used in organic chemistry to denote specific configuration of a chiral center. The designations “R” refers to “right” and refers to that configuration of a chiral center with a clockwise relationship of group priorities (highest to second lowest) when viewed along the bond toward the lowest priority group. The term “S” or “left” refers to that configuration of chiral center with a along the bond toward the lowest priority group.

The priority of groups for the R/S designation is based upon atomic number (heaviest isotope first). A partial list of priorities and a discussion of stereochemistry is contained in the book: The Vocabulary of Organic Chemistry, Orchin, et al. John Wiley and Sons, Inc., page 126 (1980), which is incorporated herein by reference in its entirety. When quaternary nitrogen morphinan structures are produced, such structures may be characterized as (R) or (S) stereoisomers.

The art suggests that isolated stereoisomers of a compound, whether enantiomers or diastereomers, sometimes may have contrasting physical and functional properties, although it is unpredictable whether this is the case in any particular circumstance. Dextromethorphan is a cough suppressant, whereas its enantiomer, levomethorphan, is a potent narcotic. (R,R)-methylphenidate is a drug to treat attention deficit hyperactivity disorder (ADHD), whereas its enantiomer, (S,S)-methylphenidate is an antidepressant. (S)-fluoxetine is active against migraine, whereas its enantiomer, (R)-fluoxetine is used to treat depression. The (S)-enantiomer of citalopram is therapeutically active isomer for treatment of depression. The (R)-enantiomer is inactive. The (S)-enantiomer of omeprazole is more potent for the treatment of heartburn than the (R) enantiomer.

Caldwell et al., Complete Proton and Carbon Nuclear Magnetic Resonance Spectral Assignments of Some Morphin-6-one Alkaloids by Two-Dimensional NMR Techniques, describe the use of two-dimensional NMR conformational analysis (Nuclear Overhauser enhancement difference analysis) with respect to select quaternary N-methyl oxycodone analogs to determine that the N-methyl group was in the equatorial position. They noted that proton coupling constants with respect to the compounds they tested suggested that the cyclohexanone ring and piperidine rings of the morphinan backbone adopt slightly distorted chair conformations.

Bianchetti et al., Quaternary Derivatives of Narcotic Antagonists: Stereochemical Requirements at the Chiral Nitrogen for In Vitro and In Vivo Activity, 1983 Life Science 33 (Sup 1):415-418 studied three pairs of diastereoisomers of quaternary narcotic antagonist and their parent tertiary amines, levallorphan, nalorphine, and naloxone, to see how the configuration about the chiral nitrogen affected in vitro and in vivo activity. It was found that the activity varied considerably depending on how the quaternary derivatives were prepared. In each series, only the diastereomer obtained by methylation of the N-allyl-substituted tertiary amine (referred to as “N-methyl diastereomer”) was potent in displacing ³H-naltrexone from rat brain membranes, and acting as a morphine antagonist in the guinea-pig ileum. Conversely, diastereoisomers obtained by reacting N-methyl-substituted tertiary amines with allyl halide (referred to as “N-allyl diastereomers”) did not displace 3H-naltrexone and had negligible antagonist activity and slight agonist action in the guinea-pig ileum. In vivo findings were generally consistent with those in vitro. Thus only the “N-methyl” but not the “N-allyl diastereomers” inhibited morphine-induced constipation in rats and behaved as antagonists. The author stated that the prepared materials appeared to be pure by ¹H and ¹³C nuclear magnetic resonance (NMR) analysis, but these methods are not accurate. The author cites a literature reference for the assignment of the (R) configuration to the “N-methyl diastereomer” of nalorphine. No assignment is proposed for the levallorphan and naloxone diastereomers. It would be adventurous to extrapolate the configuration to these diastereomers (R. J. Kobylecki et al, J. Med. Chem. 25, 1278-1280, 1982).

Kobylecki et al., 1982, N-Methylnalorphine: Definition of N-allyl conformation for antagonism at the opiate receptor, J. Med. Chem. 25:1278-1280 report, based on X-ray diffraction data, that the active diastereomer derived from nalorphine (N-methyl diastereomer) the allyl group about the quaternary nitrogen has an equatorial configuration. Kobylecki reported that the isomer with the axial N-substituent demonstrated some agonist activity (although very low) with very substantial antagonist activity in comparison (to its agonist activity) whereas the equatorial N-substituent displayed pure opioid antagonist activity.

Iorio et al., Narcotic agonist/antagonist properties of quaternary diasteromers derived from oxymorphone and naloxone, 1984, Clim. Ther. 19: 301-303, indicates that correlations between agonist and antagonist ratio and N-substitution orientation follow the same pattern found by Kobylecki with respect to diastereoisomeric quaternary morphinanium salts, that is, that compounds with larger groups equatorially displayed more antagonist activity than the corresponding axial diastereoisomer. These authors suggest that all types of activity, agonism, antagonism and mixed activity, may all be explained by different conformational types of interaction of equatorial N-substituents with receptor subsites. Comparison of activity of the compounds they produced was by direct in vitro ileum contraction tests, and in vivo by injecting the compounds into the brain of mice. Funke and deGraaf, A ¹H and ¹³C nuclear magnetic resonance study of three quaternary salts of naloxone and oxymorphone, 1986, J. Chem. Soc. Perkin Trans. II 735-738, referencing Ioria et al., report the ¹H and ¹³C n.m.r. data with three N,N-dialkyl-morphinanium chloride derivates (one N,N-diallyl and two N-allyl-N-methyl diastereoisomers).

Cooper (U.S. Pat. No. 6,455,537) disputes the relevancy of the Iorio in vivo data arguing that the administration into the brain was not appropriate given that quaternized agents do not pass into the brain. Cooper performing a number of in vivo tests using intravenous methylnalorphine, found that the (R)-isomer of N-methylnalorphine provided superior treatment to antagonize or prevent opiate induced side effects in mammals such as nausea, vomiting and ataxia, when compared with the (S)-isomer or a mixture of R/S N-methylnalorphine.

Feinberg et al., The opiate receptor: A model explaining structure-activity relationships of opiate agonists and antagonists, 1976 Proc. Natl. Acad. Sci. USA 73: 4215-4219, opine that the spatial location of “antagonist substituents” such as N-allyl and cyclopropylmethyl, determine the “purity” of the antagonistic pharmacological properties of an opioid drug. Feinberg et al. theorize that a 14-hydroxyl group on the morphinan structure helps to increase the proportion of antagonistic substituents in the equatorial conformation relative to axial conformation in respect of the piperidine ring, that such equatorial confirmation at least with respect to N-allyl and cyclopropylmethyl increase the “pure” antagonism. They further theorize that in mediating antagonist activity that the specific antagonist binding site of the receptor interacts with the pi-electrons of the N-allyl or the atomic configurations to N-cyclopropylmethyl or N-cyclobutylmethyl groups, which are required for antagonist pharmacology, thus stabilizing antagonist receptor conformation. To secure “pure” antagonist properties, they suggest that the approximation of the antagonist substituent to the antagonist binding site of the receptor must be facilitated by a 14-hydroxyl or 9-β-methyl substituent as seen in naloxone or benzomorphan antagonists. Without such substituents, they hypothesize varying mixtures of agonist and antagonist pharmacology.

While such references may suggest improved antagonistic activity for certain functional groups on a morphinan nitrogen when such groups are in an equatorial position, in conjunction they do not suggest the agonist-antagonist activity of isolated (R), (S) conformers or axial-equatorial conformers for morphinan compounds with different substituents, particularly with respect to compounds supporting different saturation profiles in respect of the rings of the backbone morphinan structure, compounds carrying a quaternary charged nitrogen, and compounds with different substituent pairs at the 3 and 6 positions of the morphinan backbone.

### SUMMARY OF THE INVENTION

Disclosed in embodiments described herein are (S)-7,8-saturated-4,5-epoxy-morphinanium analogs which have been produced in high purity, permitting the characterization of their relative retention time in chromatography versus that of their corresponding (R)-saturated-4,5-epoxy-morphinanium analog. The diastereomers of such analogs have been found to have activity different from that of their corresponding diastereomeric mixtures.

In an embodiment of the present invention, there is provided substantially or highly pure (S)-7,8-saturated-4,5-epoxy-morphinanium, crystals of substantially of highly pure (S)-7,8-saturated-4,5-epoxy-morphinanium and intermediates thereof, novel methods for making substantially or highly pure (S)-7,8-saturated-4,5-epoxy-morphinanium compounds, methods for analyzing, quantitating and isolating (S)-7,8-saturated-4,5-epoxy-morphinanium compounds in a mixture containing counterpart (R)-7,8-saturated-4,5-epoxy-morphinanium stereoisomer and particular (S)-7,8-saturated-4,5-epoxy-morphinaniums, methods of distinguishing an (R)-7,8-saturated-4,5-epoxy-morphinanium from its (S)-7,8-saturated-4,5-epoxy-morphinanium stereoisomer, pharmaceutical products containing the same and related uses of these materials.

Salts of (S)-7,8-saturated-4,5-epoxy-morphinaniums are also provided. A protocol for obtaining (S)-7,8-saturated-4,5-epoxy-morphinaniums is also provided. In addition, it has been discovered, surprisingly, that (S)-7,8-saturated-4,5-epoxy-morphinaniums have opioid agonist activity. The invention provides synthetic routes for stereoselective synthesis of (S)-7,8-saturated-4,5-epoxy-morphinaniums, substantially pure (S)-7,8-saturated-4,5-epoxy-morphinaniums, crystals of substantially pure (S)-7,8-saturated-4,5-epoxy-morphinaniums, pharmaceutical preparations containing substantially pure (S)-7,8-saturated-4,5-epoxy-morphinaniums, and methods for their use.

According to one embodiment of the invention, a composition is provided that comprises a 7,8-saturated-4,5-epoxy-morphinanium in the (S) configuration (that is, with respect to the nitrogen) is present at greater than 99.5%. In other embodiments the 7,8-saturated-4,5-epoxy-morphinanium in (S)-configuration (with respect to the nitrogen) is present in the composition in greater than about 99.6%, or about 99.7%, or about 99.8%, or about 99.9%, or about 99.95%, or even more preferably greater than 99.95%. In one embodiment, there is no detectable counterpart (R)-7,8-saturated-4,5-epoxy-morphinanium compound in the analyzed composition using the chromatographic procedures described herein. It may be preferred that the composition is free of the corresponding (R)-7,8-saturated-4,5-epoxy-morphinanium as detected on HPLC. In one embodiment, there is no HPLC detectable counterpart (R)-7,8-saturated-4,5-epoxy-morphinanium at a detection limit of 0.02% and a quantitation limit of 0.05%. In yet another embodiment the composition of the invention contains 99.85% of the 7,8-saturated-4,5-epoxy-morphinanium in the (S)-configuration with respect to nitrogen, and it contains the counterpart stereoisomeric (R)-7,8-saturated-4,5-epoxy-morphinanium compound at a HPLC detectable detection limit of 0.02% and a quantitation limit of 0.05%.

According to one aspect of the invention, a composition is provided that comprises a 7,8-saturated-4,5-epoxy-morphinanium, wherein at least 99.6%, 99.7%, 99.8%, 99.85%, 99.9%, and even 99.95% of the 7,8-saturated-4,5-epoxy-morphinanium compound in the composition is in the (S)-configuration with respect to nitrogen, and the composition includes one or more of: a buffering agent, a chelating agent, a preserving agent, a cryoprotecting agent, a permeation enhancer, a lubricating agent, a preservative, an anti-oxidant, or a binding agent.

(S)-7,8-saturated-4,5-epoxy-morphinaniums of the present invention include the structure of Formula Z:

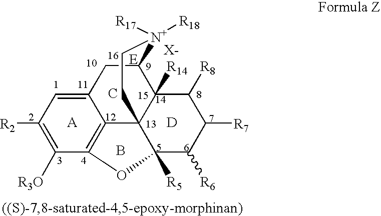

wherein X is a counterion and the compound is an (S) configuration about the nitrogen in conformity with the Cahn, Ingold, Prelog configuration assignment rules, and R₁₈ and R₁₇ are C₁-C₈ alkyls, or C₁-C₆ alkyls. R₃ may be a hydroxyl protecting group. The molecule can exist as a zwitterion. The counterion can be any counterion. Preferably the anion is pharmaceutically acceptable. Anions include halides, sulfates, phosphates, nitrates, and anionic-charged organic species. The halide can be iodide, bromide, chloride, fluoride, or combinations thereof. In one embodiment the halide is iodide. In one embodiment, the halide is bromide. The anionic-charged organic species may be a sulfonate or carboxylate.

An aspect of the invention is directed to an isolated compound of the (S) configuration with respect to the nitrogen of formula I:

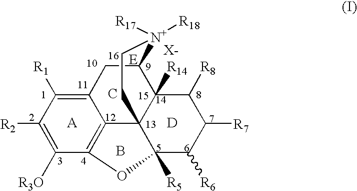

or a pharmaceutically acceptable salt form or prodrug form thereof, wherein:
R₁ and R₂ are independently H, OH, OR₂₆, halide, silyl; hydrocarbyl, cyclohydrocarbyl, or substituted moieties thereof, or R₁ and R₂, can also be combined to form a C₃-C₆ carbocycle fused ring which may be substituted according to R₁₉, a benzo fused ring, or a 5-6 membered heteroaryl fused ring;
R₃ is H, silyl;
   (C₁-C₉) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
   C₁-C₃ acyl
R₅ is H, CH, OR₂₆,
   (C₁-C₈) alkyl substituted with 0-3 R₁₉;
   (C₂-C₈) alkenyl substituted with 0-3 R₁₉;
   (C₂-C₈) alkynyl substituted with 0-3 R₁;
   (C₃-C₁₀) cycloalkyl substituted with 0-3R₂₀;
   (C₃-C₁₀) carbocycle substituted with 0-3R₂₀;
   aryl substituted with 0-3R₂₀;
R₆ is H, ═O, OH, OR₂₆;
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
   amine, amide, sulfonamide, or ester;
R₇ and R₅ are independently H, hydrocarbyl, cyclohydrocarbyl, or substituted moieties thereof; or R₇ and R₈ are combined to form a carbocycle fused ring which may be substituted according to R₁₉, a benzo fused ring, or a 5-6 membered heteroaryl fused ring;
R₁₄ is H, OH, OR₂₆, NR₂₂R₂₃SR₂₅, S(═O)R₂₅, SO₂R₂₅;
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₃;
   (C₃-C₁₀) carbocycle substituted with 0-3R₂₀;
   aryl substituted with 0-3R₂₀; aryloxy, acyloxy,
   or R₁₄ can be combined with R₁₇ or R₁₈ depending on its configuration with respect to quaternary nitrogen to form an O-fused ring, or a C₃-C₆ carbocycle fused ring;
R₁₇ and R₁₈ are C₁-C₆ hydrocarbyls which may be substituted, wherein if R₁₈ is methyl, R₁₇ is not allyl;
R₁₉ is at each occurrence is independently selected from:
   H, C₁-C₆ alkyl, CF₃, OR₂₄, Cl, F, Br, I, ═O, CN, NO₂, NR₂₂R₂₃;
   C₃-C₁₀ carbocycle substituted with 0-3 R₂₁;
   aryl substituted with 0-3 R₂₁; or
   5 to 10 membered heterocycle containing 1 to 4 heteroatoms selected from nitrogen, oxygen, and sulphur, wherein said 5 to 10 membered heterocycle is substituted with 0-3 R₂₁;
R₂₀ at each occurrence, is independently selected from H, OH, Cl, F, Br, I, CN, NO₂, NR₂₂R₂₃, acetyl,
   C₁-C₆ alkyl, C₁-C₄ alkoxy, C₁-C₄ haloalkyl,
   C₁-C₄ haloalkoxy, and C₁-C₄ haloalkyl-S—;
R₂₁, at each occurrence, is independently selected from H, OH, Cl, F, Br, I, CN, NO₂, NR₂₂R₂₃, CF₃, acetyl,
   C₁-C₆ alkyl, C₁-C₄ alkoxy, C₁-C₄ haloalkyl,
   C₁-C₄ haloalkoxy, and C₁-C₄ haloalkyl-S—; or
   NR₂₂R₂₃ may be a heterocyclic ring selected from the group piperidinyl, homopiperidinyl, thiomorpholinyl, piperizinyl, and morpholinyl;
R₂₂, at each occurrence, is independently selected from H, C₁-C₆ alkyl, (C₁-C₆ alkyl)-C(═O)—, and (C₁-C₆ alkyl)-S(═O)₂—;
R₂₃, at each occurrence, is independently selected from:
   H, (C₁-C₆)alkyl,
   (C₁-C₆ alkyl)-C(O)—, and (C₁-C₆ alkyl)-S(═O)₂—;
R₂₄, at each occurrence, is independently selected from H, phenyl, benzyl, (C₁-C₆) alkyl, and (C₂-C₆) alkoxyalkyl;
R₂₅ is alkyl, aryl, or arylalkyl;
R₂₆ is at each occurrence is independently selected from
   H, C₁-C₆ alkyl, CF₃;
   C₃-C₁₀ carbocycle substituted with 0-3 R₂₁;
   aryl substituted with 0-3 R₂₁; or
   5 to 10 membered heterocycle containing 1 to 4 heteroatoms selected from nitrogen, oxygen, and sulphur, wherein said 5 to 10 membered heterocycle is substituted with 0-3 R₂₁; and
X⁻ is an anion.

Included in embodiments herein are the S-stereoisomers with respect to the nitrogen of formula Ia:

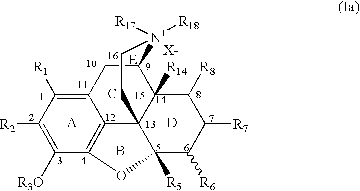

wherein
R₁₇ and R₁₈ are selected alternatively with respect to one another from (a) or (b):
   (a) unsubstituted or non-halogen substituted: C₄-C₈ (cycloalkyl)alkyl or (cycloalkenyl)alkyl, (cycloheteryl)alkyl, (cycloaryl)alkyl; C₄-C₆ (cycloalkyl)alkyl or (cycloalkenyl)alkyl (cycloheteryl)alkyl, (cycloaryl)alkyl
   (b) substituted or unsubstituted linear or branched C₁-C₃ alkyl, C₂-C₃ alkenyl, or C₃-alkynyl;
   wherein if (b) is selected as methyl, and R₆ is ═O, (a) is not unsubstituted (cyclopropyl)methyl;
R₆ is H, OH, ═O, ═CH₂, —N(CH₃)₂, or any cyclic ring, or forms a cyclic ring with R₇;
R₇ and R₈ are H or alkyl;
R₁₄ is H, OH, halide, arylamido, amino, N-alkyl, N-dialkyl, N-aryl, N-alkylaryl, N-cycloalkylalkyl, SCH₃, S(═O)CH₃, S(═O)₂CH₃, alkoxy, aryloxy, or aryl-alkoxy or forms a cyclic ring with R₁₇ or R₁₈;
R₁ and R₂ are independently H, halide, alkoxy, alkyl, or aryl;
R₃ is H, C₁-C₄ alkyl, or C₁-C₃ acyl, -silyl;
R₅ is H, OH, alkyl, alkoxy, or aryloxy; and
X⁻ is an anion.

Included in embodiments herein are the (S)-stereoisomers about the nitrogen of the formula Ib:

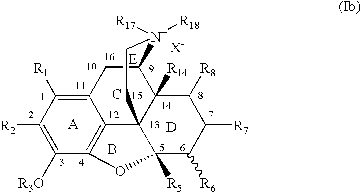

wherein
R₁₇ and R₁₈ are a substituted or unsubstituted C₁-C₆ hydrocarbyl, wherein when R is selected as ═O, at least one of which is not methyl when the other is cyclopropylmethyl;
R₆ is H, OH, OR₂₅, ═O, ═CH₂, —N-alkyl, N-dialkyl, acyloxy, alkoxy, alkyl, ═CR′R″ where R′ and R″ are independently H or C₁-C₁₀ alkyl, or any ring, or R₆ forms a ring with R₇;
R₇ and R₈ are H or hydrocarbyl, cyclohydrocarbyl, alkoxy, amine, amide, hydroxy or substituted moieties thereof;
R₁₄ is H, OH, halide, N-alkyl, N-dialkyl, N-aryl, N-alkylaryl, N-cycloalkylalkyl, SR₂₅, S(═O)R₂₅, SO₂R₂₅; alkoxy, aryloxy, or arylalkoxy, or forms a ring with R₁₇ or R₁₈;
R₁ and R₂ are independently H, halide, alkoxy, alkyl, or aryl;
R₃ is H, alkyl, C₁-C₃ acyl, silyl;
R₅ is H, OH, alkyl, alkoxy, or aryloxy;
R₂₅ is alkyl, aryl, arylalkyl; and
X⁻ is an anion.

Certain groups may be preferentially chosen. For example, R₁₄ may be selected to be OH or O-alkyl in one embodiment.

An isolated compound of the (S) configuration with respect to the nitrogen of Formula I(c):

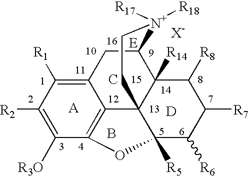

or a pharmaceutically acceptable salt form or prodrug form thereof, wherein:
R₁ and R₂ are independently H, OH, OR₂₆, halide, silyl; hydrocarbyl, cyclohydrocarbyl, or substituted moieties thereof;
   or R₁ and R₂ can also be combined to form a C₃-C₆ carbocycle fused ring which may be substituted according to R₁₉, a benzo fused ring, or a 5-6 membered heteroaryl fused ring;
R₃ is H, silyl, CO₂R₁₉, SO₂R₁₉, B(OR₂₆)₂;
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
   C₁-C₃ acyl
R₅ is H, OH, OR₂₆,
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
R₆ is H, ═O, OH, OR₂₆, ═(R₁₉)(R_{19′}), =(hetero cycle substituted with 0-3R₂₆), ═(C₃-C₇ cycle substituted with 0-3R₂₀);
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
   amine, amide, sulfonamide, or ester;
R₇ and R₈ are independently H, hydrocarbyl, cyclohydrocarbyl, hetero cycle with 0-3R₂₀, alkylaryl with 0-3R₂₀, arylakly with 0-3 R₂₀, or substituted moieties thereof, or

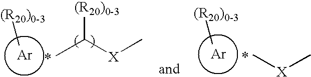

where, X is bond, ═O, O, S, N(R₁₉), SO, SO₂, SO₂N(R₁₉), CON(R₁₉), N(R₁₉)CON(R_{19′}), N(R₁₉)C(═NR_{19′})N(R_{19″}), COO;
or R₇ and R₈ are combined to form a carbocycle fused ring which may be substituted according to R₁₉, a benzo fused ring, 5-, 6-, or a 5-6 membered aryl or heteroaryl with 0-3R₁₀;
R₁₄ is H, OH, OR₂₆, NR₂₂R₂₃SR₂₅, S(═O)R₂₅, SO₂R₂₅, hetero cycle with 0-3R₂₀, alkylaryl with 0-3R₂₀, arylalkyl with 0-3R₂₀,

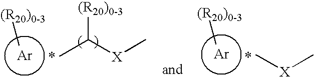

wherein, X is bond, ═O, O, S, N(R₁₉), SO, SO₂, SO₂N(R₁₉), CON(R₁₉), N(R₁₉)CON(R_{19′}), N(R₁₉)C(═NR_{19′})N(R_{19″}), COO;
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀; aryloxy, acyloxy,
or R₁₄ can be combined with R₁₈ depending on its configuration with respect to quaternary nitrogen to form an O-fused ring, or a C₃-C₆ carbocycle fused ring;
R₁₇ and R₁₈ are C₁-C₆ hydrocarbyls which may be substituted, wherein if R₁₈ is methyl, R₁₇ is not allyl, hetero cycle with 0-3R₂₀, alkylaryl with 0-3R₂₀, arylalkyl with 0-3_{R20},

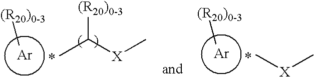

wherein, X is bond, ═O, O, S, N(R₁₉), SO, SO₂, SO₂N(R₁₉), CON(R₁₉), N(R₁₉)CON(R_{19′}), N(R₁₉)C(═NR_{19′})N(R_{19″}), COO;
R₁₉ is at each occurrence is independently selected from: H, C₁-C₆ alkyl, CF₃, OR₂₄, Cl, F, Br, I, ═O, CN, NO₂, NR₂₂R₂₃, aryl substituted with 0-3R₂₀;
   C₃-C₁₀ carbocycle substituted with 0-3 R₂₁; aryl substituted with 0-3 R₂₀; or
   5 to 10 membered heterocycle containing 1 to 4 heteroatoms selected from nitrogen, oxygen, and sulphur, wherein said 5 to 10 membered heterocycle is substituted with 0-3 R₂₁;
R₂₀ at each occurrence, is independently selected from H, OH, Cl, F, Br, I, CN, NO₂, NR₂₂R₂₃, acetyl, OR₂₅, XR₂₅,
   C₁-C₆ alkyl, C₁-C₄ alkoxy, C₁-C₄ haloalkyl,
   C₁-C₄ haloalkoxy, and C₁-C₄ haloalkyl-S—;
R₂₁, at each occurrence, is independently selected from H, OH, Cl, F, Br, I, CN, NO₂, NR₂₂R₂₃, CF₃, acetyl, OR₂₅, XR₂₅,
   C₁-C₆ alkyl, C₁-C₄ alkoxy, C₁-C₄ haloalkyl,
   C₁-C₄ haloalkoxy, and C₁-C₄ haloalkyl-S—; or
   NR₂₂R₂₃ may be a heterocyclic ring selected from the group piperidinyl, homopiperidinyl, thiomorpholinyl, piperizinyl, and morpholinyl;
R₂₂, at each occurrence, is independently selected from H, C₁-C₆ alkyl, C₆-C₁₀ aryl, hetero aryl, hetero cycle, alkylaryl, and arylalkyl;
   (C₁-C₆ alkyl)-C(═O)—, and (C₁-C₆ alkyl)-S(═O)₂—;
R₂₃, at each occurrence, is independently selected from: H, (C₁-C₆)alkyl, C₆-C₁₀ aryl, hetero aryl, hetero cycle, alkylaryl, haloalkyl, arylalkyl,
   (C₁-C₆ alkyl)-C(═O)—, and (C₁-C₆ alkyl)-S(═O)₂—;
R₂₄, at each occurrence, is independently selected from H, phenyl, benzyl, (C₁-C₆) alkyl, and (C₂-C₆) alkoxyalkyl;
R₂₅ is alkyl, aryl, or arylalkyl;
R₂₆ is at each occurrence is independently selected from:
   H, C₁-C₆ alkyl, CF₃;
   C₃-C₁₀ carbocycle substituted with 0-3 R₂₁;
   aryl substituted with 0-3 R₂; or
   5 to 10 membered heterocycle containing 1 to 4 heteroatoms selected from nitrogen, oxygen, and sulphur, wherein said 5 to 10 membered heterocycle is substituted with 0-3 R₂₁; and
X⁻ is an anion.

(S)-7,8-saturated-4,5-epoxy-morphinanium, as illustrated, are salts. Therefore, there will be an anion, which for the present application includes a halide, sulfate, phosphate, nitrate, or anionic-charged organic species. Halides include fluoride, chloride, iodide and bromide. In some embodiments, the halide is iodide and in other embodiments, the halide is bromide. In some embodiments the anionic-charged species is a sulfonate or a carboxylate. Examples of sulfonates include mesylate, besylate, tosylate, and triflate. Examples of carboxylates include formate, acetate, citrate, and fumarate.

According to another aspect of the invention, the foregoing compositions that comprise in a (S)-configuration with respect to nitrogen in some embodiments is a crystal, a solution, or a bromide salt of a 7,8-saturated-4,5-epoxy-morphinanium. In other embodiments, the foregoing compositions are pharmaceutical preparations, preferably in effective amounts and with a pharmaceutically acceptable carrier.

According to one aspect of the invention, a crystal of a certain 7,8-saturated-4,5-epoxy-morphinanium is provided that is at least about 99.5%, or about 99.6% or about 99.7%, or is about 99.8%, or about 99.9%, or most preferably greater than 99.95% of the 7,8-saturated-4,5-epoxy-morphinanium in (S)-configuration with respect to the nitrogen.

According to another embodiment of the invention, an (S)-7,8-saturated-4,5-epoxy-morphinanium compound is provided in isolated form. By isolated, it is meant at least 50% pure. In embodiments, (S)-7,8-saturated-4,5-epoxy-morphinanium is provided at 75% purity, at 90% purity, at 95% purity, at 98% purity, and even at 99% purity or above. In an embodiment, the (S)-7,8-saturated-4,5-epoxy-morphinanium is in a crystal form.

According to another aspect of the invention, a composition is provided. The composition comprises a 7,8-saturated-4,5-epoxy-morphinanium, wherein the 7,8-saturated-4,5-epoxy-morphinanium present in the composition is greater than 10% in (S) configuration with respect to nitrogen. More preferably, the 7,8-saturated-4,5-epoxy-morphinanium present in the composition is greater than 30%, 40%, 50%, 60%, 70%, 75%, 80%, 85%, 90%, 95%, 96%, 97%, 98%, 98.5%, 99%, 99.5%, 99.6%, 99.7%, 99.8%, and even 99.9% in (S) configuration with respect to nitrogen. In some embodiments there is no detectable counterpart (R)-7,8-saturated-4,5-epoxy-morphinanium compound as measured by high performance liquid chromatography (HPLC).

The composition in some embodiments is a solution, in others an oil, in others a cream, and in still others a solid or semi-solid. In one embodiment, the composition is a crystal.

According to another aspect of the invention, a pharmaceutical preparation is provided. The pharmaceutical preparation includes any one of the compositions of a particular (S)-7,8-saturated-4,5-epoxy-morphinanium described above in a pharmaceutically acceptable carrier. The pharmaceutical preparation contains a effective amount of the (S)-7,8-saturated-4,5-epoxy-morphinanium. In some embodiments, there is little or no detectable counterpart (R)-7,8-saturated-4,5-epoxy-morphinanium structure in the composition. If present, (R)-7,8-saturated-4,5-epoxy-morphinanium compound is at a level such that effective amounts of the (S)-7,8-saturated-4,5-epoxy-morphinanium compound are administered to a subject. In some embodiments, the pharmaceutical preparation further includes a therapeutic agent other than the 7,8-saturated-4,5-epoxy-morphinanium. In one embodiment, the therapeutic agent is an opioid or opioid agonist. Examples of opioids or opioid agonists are alfentanil, anileridine, asunadoline, bremazocine, burprenorphine, butorphanol, codeine, dezocine, diacetylmorphine (heroin), saturatedcodeine, diphenoxylate, fedotozine, fentanyl, funaltrexamine, hydrocodone, hydromorphone, levallorphan, levomethadyl acetate, levorphanol, loperamide, meperidine (pethidine), methadone, morphine, morphine-6-glucuronide, nalbuphine, nalorphine, opium, oxycodone, oxymorphone, pentazocine, propiram, propoxyphene, remifentanyl, sufentanil, tilidine, trimebutine, tramadol, or combinations thereof. In some embodiments, the opioid or opioid agonist does not readily cross the blood brain barrier and, therefore, has substantially no central nervous system (CNS) activity when administered systemically (i.e., it is of the class of agents known as “peripherally acting”) agents.

In other embodiments the therapeutic agent is an opioid antagonist. Opioid antagonists include peripheral mu opioid antagonists. Examples of peripheral mu opioid antagonists include quaternary derivatives of noroxymorphone (See Goldberg et al, U.S. Pat. No. 4,176,186, and Cantrell et al WO 2004/043964), piperidine N-alkylcarboxylates such as described in U.S. Pat. Nos. 5,250,542; 5,434,171; 5,159,081; 5,270,328; and 6,469,030, opium alkaloid derivatives such as described in U.S. Pat. Nos. 4,730,048; 4,806,556; and 6,469,030, quaternary benzomorphan compounds such as described in U.S. Pat. Nos. 3,723,440 and 6,469,030. In one embodiment, the peripheral opioid antagonist is an (S)-7,8-saturated-4,5-epoxy-morphinanium.

In other embodiments, the therapeutic agent is not an opioid, opioid agonist, or an opioid antagonist. For example, the therapeutic agent can be an antiviral agent, antibiotic agent, antifungal agent, antibacterial agent, antiseptic agent, anti-protozoal agent, anti-parasitic agent, anti-inflammatory agent, a vasoconstrictor agent, a local anesthetic agent, an anti-diarrheal agent, an anti-hyperalgesia agent, or combinations thereof.

In one embodiment of the invention, the (S)-7,8-saturated-4,5-epoxy-morphinanium is combined with an anti-diarrhea agent that is loperamide, loperamide analogs, N-oxides of loperamide and analogs, metabolites and prodrugs thereof, diphenoxylate, cisapride, antacids, aluminum hydroxide, magnesium aluminum silicate, magnesium carbonate, magnesium hydroxide, calcium carbonate, polycarbophil, simethicone, hyoscyamine, atropine, furazolidone, difenoxin, octreotide, lansoprazole, kaolin, pectin, activated charcoal, sulphaguanidine, succinylsulphathiazole, phthalylsulphathiazole, bismuth aluminate, bismuth subcarbonate, bismuth subcitrate, bismuth citrate, tripotassium dicitrato bismuthate, bismuth tartrate, bismuth subsalicylate, bismuth subnitrate and bismuth subgallate, opium tincture (paregoric), herbal medicines, plant-derived anti-diarrheal agents or combinations thereof.

In one aspect of the invention, the (S)-7,8-saturated-4,5-epoxy-morphinanium is combined with an anti-inflammatory agent that is a non-steroidal anti-inflammatory drug (NSAID), a tumor necrosis factor inhibitor, basiliximab, daclizumab, infliximab, mycophenolate, mofetil, azothioprine, tacrolimus, steroids, sulfasalazine, olsalazine, mesalamine, or combinations thereof.

The pharmaceutical preparations of the invention can take on a variety of forms, including, but not limited to a composition that is enteric coated, a composition that is an immediate release formulation, a controlled release or sustained release formulation, a composition that is a solution, a composition that is a topical formulation, a composition that is a suppository, a composition that is lyophilized, a composition that is in an inhaler, a composition that is in a nasal spray device, and the like. The composition can be for oral administration, parenteral administration, mucosal administration, nasal administration, topical administration, ocular administration, local administration, etc. If parenteral, the administration can be subcutaneous, intravenous, intradermal, intraperitoneal, intrathecal, etc.

According to another embodiment of the invention, a method for synthesizing (S)-7,8-saturated-4,5-epoxy-morphinanium analog salts is provided. The method involves combining an alkylhalide (e.g., an iodomethyl cyclopropane if a methylcyclopropane moiety is desired to be added to the nitrogen) structure (for example, noroxymorphone if a noroxymorphone derivative is desired) in a first solvent to produce a halide salt of (S)-7,8-saturated-4,5-epoxy-morphinanium. Counterions then may be substituted, optionally, for example, an iodide may be exchanged by transferring the iodo salt (S)-7,8-saturated-4,5-epoxy-morphinanium to a second solvent and exchanging iodide for a counterion other than iodide. For example, the iodo salt of (S)-7,8-saturated-4,5-epoxy-morphinanium may be transferred from a first solvent to a second solvent, and the iodide exchanged in the second solvent for bromide to produce a bromo salt of (S)-7,8-saturated-4,5-epoxy-morphinanium. The first solvent may be, e.g., a dipolar aprotic solvent. The first solvent may be, for example, N-methylpyrrolidone (NMP) or DMF. The second solvent may be, for example, methylene chloride isopropyl acetate, dioxane.

Certain embodiments entail purification of the salt of the (S)-7,8-saturated-4,5-epoxy-morphinanium by chromatography, recrystallization, or a combination thereof. In one embodiment, the purification is by multiple recrystallizations.

In some embodiments, the reaction may be carried out across a wide temperature spectrum and at atmospheric conditions. In other embodiments, the reaction in the first solvent may need to be conducted under a controlled reaction temperature, for example, between 65° to 75° C., or at about 70° C., and the reaction in the second solvent may be conducted at another temperature, for example at room temperature.

The method overall may involve synthesizing (S)-7,8-saturated-4,5-epoxy-morphinanium analogs plus counterion by combining the appropriate derivative with an appropriate tertiary oxymorphan in a first solvent to produce the (S)-analog plus counterion. The appropriate derivative may contain a leaving group, such as a halide or sulfonate. The halide may be, for example, iodide. The first solvent may be a dipolar aprotic solvent. Examples of such solvents are N-methylpyrrolidone, dimethyl formamide, methylphosphoramide, acetone, 1,4-dioxane, and acetonitrile and combinations thereof. Preferred is N-methylpyrrolidone. The first solvent may alternatively be a dipolar protic solvent. Examples are 2-propanol, 1-propanol, ethanol, methanol. The method can further involve exchanging the counterion of the formed (S)-7,8-saturated-4,5-epoxy-morphinanium with another counterion. Examples of counterions are bromide, chloride, fluoride, nitrate, sulfonate, or carboxylate. The sulfonate can be mesylate, besylate, tosylate or triflate. The carboxylate can be formate, acetate, citrate and fumarate. The method can involve transferring the (S)-7,8-saturated-4,5-epoxy-morphinanium counterion to a second solvent prior to exchanging the counterion of (S)-7,8-saturated-4,5-epoxy-morphinanium with another counterion. The method can further involve purifying the (S)-7,8-saturated-4,5-epoxy-morphinanium plus counterion, for example by recrystallization, by chromatography or by both.

According to another aspect of the invention, method is provided for inhibiting diarrhea in a subject, by administering to a subject in need of such treatment a pharmaceutical composition containing (S)-7,8-saturated-4,5-epoxy-morphinanium in an amount effective to treat or prevent the diarrhea. The pharmaceutical preparation can be of the type described above. The diarrhea can be acute or chronic. The diarrhea can be caused by any variety of circumstances, alone or combined, such as caused by an infectious agent, food intolerance, food allergy, malabsorption syndrome, reaction to a medication or nonspecific etiology. In some embodiments, the diarrhea is associated with irritable bowel disease or with inflammatory bowel disease. In one embodiment the inflammatory bowel disease is celiac disease. In another embodiment the inflammatory bowel disease is Crohn's disease. In yet another embodiment, the inflammatory bowel disease is ulcerative colitis. In other embodiments the diarrhea results from stomach or bowel resection, removal of a gall bladder, or organic lesions. In other embodiments, the diarrhea is associated with a carcinoid tumor or vasoactive intestinal polypeptide-secreting tumor. In still other embodiments, the diarrhea is chronic functional (idiopathic) diarrhea.

According to the invention, the (S)-7,8-saturated-4,5-epoxy-morphinanium may be administered in conjunction with an anti-diarrheal agent that is not (S)-7,8-saturated-4,5-epoxy-morphinanium. By in conjunction with, it is meant at the same time or close enough in time whereby both agents are treating the condition at the same time. In one embodiment, the agent is an opioid or an opioid agonist. In another embodiment, the agent is not an opioid or an opioid agonist.

According to another aspect of the invention, a method is provided for reducing a volume of discharge from a ileostomy or colostomy in a subject. The method involves administering to a subject in need of such reduction a pharmaceutical composition containing an (S)-7,8-saturated-4,5-epoxy-morphinanium in an amount effective to reduce the volume of discharge from the ileostomy or colostomy. The pharmaceutical preparation can be of the type described above.

According to another aspect of the invention, a method is provided for reducing a rate of discharge from a ileostomy or colostomy in a subject. The method involves administering to a subject in need of such reduction a pharmaceutical composition containing an (S)-7,8-saturated-4,5-epoxy-morphinanium in an amount effective to reduce the rate of discharge from the ileostomy or colostomy. The pharmaceutical preparation can be of the type described above.

According to another aspect of the invention, a method is provided for inhibiting gastrointestinal motility in a subject. The method involves administering to a subject in need of such inhibition a pharmaceutical composition containing an (S)-7,8-saturated-4,5-epoxy-morphinanium of the present disclosure in an amount effective to inhibit gastrointestinal motility in the subject. The pharmaceutical preparation can be of the type described above. According to the invention, the (S)-7,8-saturated-4,5-epoxy-morphinanium may be administered in conjunction with another motility inhibiting agent that is not a (S)-7,8-saturated-4,5-epoxy-morphinanium. In one embodiment, the agent is an opioid or an opioid agonist. Opioids and opioid agonists are described above. In another embodiment, the agent is not an opioid or an opioid agonist. Examples of such gastrointestinal motility inhibiting agents are described below, each as if recited specifically in this summary of invention.

According to another aspect of the invention, a method is provided for treating irritable bowel syndrome. The method involves administering to a patient in need of such treatment a pharmaceutical composition containing an (S)-7,8-saturated-4,5-epoxy-morphinanium of the present disclosure in an amount effective to ameliorate at least one symptom of the irritable bowel syndrome. The pharmaceutical preparation can be of the type described above. In one embodiment, the symptom is diarrhea. In another embodiment, the symptom is alternating constipation and diarrhea. In another embodiment, the symptom is abdominal pain, abdominal bloating, abnormal stool frequency, abnormal stool consistency, or combinations thereof.

According to another aspect of the invention, a method is provided for inhibiting pain in a subject. The pain may be acute pain or chronic pain. The method involves administering to a patient in need of such treatment a pharmaceutical composition containing an (S)-7,8-saturated-4,5-epoxy-morphinanium in an amount effective to inhibit the pain. The pharmaceutical preparation can be of the type described above. The method can further involve administering to the subject a therapeutic agent other than (S)-7,8-saturated-4,5-epoxy-morphinanium. In one embodiment, the agent other than (S)-7,8-saturated-4,5-epoxy-morphinanium is an opioid. In another embodiment, the agent other than (S)-7,8-saturated-4,5-epoxy-morphinanium is a nonopioid pain relieving agent. Nonopioid pain relieving agents include corticosteroids and nonsteroidal anti-inflammatory drugs. Pain relieving agents are described in greater detail below, as if recited herein this summary. If the pain is peripheral hyperalgesia, it can result, for example, from a bite, sting, burn, viral or bacterial infection, oral surgery, tooth extraction, injury to the skin and flesh, wound, abrasion, contusion, surgical incision, sunburn, rash, skin ulcers, mucositis, gingivitis, bronchitis, laryngitis, sore throat, shingles, fungal irritation, fever blisters, boils, plantar's warts, vaginal lesions, anal lesions, corneal abrasion, post-radial keratectomy, or inflammation. It also can be associated with post-surgery recovery. The surgery can be, for example, radial keratectomy, tooth extraction, lumpectomy, episiotomy, laparoscopy, and arthroscopy. In another embodiment, the agent other than (S)-7,8-saturated-4,5-epoxy-morphinanium is an antiviral agent, antibiotic agent, antifungal agent, antibacterial agent, antiseptic agent, anti-protozoal agent, anti-parasitic agent, anti-inflammatory agent, a vasoconstrictor agent, a local anesthetic agent, an anti-diarrheal agent, or an anti-hyperalgesia agent.

In some embodiments, the pharmaceutical composition is administered locally to a site of the pain. In some embodiments, the administration is intra-articular. In some embodiments, the administration is systemic. In some embodiments, the administration is topical. In some embodiments, the composition is administered to the eye.

According to another aspect of the invention, a method is provided for inhibiting inflammation in a subject. The method involves administering to a patient in need of such treatment a pharmaceutical composition containing an (S)-7,8-saturated-4,5-epoxy-morphinanium in an amount effective to inhibit the inflammation. The pharmaceutical preparation can be of the type described above. The method can also involve administering to the subject a therapeutic agent other than an (S)-7,8-saturated-4,5-epoxy-morphinanium. The therapeutic agent other than an (S)-7,8-saturated-4,5-epoxy-morphinanium can be an anti-inflammatory agent. The administration can be, for example, local administration at a site of the inflammation, systemic administration, or topical administration.

The inflammation in some embodiments is periodontal inflammation, orthodontic inflammation, inflammatory conjunctivitis, hemorrhoids and venereal inflammations. In other embodiments, the inflammation is a skin inflammatory condition. Examples include inflammation associated with a disorder selected from the group consisting of irritant contact dermatitis, psoriasis, eczema, pruritus, seborrheic dermatitis, nummular dermatitis, lichen planus, acne vulgaris, comedones, polymorphs, nodulokystic acne, conglobata, senile acne, secondary acne, medical acne, a keratlmization disorder, and blistery derma, atopic dermatitis, and UV-induced inflammation. The skin inflammatory condition also can be associated with skin sensitization or irritation arising from the use of a cosmetic or skin care product which causes skin sensitization or irritation or can be a non-allergic inflammatory skin condition. It also can be induced by all-tran(S)-retinoic acid. In other embodiments, the inflammation can be a systemic inflammatory condition. Examples include conditions selected from the group consisting of inflammatory bowel disease, rheumatoid arthritis, cachexia, asthma, Crohn's disease, endotoxin shock, adult respiratory distress syndrome, ischemic/reperfusion damage, graft-versus-host reactions, bone resorption, transplantation and lupus. Other embodiments can involve inflammation associated with a condition selected from the group consisting of multiple sclerosis, diabetes, and wasting associated with acquired immunodeficiency syndrome (AIDS) or cancer.

According to another aspect of the invention, a method is provided for inhibiting the production of tumor necrosis factor in a subject. The method involves administering to a patient in need of such treatment a pharmaceutical composition containing an (S)-7,8-saturated-4,5-epoxy-morphinanium in an amount effective to inhibit the production of tumor necrosis factor. The pharmaceutical preparation can be of the type described above. The method can also involve administering to the subject a therapeutic agent other than an (S)-7,8-saturated-4,5-epoxy-morphinanium.

According to another embodiment of the invention, a method is provided for regulating gastrointestinal function in a subject. The method involves administering to a patient in need of such treatment a pharmaceutical composition containing an (S)-7,8-saturated-4,5-epoxy-morphinanium and administering to the subject a peripheral mu opioid antagonist, both in amounts to regulate gastrointestinal function. In one embodiment, the peripheral mu opioid antagonist is an (R)-7,8-saturated-4,5-epoxy-morphinanium.

According to another embodiment of the invention, a method is provided. The method involves preventing or treating a psychogenic eating or digestive disorder by administering to a patient a composition described above in an amount effective to prevent or treat the psychogenic eating or digestive disorder.

According to another embodiment of the invention, a kit is provided. The kit includes a package containing a sealed container of a pharmaceutical composition containing an (S)-7,8-saturated-4,5-epoxy-morphinanium. The kit further can include a therapeutic agent other than an (S)-7,8-saturated-4,5-epoxy-morphinanium. The therapeutic agent other than the (S)-7,8-saturated-4,5-epoxy-morphinanium in one embodiment is an opioid or opioid agonist. In one aspect, the opioid or opioid agonist has substantially no CNS activity when administered systemically (i.e., is “peripherally acting”). In other embodiments, the therapeutic agent other than the (S)-7,8-saturated-4,5-epoxy-morphinanium is an opioid antagonist. Opioid antagonists include peripheral mu opioid antagonists. In one embodiment, the peripheral opioid antagonist is an (R)-7,8-saturated-4,5-epoxy-morphinanium. In other embodiments, the agent other than the (S)-7,8-saturated-4,5-epoxy-morphinanium is an antiviral agent, antibiotic agent, antifungal agent, antibacterial agent, antiseptic agent, anti-protozoal agent, anti-parasitic agent, anti-inflammatory agent, a vasoconstrictor agent, a local anesthetic agent, an anti-diarrheal agent, or an anti-hyperalgesia agent, or combinations thereof.

According to an embodiment of the invention, a method for analyzing an (S)-7,8-saturated-4,5-epoxy-morphinanium in a mixture of an (R)-7,8-saturated-4,5-epoxy-morphinanium and its isostereomeric counterpart is provided. The method involves conducting high performance liquid chromatography (HPLC) and applying (S)-7,8-saturated-4,5-epoxy-morphinanium to the chromatography column as a standard. The method preferably involves applying both the (S)-7,8-saturated-4,5-epoxy-morphinanium and its stereoisomeric counterpart (R)-7,8-saturated-4,5-epoxy-morphinanium as standards to determine relative retention/elution times. Relative retention times of the (R) and (S)-7,8-saturated-4,5-epoxy-morphinanium are disclosed therein. In one embodiment, the chromatography is conducted using two solvents, solvent A and solvent B, wherein, for example, solvent A is an aqueous solvent and solvent B is a methanolic solvent and wherein, for example, both A and B contain trifluoroacetic acid (TFA), for example, A being 0.1% aqueous TFA and B being 0.1% methanolic TFA. In embodiments, the column comprises a bonded, end-capped silica. In embodiments, the pore size of the column gel is 5 microns. In one embodiment, the column, flow rate and gradient program are as follows:

Column: Luna C18(2), 150 × 4.6 mm, 5μ
Flow Rate: 1 mL/min
Gradient Program:

| Time (min) | % A | % B |
|---|---|---|
| 0:00 | 95 | 5 |
| 8:00 | 65 | 35 |
| 12:00 | 35 | 65 |
| 15:00 | 0 | 100 |
| 16:00 | 95 | 5 |
| 18:00 | 95 | 5 |

Detection can be carried out conveniently by ultraviolet (UV)@230 nm wavelength.
The foregoing HPLC also can be used to determine the relative amount of an (S)-7,8-saturated-4,5-epoxy-morphinanium and its counterpart stereoisomer (R)-7,8-saturated-4,5-epoxy-morphinanium by determining the area under the respective (R) and (S) curves in the chromatogram produced.

According to another embodiment of the invention, methods are provided for ensuring the manufacture of (S)-7,8-saturated-4,5-epoxy-morphinanium (which is an opioid agonist) that is free of (R)-7,8-saturated-4,5-epoxy-morphinanium (which is an opioid antagonist). The methods permit for the first time the assurance that a pharmaceutical preparation of the (S)-7,8-saturated-4,5-epoxy-morphinaniums of the present disclosure which are intended for agonist activity are not contaminated with a compound that opposes the activity of the (S)-7,8-saturated-4,5-epoxy-morphinanium (i.e., its (R)-7,8-saturated-4,5-epoxy-morphinanium stereoisomer). In this aspect of the invention, a method is provided for manufacturing (S)-7,8-saturated-4,5-epoxy-morphinanium. The method involves: (a) obtaining a first composition containing (S)-7,8-saturated-4,5-epoxy-morphinanium of interest, (b) purifying the first composition by chromatography, recrystallization or a combination thereof, (c) conducting HPLC on a sample of purified first composition using the counterpart (R)-7,8-saturated-4,5-epoxy-morphinanium as a standard, and (d) determining the presence or absence of the counterpart (R)-7,8-saturated-4,5-epoxy-morphinanium in the sample. In some embodiments, both an (R)-7,8-saturated-4,5-epoxy-morphinanium and its (S)-7,8-saturated-4,5-epoxy-morphinanium stereoisomer are used as standards to determine, for example, relative retention time of the (R)-7,8-saturated-4,5-epoxy-morphinanium and (S)-7,8-saturated-4,5-epoxy-morphinanium. In one embodiment, the purifying is multiple recryallization steps or multiple chromatography steps. In another embodiment, the purifying is carried out until the (R)-7,8-saturated-4,5-epoxy-morphinanium stereoisomer is absent from the sample as determined by HPLC. It should be understood, however, that the “purified first composition” in some aspects of the invention is not necessarily free of the detectable (R)-7,8-saturated-4,5-epoxy-morphinanium. The presence of such (R)-7,8-saturated-4,5-epoxy-morphinanium, for example, might indicate that further purification steps should be conducted if pure (S)-7,8-saturated-4,5-epoxy-morphinanium is desired. The methods can further involve packaging purified first composition that is free of HPLC detectable (R)-7,8-saturated-4,5-epoxy-morphinanium. The methods further can include providing indicia on or within the packaged, purified first composition indicating that the packaged, purified first composition is free of HPLC detectable (R)-7,8-saturated-4,5-epoxy-morphinanium. The method further can involve packaging a pharmaceutically effective amount for treating anyone of the conditions described herein. The first composition containing the (S)-7,8-saturated-4,5-epoxy-morphinanium can be obtained by the methods described herein. Pure (R)-7,8-saturated-4,5-epoxy-morphinanium counterpart can be obtained as described herein.

According to another embodiment of the invention, a packaged product is provided. The package contains a composition comprising the (S)-7,8-saturated-4,5-epoxy-morphinanium, wherein the composition is free of HPLC detectable (R)-7,8-saturated-4,5-epoxy-morphinanium counterpart, and indicia on or contained within the package indicating that the composition is free of detectable (R)-7,8-saturated-4,5-epoxy-morphinanium stereoisomer. The composition can take on a variety of forms, including, but not limited to, a standard for use in laboratory experiments, a standard for use in manufacturing protocols, or a pharmaceutical composition. If the composition is a pharmaceutical composition, then one form of indicia is writing on a label or package insert describing the characteristics of the pharmaceutical preparation. The indicia can indicate directly that the composition is free of the (R)-7,8-saturated-4,5-epoxy-morphinanium stereoisomer, or it can indicate the same indirectly, by stating for example that the composition is pure or 100% of the (S)-7,8-saturated-4,5-epoxy-morphinanium. The pharmaceutical composition can be for treating any of the conditions described herein. The pharmaceutical composition can contain an effective amount of the pure (S)-7,8-saturated-4,5-epoxy-morphinanium and can take any of the forms described below as if specifically recited in this summary, including, but not limited to, solutions, solids, semi-solids, enteric coated materials and the like.

These and other aspects of the invention are described in greater detail below.

### BRIEF DESCRIPTIONS OF DRAWINGS

FIG. 1a provides one of the potential structures of a 7,8-saturated-4,5-epoxy-morphinanium embodiment of the present invention. FIG. 1b illustrates in more detail the axial/equatorial relationships of substituents at nitrogen of (R) and (S) 7,8-saturated-4,5-epoxy-morphinanium embodiments of the present invention.

FIG. 2 illustrates a representative reaction scheme of the invention.

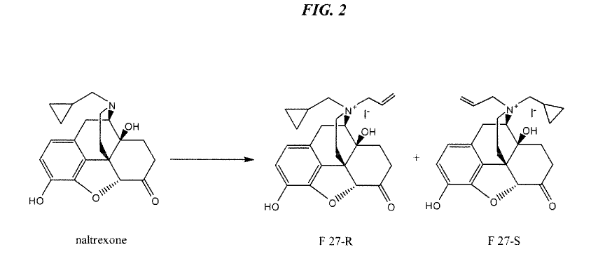

FIG. 3 provides a proton NMR spectrum of (S)-17-allyl-17-cyclopropylmethyl-4,5α-epoxy-3,14-saturatedxy-6-oxomorphinanium iodide.

FIG. 4 provides an NMR spectrum of (R)-17-allyl-17cyclopropylmethyl-4,5α-epoxy-3,14-saturatedxy-6-oxomorphinanium iodide.

### DETAILED DESCRIPTION OF THE INVENTION

The invention provides for (S)-7,8-saturated-4,5-epoxy-morphinanium compounds, synthetic routes for stereoselective synthesis of (S)-7,8-saturated-4,5-epoxy-morphinanium compounds, substantially pure (S)-7,8-saturated-4,5-epoxy-morphinanium compounds, crystals of substantially pure (S)-7,8-saturated-4,5-epoxy-morphinanium compounds, methods of analysis of (S)-7,8-saturated-4,5-epoxy-morphinanium compounds, pharmaceutical preparations containing substantially pure (S)-7,8-saturated-4,5-epoxy-morphinanium compounds, and methods for their use.

(S)-7,8-saturated-4,5-epoxy-morphinaniums of the present invention have the structure:

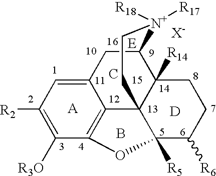

wherein X is a counterion and R₁₇ and R₁₈ are selected to result in an (S) configuration about the nitrogen in conformity with the Cahn, Ingold, Prelog configuration assignment rules, and R₁₈ and R₁₇ are C₁-C₈ alkyls or C₁-C₆ alkyls. R₃ may be a hydroxyl protecting group. The counterion can be any counterion, including a zwitterion. Preferably the counterion pharmaceutically acceptable. Counterions include halides, sulfates, phosphates, nitrates, and anionic-charged organic species. The halide can be iodide, bromide, chloride, fluoride, or combinations thereof. In one embodiment the halide is iodide. In an embodiment, the halide is bromide. The anionic-charged organic species may be a sulfonate or carboxylate.

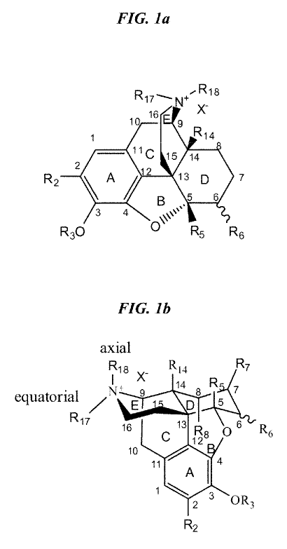

FIG. 1 provides one of the potential structures of a 7,8-saturated-4,5-epoxy-morphinanium embodiment of the present invention.

The term “acyl”, whether used alone, or within a term such as “acylamino”, denotes a radical provided by the residue after removal of hydroxyl from an organic acid. The term “acylamino” embraces an amine radical substituted with an acyl group. An examples of an “acylamino” radical is acetylamine (CH₃C(═O)—NH—). The term “aryloxy” denotes a radical provided by the residue after removal of hydrido from a hydroxy-substituted aryl moiety (e.g., phenol).

As used herein, “alkanoyl” refers to a-C (═O)-alkyl group, wherein alkyl is as previously defined. Exemplary alkanoyl groups include acetyl (ethanoyl), n-propanoyl, n-butanoyl, 2-methylpropanoyl, n-pentanoyl, 2-methylbutanoyl, 3-methylbutanoyl, 2,2-dimethylpropanoyl, heptanoyl, decanoyl, and palmitoyl.

The term “alkenyl” includes unsaturated aliphatic groups analogous in length and possible substitution to the alkyls described above, but that contain at least one double bond and must contain at least two carbon atoms. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term “lower alkylene” herein refers to those alkylene groups having from about 1 to about 6 carbon atoms. The term “alkenyl” includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Alkenylene”, in general, refers to an alkylene group containing at least one carbon-carbon double bond. Exemplary alkenylene groups include, for example, ethenylene (—CH═CH—) and propenylene (—CH═CHCH₂—). Preferred alkenylene groups have from 2 to about 4 carbons.

The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having two or more alkoxy radicals attached to the alkyl radical, that is, to form monoalkoxyalkyl and dialkoxyalkyl radicals. The “alkoxy” or “alkoxyalkyl” radicals may be further substituted with one or more halo atoms, such as fluoro chloro or bromo to provide “haloalkoxy” or “haloalkoxyalkyl” radicals. Examples of “alkoxy” radicals include methoxy butoxy and trifluoromethoxy.

“Alkyl” in general, refers to an aliphatic hydrocarbon group which may be straight, branched or cyclic having from 1 to about 10 carbon atoms in the chain, and all combinations and subcombinations of ranges therein, e.g., a cycloalkyl, branched cycloalkylalkyl, a branched alkylcycloalkyl having 4-10 carbon atoms. The term “alkyl” includes both “unsubstituted alkyls” and “substituted alkyls,” the latter of which refers to alkyl moieties having substituents replacing a hydrogen on one or more carbons of the backbone. “Lower alkyl” refers to an alkyl group having 1 to about 6 carbon atoms. Alkyl groups include, but are not limited to, methyl, ethyl, n-propyl, isopropyl, n-butyl, isobutyl, t-butyl, n-pentyl, cyclopentyl, isopentyl, neopentyl, n-hexyl, isohexyl, cyclohexyl, cyclooctyl, adamantyl, 3-methylpentyl, 2-dimethylbutyl, and 2,3-dimethylbutyl, cyclopropylmethyl and cyclobutylmethyl. Alkyl substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. The term “aralkyl” embraces aryl-substituted alkyl radicals such as benzyl, diphenylmethyl, triphenylmethyl, phenethyl, phenylpropyl, and diphenethyl. The terms benzyl and phenylmethyl are interchangeable. The term “n-alkyl” means a straight chain (i.e. unbranched) unsubstituted alkyl group. “Branched” refers to an alkyl group in which a lower alkyl group, such as methyl, ethyl or propyl, is attached to a linear alkyl chain.

An “alkylating agent” is a compound that can be reacted with a starting material to bind, typically covalently, an alkyl group to the starting material. The alkylating agent typically includes a leaving group that is separated from the alkyl group at the time of attachment to the starting material. Leaving groups may be, for example, halogens, halogenated sulfonates or halogenated acetates. An example of an alkylating agent is cyclopropylmethyl iodide.

The term “alkylsilyl” denotes a silyl radical substituted with an alkyl group. The term “alkylsilyloxy” denotes a silyloxy radical (—O—Si—) substituted with an alkyl group. An example of an “alkylsilyloxy” radical is —O—Si-t-BuMe).

The term “alkylsulfinyl” embraces radicals containing a linear or branched alkyl radical, of one to ten carbon atoms, attached to a divalent —S(═O)— atom. The term “arylsulfinyl” embraces aryl radicals attached to a divalent —S(═O)— atom (e.g., —S═OAr).

The term “alkylthio” embraces radicals containing a linear or branched alkyl radical, of one to ten carbon atoms, attached to a divalent sulfur atom. The term “arylsulfenyl” embraces aryl radicals attached to a divalent sulfur atom (—SAr) An example of “alkylthio” is methylthio, (CH₃—(S)—).

The term “alkynyl” includes unsaturated aliphatic groups analogous in length and possible substitution to the alkyls described above, but which contain at least one triple bond and two carbon atoms. For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups.

The term “amido” when used by itself or with other terms such as “amidoalkyl”, “N-monoalkylamido”, “N-monoarylamido”, “N,N-dialkylamido”, “N-alkyl-N-arylamido”, “N-alkyl-N-hydroxyamido” and “N-alkyl-N-hydroxyamidoalkyl”, embraces a carbonyl radical substituted with an amino radical. The terms “N-alkylamido” and “N,N-dialkylamido” denote amido groups which have been substituted with one alkyl radical and with two alkyl radicals, respectively. The terms “N-monoarylamido” and “N-alkyl-N-arylamido” denote amido radicals substituted, respectively, with one aryl radical, and one alkyl and one aryl radical. The term “N-alkyl-N-hydroxyamido” embraces amido radicals substituted with a hydroxyl radical and with an alkyl radical. The term “N-alkyl-N-hydroxyamidoalkyl” embraces alkyl radicals substituted with an N-alkyl-N-hydroxyamido radical. The term “amidoalkyl” embraces alkyl radicals substituted with amido radicals.

The term “aminoalkyl” embraces alkyl radicals substituted with amine radicals. The term “alkylaminoalkyl” embraces aminoalkyl radicals having the nitrogen atom substituted with an alkyl radical. The term “amidino” denotes an —C(═NH)—NH₂ radical. The term “cyanoamidino” denotes an —C(═N—CN)—NH₂ radical.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronapthyl, indane and biphenyl.

“Aryl-substituted alkyl”, in general, refers to an linear alkyl group, preferably a lower alkyl group, substituted at a carbon with an optionally substituted aryl group, preferably an optionally substituted phenyl ring. Exemplary aryl-substituted alkyl groups include, for example, phenylmethyl, phenylethyl and 3-(4-methylphenyl)propyl.

The term “carbocycle” is intended to mean any stable 3- to 7-membered monocyclic or bicyclic or 7- to 13-membered bicyclic or tricyclic, any of which may be saturated, partially unsaturated, or aromatic. Examples of such carbocycles include, but are not limited to, cyclopropyl, cyclobutyl, cyclopentyl, cyclohexyl, cycloheptyl, adamantyl, cyclooctyl, [3.3.0]bicyclooctane, [4.3.0]bicyclononane, [4.4.0]bicyclodecane (decalin), [2.2.2]bicyclooctane, fluorenyl, phenyl, naphthyl, indanyl, adamantyl, or tetrahydronaphthyl (tetralin). Preferred “carbocycle” are cyclopropyl, cyclobutyl, cyclopentyl, and cyclohexyl.

The term “cycloalkyl” embraces radicals having three to ten carbon atoms, such as cyclopropyl cyclobutyl, cyclopentyl, cyclohexyl and cycloheptyl.

“Cycloalkyl-substituted alkyl”, in general, refers to a linear alkyl group, preferably a lower alkyl group, substituted at a terminal carbon with a cycloalkyl group, preferably a C₃-C₈ cycloalkyl group. Typical cycloalkyl-substituted alkyl groups include cyclohexylmethyl, cyclohexylethyl, cyclopentylethyl, cyclopentylpropyl, cyclopropylmethyl and the like.

“Cycloalkenyl”, in general, refers to an olefinically unsaturated cycloalkyl group having from about 4 to about 10 carbons, and all combinations and subcombinations of ranges therein. In some embodiments, the cycloalkenyl group is a C₅-C₈ cycloalkenyl group, i.e., a cycloalkenyl group having from about 5 to about 8 carbons.

“Dipolar aprotic” solvents are protophilic solvents that cannot donate labile hydrogen atoms and that exhibit a permanent dipole moment. Examples include acetone, ethyl acetate, dimethyl sulfoxide (DMSO), dimethyl formamide (DMF) and N-methylpyrrolidone.

“Dipolar protic” solvents are those that can donate labile hydrogen atoms and that exhibit a permanent dipole moment. Examples include water, alcohols such as 2-propanol, ethanol, methanol, carboxylic acids such as formic acid, acetic acid, and propionic acid.

The phrase “does not substantially cross,” as used herein, means that less than about 20% by weight of the compound employed in the present methods crosses the bloodbrain barrier, preferably less than about 15% by weight, more preferably less than about 10% by weight, even more preferably less than about 5% by weight and most preferably 0% by weight of the compound crosses the blood-brain barrier.

The term “halo” means halogens such as fluorine, chlorine, bromine or iodine atoms. The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either a bromo, chloro or a fluoro atom within the radical. Dihalo radicals may have two or more of the same halo atoms or a combination of different halo radicals and polyhaloalkyl radicals may have more than two of the same halo atoms or a combination of different halo radicals.

As used herein, the term “heterocycle” or “heterocyclic ring” is intended to mean a stable 5- to 7-membered monocyclic or bicyclic or 7- to 14-membered bicyclic heterocyclic ring which is saturated, partially unsaturated, or unsaturated (aromatic), and which consists of carbon atoms and 1, 2, 3 or 4 heteroatoms independently selected from the group consisting of N, O and S and including any bicyclic group in which any of the above-defined heterocyclic rings is fused to a benzene ring. Examples of saturated heterocyclic radicals include pyrrolidyl and morpholinyl.

The term “hydroxyalkyl” embraces linear or branched alkyl radicals having one to about ten carbon atoms any one of which may be substituted with one or more hydroxyl radicals.

The term “hydrido” denotes a single hydrogen atom (H). This hydrido radical may be attached, for example, to an oxygen atom to form a hydroxyl radical or two hydrido radicals may be attached to a carbon atom to form a methylene (—CH₂—) radical.

The terms “N-alkylamino” and “N,N-dialkylamino” denote amine groups which have been substituted with one alkyl radical and with two alkyl radicals, respectively.

As used herein, “N-oxide” refers to compounds wherein the basic nitrogen atom of either a heteroaromatic ring or tertiary amine is oxidized to give a quaternary nitrogen bearing a positive formal charge and an attached oxygen atom bearing a negative formal charge.

“Organic solvent” has its common ordinary meaning to those of skill in this art. Exemplary organic solvents useful in the invention include, but are not limited to tetrahydrofuran, acetone, hexane, ether, chloroform, acetic acid, acetonitrile, chloroform, cyclohexane, methanol, and toluene. Anhydrous organic solvents are included.

As used herein, “patient” refers to animals, including mammals, preferably humans.

As used herein, “peripheral” or “peripherally-acting” refers to an agent that acts outside of the central nervous system. As used herein, “centrally-acting” refers to an agent that acts within the central nervous system (CNS). The term “peripheral” designates that the compound acts primarily on physiological systems and components external to the central nervous system. The phrase “substantially no CNS activity,” as used herein, means that less than about 20% of the pharmacological activity of the compounds employed in the present methods is exhibited in the CNS, preferably less than about 15%, more preferably less than about 10%, even more preferably less than about 5% and most preferably 0% of the pharmacological activity of the compounds employed in the present methods is exhibited in the CNS.

As used herein, “prodrug” refers to compounds specifically designed to maximize the amount of active species that reaches the desired site of reaction that are of themselves typically inactive or minimally active for the activity desired, but through biotransformation are converted into biologically active metabolites.

As used herein, “pharmaceutically acceptable” refers to those compounds, materials, compositions, and or dosage forms that are, within the scope of sound medical judgment, suitable for contact with the tissues of human beings and animals without excessive toxicity, irritation, allergic response, or other problem complications commensurate with a reasonable benefit/risk ratio. As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine. Certain acidic or basic compounds of the present invention may exist as zwitterions. All forms of the compounds, including free acid, free base and zwitterions, are contemplated to be within the scope of the present invention. It is well known in the art that compounds containing both amino and carboxyl groups often exist in equilibrium with their zwitterionic forms. Thus, any of the compounds described herein throughout that contain, for example, both amino and carboxyl groups, also include reference to their corresponding zwitterions.

As used herein, the term “side effect” refers to a consequence other than the one (s) for which an agent or measure is used, as the adverse effects produced by a drug, especially on a tissue or organ system other then the one sought to be benefited by its administration.

As used herein, “stereoisomers” refers to compounds that have identical chemical constitution, but differ as regards the arrangement of the atoms or groups in space.

The terms “sulfamyl” or “sulfonamidyl”, whether alone or used with terms such as “N-alkylsulfamyl”, “N-arylsulfamyl”, “N,N-dialkylsulfamyl” and “N-alkyl-N-arylsulfamyl”, denotes a sulfonyl radical substituted with an amine radical, forming a sulfonamide (—SO₂ NH₂). The terms “N-alkylsulfamyl” and “N,N-dialkylsulfamyl” denote sulfamyl radicals substituted, respectively, with one alkyl radical, a cycloalkyl ring, or two alkyl radicals. The terms “N-arylsulfamyl” and “N-alkyl-N-arylsulfamyl” denote sulfamyl radicals substituted, respectively, with one aryl radical, and one alkyl and one aryl radical.

The term “sulfonyl”, whether used alone or linked to other terms such as alkylsulfonyl, denotes respectively divalent radicals —SO₂—. “Alkylsulfonyl”, embraces alkyl radicals attached to a sulfonyl radical, where alkyl is defined as above. The term “arylsulfonyl” embraces sulfonyl radicals substituted with an aryl radical.

“Tertiary amines” has its common, ordinary meaning. In general, the tertiary amines useful in the invention have the general formula:

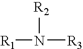

wherein R₁, R₂, and R₃ are identical or a combination of different straight or branched chain alkyl groups, alkenyl groups, alkylene groups, alkenylene groups, cycloalkyl groups, cycloalkyl-substituted alkyl groups, cycloalkenyl groups, alkoxy groups, alkoxy-alkyl groups, acyl groups, aryl groups, aryl-substituted alkyl groups, and heterocyclic groups. Exemplary tertiary amines useful according to the invention are those where R₁₋₃ is an alkyl group of the formula (CₙH₂ₙ₊₁, n=1-4), or aralkyl group of the formula (C₆H₅(CH₂)ₙ— [n=1-2]. Exemplary tertiary amines useful according to the invention also are cycloalkyl tertiary amines (e.g., N-methylmorpholine, N-methylpyrrolidine, N-methylpiperidine), pyridine and Proton Sponged (N,N,N′,N′-tetramethyl-1,8-naphthalene).

An (S)-7,8-saturated-4,5-epoxy-morphinanium exhibits properties different from those of its corresponding (R)-7,8-saturated-4,5-epoxy-morphinanium and different properties from a mixture of the (S) and (R) of the particular 7,8-saturated-4,5-epoxy-morphinanium. Those properties may include mobility on chromatography columns, biological and functional activity, and crystal structure. It is believed that the in vivo clearance rate, the side-effect profile, and the like may also differ from one (R)-7,8-saturated-4,5-epoxy-morphinanium or mixtures of the (R)-7,8-saturated-4,5-epoxy-morphinanium and (S)-7,8-saturated-4,5-epoxy-morphinanium. Pure (S)-7,8-saturated-4,5-epoxy-morphinaniums may behave as agonists of peripheral opioid receptors as, for example, inhibiting gastrointestinal transit. As a consequence, (S)-7,8-saturated-4,5-epoxy-morphinanium activity may be interfered with or antagonized by (R)-7,8-saturated-4,5-epoxy-morphinanium activity in mixtures containing both (R)-7,8-saturated-4,5-epoxy-morphinaniums and (S)-7,8-saturated-4,5-epoxy-morphinaniums. It therefore is highly desirable to have (S)-7,8-saturated-4,5-epoxy-morphinaniums in isolated and substantially pure form.

In one aspect of the invention, methods for the synthesis of (S)-7,8-saturated-4,5-epoxy-morphinanium are provided. An (S)-7,8-saturated-4,5-epoxy-morphinanium may be produced at a purity of greater than or equal to 10%, 20%, 30%, 40%, 50%, 60%, 70%, 75%, 80%, 85%, 90%, 95%, 97%, 98%, 98.5%, 99%, and 99.5% area under the curve (AUC) based on chromatographic techniques. In an embodiment, the purity of an (S)-7,8-saturated-4,5-epoxy-morphinanium is 98% or greater. The amount of a corresponding (R)-7,8-saturated-4,5-epoxy-morphinanium in the purified (S)-7,8-saturated-4,5-epoxy-morphinanium may be less than or equal to about 90%, 80%, 70%, 60%, 50%, 40%, 30%, 20%, 10%, 5%, 3%, 2%, 1%, 0.5%, 0.3%, 0.2%, 0.1% (AUC) or undetectable by chromatographic techniques described herein. It will be appreciated by the skilled artisan that the detection of the methods will depend upon the detection and quantitation limits of the employed technique. Quantitation Limit is the lowest amount of (R)-7,8-saturated-4,5-epoxy-morphinanium that can be consistently measured and reported, regardless of variations in laboratories, analysts, instruments or reagent lots. Detection Limit is the lowest amount of (R)-7,8-saturated-4,5-epoxy-morphinanium in a sample which can be detected but not necessarily quantitated as an exact value. In one embodiment of the invention the detection limit is 0.1% and the quantitation limit is 0.2%. In yet another embodiment the detection limit is 0.02% and the quantitation limit is 0.05%.

Synthesis of a number of 7,8-saturated-4,5-epoxy-morphinaniums of the present invention may be by the direct alkylation of tertiary morphinan, such as oxymorphone. The phenolic OH group of oxymorphone may be unprotected or protected. The (S)-7,8-saturated-4,5-epoxy-morphinanium salt may include a counterion such as iodide, that can then be exchanged for a more preferred counterion, for example, bromide. A useful starting material in the synthesis of number of (S)-7,8-saturated-4,5-epoxy-morphinaniums is disclosed herein as oxymorphone, which may be obtained at about 95% yield through the demethylation of oxycodone, for example, with boron tribromide. Alternatively, the oxymorphone may be obtained through commercial sources.

An alkylation reaction may be performed in a solvent, or solvent system, that may be anhydrous. The solvent system may be a single solvent or may include a combination of two or more solvents. Suitable solvent systems may include dipolar aprotic solvents such as N-methylpyrrolidone (NMP), dimethyl formamide (DMF), hexamethylphosphoramide (HMPA), acetone, 1,4-dioxane and acetonitrile, and dipolar protic solvents such as 2-propanol. Solvent systems may also include dipolar aprotic solvents in combination with aliphatic ethers, such as tetrahydrofuran (THF), 1,2-dimethoxyethane (glyme), diethyleneglycol dimethyl ether (diglyme), 1,4-dioxane, methyl t-butyl ether (methyl 1,1,-dimethylethyl ether, or 2-methyl-2-methoxypropane) diethyl ether, other polar solvents may also be included in some embodiments. For instance, the solvent system may include acetone, methylethylketone, diethylketone (3-pentanone), and t-butylmethylketone (3,3-dimethylbutan-2-one). Alkylation solvent systems may also include aliphatic or alicyclic congeners of any of the compounds disclosed above. Solvent systems may include two or more solvents in any proportion and appropriate proportions for a particular alkylation reaction may be determined through routine experimentation.

The solvent may be used at a ratio of less than, greater than, or equal to about 1, 2, 3, 4, 5, 10 or more volumes. In some cases it may be preferred to minimize the amount of solvent used, such as when product is to be transferred from the solvent using a liquid/liquid extraction or when product is to be crystallized or when the solvent is to be removed from the product.

The alkylating agent may be added to the starting material in various molar ratios, such as less than 8, 12, 16, 20, 24 or greater than 24 equivalents per equivalent of starting material. Reaction efficiency (production of 7,8-saturated-4,5-epoxy-morphinanium) may be substantially independent of the amount of alkylating agent used in some cases.

In one set of embodiments, alkylation may be performed using the Finkelstein reaction. For example, an alkyl halide, such as cyclopropylmethyl chloride, can be combined with a halide salt, such as sodium iodide, to continuously supply a reactive halogenated alkylating agent, such as cyclopropylmethyl iodide, that is replenished as it is consumed.

Starting materials may be alkylated at atmospheric pressure in an open vessel or under pressure. The reaction may be conducted such that the temperature is maintained or controlled over the reaction time at a prescribed temperature using methods/equipment as are known in the art. One device for maintaining a controlled temperature throughout the alkylation reaction is a heater/chiller unit. Controlling the temperature throughout the alkylation reaction inhibits or reduces temperature fluctuations. The reaction may need to proceed for a number of hours, for example, up to about 22 hours, or 15 to 22 hours, or 16 to 20 hours. Reaction times may in some cases be shortened through the use of microwave irradiation.

In some embodiments, the (S)-7,8-saturated-4,5-epoxy-morphinanium may be isolated from the solvent in which it is produced. For example, the solvent may be removed from a residue containing the (S)-7,8-saturated-4,5-epoxy-morphinanium, or any (S)-7,8-saturated-4,5-epoxy-morphinanium may be transferred from the alkylation solvent to a transfer solvent. Transfer solvents may be polar or non-polar and may have boiling points below 100° C. Transfer solvents may include esters, aldehydes, ethers, alcohols, aliphatic hydrocarbons, aromatic hydrocarbons and halogenated hydrocarbons. Specific transfer solvents include, for example, dioxane, ethyl acetate, isopropyl acetate, methanol, ethanol, dichloromethane, acetonitrile, water, aqueous HBr, heptane, and MTBE.

Any residue obtained from the solvent may be worked up to purify and isolate the (S) product. Purification and isolation may be done using methods known to those skilled in the art, such as by using separation techniques like chromatography, recrystallization, or combinations of various separation techniques as are known the art. In one embodiment, flash chromatography using a C18 column may be used. For example, a CombiFlash™ Sq 16x from ISCO using a Reverse Phase (C18) ReliSep column may be used. Analytic HPLC may be performed, for example, on a Phenomenex Prodigy 5 um OD53 100A column and purification performed on a semi-prep Phenomenex Prodigy 5 um OD53 100A column. Different solvents, such as aqueous methanol solvent modified with 0.2% HBr, may be employed with methanol content varying from, for example, about 2.5% to about 50%. The (S)-7,8-saturated-4,5-epoxy-morphinanium may be purified using recrystallization. The process may be repeated until desired purity of product is obtained. In one embodiment, the (S)-7,8-saturated-4,5-epoxy-morphinanium is recrystallized at least two times, three times, or four or more times to achieve the desired level of purity. For example, an (S)-7,8-saturated-4,5-epoxy-morphinanium may be obtained at purities of greater than or equal to 50%, 80%, 85%, 90%, 95%, 97%, 98%, 98.5%, 99.8% (AUC) based on chromatographic techniques. Any impurities may include the starting material, with no detectable (R)-7,8-saturated-4,5-epoxy-morphinanium. Recrystallization may be achieved using a single solvent, or a combination of solvents. In one embodiment, recrystallization is achieved by dissolving (S)-7,8-saturated-4,5-epoxy-morphinanium in a polar solvent, and then adding a less polar cosolvent. In another recrystallization embodiment, (S)-7,8-saturated-4,5-epoxy-morphinanium is purified by recrystallization from a solvent, for example, methanol, and a cosolvent, such as CH₂Cl₂/IPA (6:1). The recrystallization is repeated to achieve desired purity.

The (S)-7,8-saturated-4,5-epoxy-morphinanium, and its derivatives, may be produced in the salt form. Derivatives such as zwitterions of (S)-7,8-saturated-4,5-epoxy-morphinanium are included. The (S)-7,8-saturated-4,5-epoxy-morphinanium may include a positively charged quaternary ammonium group and may be paired with a anion such as a monovalent or multivalent anion. These anions may include, for example, halides, sulfates, phosphates, nitrates and charged organic species such as sulfonates and carboxylates. Preferred anions include halides such as bromide, chloride, iodide, fluoride, and combinations thereof. In some embodiments, bromide is most preferred. Specific anions may be chosen based on factors such as, for example, reactivity, solubility, stability, activity, cost, availability and toxicity.

Anions of the (S)-7,8-saturated-4,5-epoxy-morphinanium salt can be exchanged for alternative anions. When an alternative anion is desired, an aqueous solution of an (S)-7,8-saturated-4,5-epoxy-morphinanium salt can be passed over an anion exchange resin column to exchange some or all of the counterion of the (S)-7,8-saturated-4,5-epoxy-morphinanium salt for a preferred alternative counterion. Examples of anion exchange resins include AG 1-X8 in a 100 to 200 mesh grade, available from Bio-Rad. In another embodiment, the (S)-7,8-saturated-4,5-epoxy-morphinanium cation can be retained on a cation exchange resin and can then be exchanged by removing the (S)-7,8-saturated-4,5-epoxy-morphinanium from the resin with a salt solution that includes a preferred anion, such as bromide or chloride, forming the desired (S)-7,8-saturated-4,5-epoxy-morphinanium salt in solution.

The (S)-7,8-saturated-4,5-epoxy-morphinaniums of the present invention have numerous utilities. One aspect of the invention is an (S)-7,8-saturated-4,5-epoxy-morphinanium as a chromatographic standard in identifying and distinguishing its counterpart (R)-7,8-saturated-4,5-epoxy-morphinanium from other components in a sample in a chromatographic separation. Another aspect of the invention is the use of an (S)-7,8-saturated-4,5-epoxy-morphinanium as a chromatographic standard in identifying and distinguishing an (S)-7,8-saturated-4,5-epoxy-morphinanium in a mixture containing an (S)-7,8-saturated-4,5-epoxy-morphinanium and an (R)-7,8-saturated-4,5-epoxy-morphinanium counterpart. An isolated (S)-7,8-saturated-4,5-epoxy-morphinanium is also useful in the development of protocols for purifying and distinguishing an (S)-7,8-saturated-4,5-epoxy-morphinanium from an (R)-7,8-saturated-4,5-epoxy-morphinanium in reaction mixtures.

The (S)-7,8-saturated-4,5-epoxy-morphinaniums may be provided in a kit form with instruction for its use as a standard. The kit may further comprise an authentic (R)-7,8-saturated-4,5-epoxy-morphinanium as a standard. The (S)-7,8-saturated-4,5-epoxy-morphinanium for use as a standard preferably has a purity of 99.8% or greater with no detectable stereoisomeric (R)-7,8-saturated-4,5-epoxy-morphinanium.

One embodiment of the invention is a method of resolving and identifying an (S)-7,8-saturated-4,5-epoxy-morphinanium and a counterpart (R)-7,8-saturated-4,5-epoxy-morphinanium in a solution of 7,8-saturated-4,5-epoxy-morphinanium. The (S)-7,8-saturated-4,5-epoxy-morphinanium also is useful in HPLC assay methods of quantifying an amount of an (S)-7,8-saturated-4,5-epoxy-morphinanium in a composition or mixture in which the method comprises applying a sample of the composition or mixture to a chromatography column, resolving the components of the composition or mixture, and calculating the amount of an (S)-7,8-saturated-4,5-epoxy-morphinanium in the sample by comparing the percentage of a resolved component in the sample with the percentage of a standard concentration of an (S)-7,8-saturated-4,5-epoxy-morphinanium. The method is particularly useful in reverse phase HPLC chromatography. The (S)-7,8-saturated-4,5-epoxy-morphinanium of the present invention by virtue of its agonist activity on opioid receptors, is useful as a standard of agonist activity in in vitro and in vivo opioid receptor assays such as those described herein.

The (S)-7,8-saturated-4,5-epoxy-morphinaniums of the present invention can be used to regulate a condition mediated by one or more peripheral opioid receptors, prophylactically or therapeutically, to agonize peripheral opioid receptors, in particular peripheral mu opioid receptors. The subjects being administered an (S)-7,8-saturated-4,5-epoxy-morphinanium may receive treatment acutely, chronically or on an as needed basis.

The subjects to which the (S)-7,8-saturated-4,5-epoxy-morphinanium may be administered are vertebrates, in particular mammals. In one embodiment the mammal is a human, nonhuman primate, dog, cat, sheep, goat, horse, cow, pig and rodent. In one embodiment, the mammal is a human.

Mu and other opioid receptors exist in the gastrointestinal tract. Of the major classes of opioid receptors in the GI tract, mu receptors are principally involved in modulation of GI activity. Kappa opioid receptors may also play a role (Manara L et al Ann. Rev. Phamacol. Toxicol, 1985, 25:249-73). In general, the (S)-7,8-saturated-4,5-epoxy-morphinanium is used to prevent or treat conditions associated with the need for activation or modulation of opioid receptors, in particular, peripheral opioid receptors. Of interest is the use of (S)-7,8-saturated-4,5-epoxy-morphinaniums to prevent or treat conditions associated with the need for activation or modulation of opioid receptors in the GI tract, in particular mu opioid receptors. Such conditions which may be prevented or treated include diarrhea and used to prevent or inhibit certain forms of gastrointestinal dysfunction including certain forms of inflammatory bowel syndrome, and eating and digestive disorders.

In one embodiment, an (S)-7,8-saturated-4,5-epoxy-morphinanium of the present invention can be used to treat diarrhea. Gastrointestinal function is regulated, at least in part, by one or more opioid receptors as well as endogenous opioids. Opioid antagonists are known to increase gastrointestinal motility and may thus be used effectively as a treatment for constipation. Opioid agonists on the other hand, in particular peripheral opioid agonists such as loperamide are known to decrease gastrointestinal motility and can be useful in treating diarrhea in mammals. Agonist (S)-7,8-saturated-4,5-epoxy-morphinaniums of the present invention, as an opioid agonist, can be administered to a patient in need of treatment for diarrhea. Diarrhea as used herein is defined as one or more of the following: 1) stool loose in consistency; 2) passing of greater than 3 stools per day; and/or 3) passing a stool volume of >200 g (150 ml) per day. An (S)-7,8-saturated-4,5-epoxy-morphinanium is administered in an amount effective to prolong the transit time of intestinal contents resulting in reduced fecal volume, increase fecal viscosity and bulk density and diminished loss of fluid and electrolytes.

The (S)-7,8-saturated-4,5-epoxy-morphinaniums of the present invention by virtue of their opioid agonist activity is useful in the prevention and treatment of diarrhea having diverse etiology including acute and chronic forms of diarrhea, including chronic functional (idiopathic) diarrhea.

Acute diarrhea or short-term diarrhea as used herein is diarrhea lasting less than 1 week in duration, typically 1 to 3 days. Chronic diarrhea, ongoing or prolonged diarrhea as used herein is diarrhea lasting 1 week or longer duration. Chronic diarrhea may last for months or even years and may be continuous or intermittent. Various forms and causes of diarrhea which may benefit from treatment using an (S)-7,8-saturated-4,5-epoxy-morphinanium of the present invention include, but are not limited to those described below.

Viral gastroenteritis or “stomach flu” caused by any virus including but not limited to rotavirus, Norwalk virus, cytomegalovirus, herpes simples virus, Hepatitis virus, and Adenovirus, is amenable to treatment using an (S)-7,8-saturated-4,5-epoxy-morphinaniums of the present invention.

Food poisoning and traveler's diarrhea which occur from eating food or drinking water contaminated with organisms such as bacteria and parasites are amenable to treatment using an (S)-7,8-saturated-4,5-epoxy-morphinanium of the present invention. Bacteria commonly causing diarrhea include Escherichia coli, Salmonella, Shigella, Clostridia, Campylobacter, Yersinia, and Listeria. Parasites which cause diarrhea include Giardia lamblia, Entarnaeba histolvtica, and Cryptosporidium. Fungus which may cause diarrhea includes Candida.

Certain medical conditions can also lead to diarrhea including malabsorption syndromes such as lactose intolerance, celiac disease (sprue or gluten malabsorption), cystic fibrosis, intolerance to the protein in cows milk or other specific foods like beans, or fruits. Allergies to specific foods is another condition which may cause gastrointestinal irritation and/or allergic reaction leading to diarrhea. Typical food allergens include peanuts, corn and shellfish. Diarrhea caused by or associated with these medical conditions is amendable to treatment using an (S)-7,8-saturated-4,5-epoxy-morphinanium of the present invention.

Other medical conditions that lead to diarrhea, in particular chronic diarrhea include inflammatory bowel diseases which include Crohn's disease and ulcerative colitis, irritable bowel syndrome (IBS) and immune deficiency may also benefit from an (S)-7,8-saturated-4,5-epoxy-morphinanium of the present invention to prevent or treat the diarrhea.

An (S)-7,8-saturated-4,5-epoxy-morphinanium of the present invention may also be useful in preventing and treating diarrhea caused by medications and/or therapies such as antibiotics, laxatives containing magnesium, chemotherapeutics for cancer treatment and high dose radiation therapy.

Diarrhea is also associated with Zollinge®-Ellison syndrome, nerve disorders such as autonomic neuropathy or diabetic neuropathy, carcinoid syndrome, vasoactive intestinal polypeptide-secreting tumor, and anatomical conditions of the gastrointestinal tract including short bowel syndrome, gastrectomy, bowel resection with or without ileostomy or colostomy, and removal of the gall bladder. Such conditions may be amenable to treatment using an (S)-7,8-saturated-4,5-epoxy-morphinanium of the present invention.

An (S)-7,8-saturated-4,5-epoxy-morphinanium of the present invention may be administered through any route, oral or parenteral, including intraperitoneal, intravenous, vaginal, rectal, intramuscular, subcutaneously, aerosol, nasal spray, transmucosal, transdermal, topical, colonic, and the like for the prevention and treatment of diarrhea.

An (S)-7,8-saturated-4,5-epoxy-morphinanium of the present invention may also be useful in methods of reducing a volume of discharge from a ileostomy or colostomy in a subject. The (S)-7,8-saturated-4,5-epoxy-morphinanium may be provided in an amount effective to reduce the volume of discharge from the ostomy, compared to the volume of discharge from the ostomy in its absence. An (S)-7,8-saturated-4,5-epoxy-morphinanium may also be useful in controlling the rate of discharge from an ostomy, in particular in reducing the rate of discharge in a subject in need of lower rate of discharge.

According to another aspect of the invention, a method is provided for inhibiting gastrointestinal motility in a subject. The method involves administering to a subject in need of such inhibition a pharmaceutical composition containing an (S)-7,8-saturated-4,5-epoxy-morphinanium of the present invention in an amount effective to inhibit gastrointestinal motility in the subject. According to the invention, the (S)-7,8-saturated-4,5-epoxy-morphinanium may be administered in conjunction with another motility inhibiting agent that is not an (S)-7,8-saturated-4,5-epoxy-morphinanium. In one embodiment, the agent is an opioid or an opioid agonist. Opioids and opioid agonists are described above. In another embodiment, the agent is not an opioid or an opioid agonist. Examples of such nonopioid gastrointestinal motility inhibiting agents include, for example, cisapride, antacids, aluminum hydroxide, magnesium aluminum silicate, magnesium carbonate, magnesium hydroxide, calcium carbonate, polycarbophil, simethicone, hyoscyamine, atropine, furazolidone, difenoxin, octreotide, lansoprazole, kaolin, pectin, activated charcoal, sulphaguanidine, succinylsulphathiazole, phthalylsulphathiazole, bismuth-containing preparations such as bismuth aluminate, bismuth subcarbonate, bismuth subcitrate, bismuth citrate, tripotassium dicitrato bismuthate, bismuth tartrate, bismuth subsalicylate, bismuth subnitrate and bismuth subgallate, opium tincture (paregoric), herbal medicines and plant-derived anti-diarrheal agents. Further such agents include benzodiazepine compounds, antispasmodic, selective serotonin reuptake inhibitors (SSRIs), cholecystokinin (CCK) receptor antagonists, natural killer (NK) receptor antagonists, Corticotropin Releasing Factor (CRF) receptor agonists, antacids, GI relaxants, anti-gas compounds, pentosan polysulfate, anti-emetic dopamine D2 antagonists, gonadotrophin-releasing hormone analogues (leuprolide), corticotrophin-1 antagonists, neurokinin 2 receptor antagonists, cholecystokinin-1 antagonists, beta-blockers, anti-esophageal reflux agents, anti-inflammatory agents, 5HT₁ agonists, 5HT₃ antagonists, 5HT₄ antagonists, bile salt sequestering agents, bulk-forming agents, alpha₂-adrenergic agonists, antidepressants such as tricyclic antidepressants. Additional such agents include antimuscarinic agents, ganglion blocking agents, hormones and hormone analogs, and motilin receptor antagonists. Antimuscarinic agents include belladonna alkaloids, quaternary ammonium antimuscarinic compounds and tertiary amine antimuscarinic compounds. Examples of belladonna alkaloids include belladonna leaf extracts, belladonna tincture, and belladonna extract. Examples of quaternary ammonium antimuscarinic agents include Anisotropine or Anisotropine methylbromide (Valpin), Clidinium or Clidinium bromide (Quarzan), Glycopyrrolate (Robinul), Hexocyclium methylsulfate (Tral), Homatropine, Ipratropium or Ipratropium bromide, Isopropamide or Isopropamide iodide (Darbid), Mepenzolate or Mepenzolate bromide (Cantil), Methantheline or Methantheline bromide (Banthine), Methscopolamine or Methscopolamine bromide (Pamine), Oxyphenonium, and Propantheline or Propantheline bromide. Examples of tertiary amine antimuscarinic agents include Atropine, Dicyclomine or Dicyclomine hydrochloride (Bentyl and others), Flavoxate hydrochloride (Urispas), Oxybutynin or Oxybutynin chloride (Ditropan), Oxyphencyclimine or Oxyphencyclimine hydrochloride (Daricon), Propiverine, Scopolamine, Tolterodine, and Tridihexethyl or Tridihexethyl chloride (Pathilon). Other antimuscarinic agents include Pirenzepine, Telenzepine, AF-DX116, Methoctranine, Himbacine, and Hexahydrosiladifenidol. Ganglion blocking agents include synthetic amines such as Hexamethonium, Mecamylamine, Tetraethylammonium, and Acetylcholine. Examples of hormones or hormone analogs that are anti-gastrointestinal motility agents include: somatostatin and somatostatin receptor agonists. Examples of somatostatin analogs include octreotide (e.g., Sandostatin®) and vapreotide. Motilin antagonists include (Phe3, Leu-13) porcine motilin, 214^{th} American Chemical Society (ACS) Meeting (Part V); Highlights from Medicinal Chemistry Poster Session, Wednesday 10 September, Las Vegas, Nev., (1997), Iddb Meeting Report September 7-11 (1997); and ANQ-1 1 125, Peeters T. L., et al., Biochem. Biophys. Res. Commun., Vol. 198(2), pp. 411-416 (1994).

In another embodiment, an (S)-7,8-saturated-4,5-epoxy-morphinanium of the present invention may be used to treat eating and digestive disorders. Eating disorders and digestive disorders amenable to treatment using an (S)-7,8-saturated-4,5-epoxy-morphinanium according to the invention comprise, but are not limited to, the regulation of pathological imbalanced appetite, loss of appetite or diminished appetite, induced for example by pregnancy, cancer, infectious diseases such as influenza, HCV or HIV, as a result of catabolism, cachexy, anorexia, especially anorexia nervosa, dysorexia, dysponderosis, adiposity, bulimia, obesity, gastroparesis, especially neurogenic gastroparesis, diabetic gastroparesis, myogenic gastroparesis or gastroparesis induced by drugs, gastroatonia, gastroparalysis or enteroparesis, and stenosis of the gastrointestinal tract, especially stenosis of the pyloras.

Pain has been defined in a variety of ways. For example, pain can be defined as the perception by a subject of noxious stimuli that produces a withdrawal reaction by the subject Analgesia, is the reduction of pain perception. Agents that selectively block an animal's response to a strong stimulus without obtunding general behavior or motor function are referred to as analgesics. Opiates and opioid agonists affect pain via interaction with specific opioid receptors. An (S)-7,8-saturated-4,5-epoxy-morphinanium of the present invention, in having agonist activity, may find use in the treatment of pain.

The pain managed or treated can be associated with any of a wide variety of disorders, conditions, or diseases. “Pain” as used herein, unless specifically noted otherwise, is meant to encompass pain of any duration and frequency, including, but not limited to, acute pain, chronic pain, intermittent pain, and the like. Causes of pain may be identifiable or unidentifiable. Where identifiable, the origin of pain may be, for example, of malignant, non-malignant, infectious, non-infectious, or autoimmune origin. One embodiment is the management of pain associated with diseases, disorders, or conditions that require short-term therapy, e.g., dental procedures, bone fractures, outpatient surgeries, for which therapy involves treatment over a period of hours up to 3 days. Of particular interest is the management of pain associated with disorders, diseases, or conditions that require long-term therapy, e.g., chronic and/or persistent diseases or conditions for which therapy involves treatment over a period of several days (e.g., about 3 days to 10 days), to several weeks (e.g., about 2 weeks or 4 weeks to 6 weeks), to several months or years, up to and including the remaining lifetime of the subject. Subjects who are not presently suffering from a disease or condition, but who are susceptible to such may also benefit from prophylactic pain management using the compositions and methods of the invention, e.g., prior to traumatic surgery. Pain amenable to therapy according to the invention may involve prolonged episodes of pain alternating with pain-free intervals, or substantially unremitting pain that varies in severity.

In general, pain can be nociceptive, somatogenic, neurogenic, or psychogenic. Somatogenic pain can be muscular or skeletal (i.e., osteoarthritis, lumbosacral back pain, posttraumatic, myofascial), visceral (i.e., pancreatitis, ulcer, irritable bowel), ischemic (i.e., arteriosclerosis obliterans), or related to the progression of cancer (e.g., malignant or non-malignant). Neurogenic pain can be due to posttraumatic and postoperative neuralgia, can be related to neuropathies (i.e., diabetes, toxicity, etc.), and can be related to nerve entrapment, facial neuralgia, perineal neuralgia, postamputation, thalamic, causalgia, and reflex sympathetic dystrophy.

Specific examples of conditions, diseases, disorders, and origins of pain amenable to management according to the present invention include, but are not necessarily limited to, cancer pain (e.g., metastasis or non-metastatic cancer), inflammatory disease pain, neuropathic pain, postoperative pain, iatrogenic pain (e.g., pain following invasive procedures or high dose radiation therapy, e.g., involving scar tissue formation resulting in a debilitating compromise of freedom of motion and substantial pain), complex regional pain syndromes, failed-back pain (e.g., acute or chronic back pain), soft tissue pain, joints and bone pain, central pain, injury (e.g., debilitating injuries, e.g., paraplegia, quadriplegia, etc., as well as non-debilitating injury (e.g., to back, neck, spine, joints, legs, arms, hands, feet, etc.)), arthritic pain (e.g., rheumatoid arthritis, osteoarthritis, arthritic symptoms of unknown etiology, etc.), hereditary disease (e.g., sickle cell anemia), infectious disease and resulting syndromes (e.g., Lyme disease, AIDS, etc.), headaches (e.g., migraines), causalgia, hyperesthesia, sympathetic dystrophy, phantom limb syndrome, denervation, and the like. Pain can be associated with any portion(s) of the body, e.g., the musculoskeletal system, visceral organs, skin, nervous system, etc.

The methods of the invention can be used to manage pain in patients who are opioid naïve or who are no longer opioid naïve. Exemplary opioid naïve patients are those who have not received long-term opioid therapy for pain management. Exemplary non-opioid naïve patients are those who have received short-term or long-term opioid therapy and have developed tolerance, dependence, or other undesirable side effect. For example, patients who have intractable adverse side effects with oral, intravenous, or intrathecal morphine, transdermal fentanyl patches, or conventionally administered subcutaneous infusions of fentanyl, morphine or other opioid can achieve good analgesia and maintain favorable side-effects profiles with delivery of an (S)-7,8-saturated-4,5-epoxy-morphinanium and derivatives thereof.

The term “pain management or treatment” is used here to generally describe regression, suppression, or mitigation of pain so as to make the subject more comfortable as determined by subjective criteria, objective criteria, or both. In general, pain is assessed subjectively by patient report, with the health professional taking into consideration the patient's age, cultural background, environment, and other psychological background factors known to alter a person's subjective reaction to pain.

As mentioned above, the (S)-7,8-saturated-4,5-epoxy-morphinanium can be administered together with a therapeutic agent that is not an (S)-7,8-saturated-4,5-epoxy-morphinanium, including but not limited, therapeutic agents that arc pain relieving agents. In one embodiment, the pain relieving agent is an opioid or opioid agonist. In another embodiment, the pain relieving agent is a nonopioid pain relieving agent such as a corticosteroid or a nonsteroidal anti-inflammatory drug (NSAID) or Acetaminophen. Pain relieving agents include: Alfentanil Hydrochloride; Aminobenzoate Potassium; Aminobenzoate Sodium; Anidoxime; Anileridine; Anileridine Hydrochloride; Anilopam Hydrochloride; Anirolac; Antipyrine; Aspirin; Benoxaprofen; Benzydamine Hydrochloride; Bicifadine Hydrochloride; Brifentanil Hydrochloride; Bromadoline Maleate; Bromfenac Sodium; Buprenorphine Hydrochloride; Butacetin; Butixirate; Butorphanol; Butorphanol Tartrate; Carbamazepine; Carbaspirin Calcium; Carbiphene Hydrochloride; Carfentanil Citrate; Ciprefadol Succinate; Ciramadol; Ciramadol Hydrochloride; Clonixeril; Clonixin; Codeine; Codeine Phosphate; Codeine Sulfate; Conorphone Hydrochloride; Cyclazocine; Dexoxadrol Hydrochloride; Dexpemedolac; Dezocine; Diflunisal; Saturatedeodeine Bitartrate; Dimefadane; Dipyrone; Doxpicomine Hydrochloride; Drinidene; Enadoline Hydrochloride; Epirizole; Ergotamine Tartrate; Ethoxazene Hydrochloride; Etofenamate; Eugenol; Fenoprofen; Fenoprofen Calcium; Fentanyl Citrate; Floctafenine; Flufenisal; Flunixin; Flunixin Meglumine; Flupirtine Maleate; Fluproquazone; Fluradoline Hydrochloride; Flurbiprofen; Hydromorphone Hydrochloride; Ibufenac; Indoprofen; Ketazocine; Ketorfanol; Ketorolac Tromethamine; Letimide Hydrochloride; Levomethadyl Acetate; Levomethadyl Acetate Hydrochloride; Levonantradol Hydrochloride; Levorphanol Tartrate; Lofemizole Hydrochloride; Lofentanil Oxalate; Lorcinadol; Lornoxicam; Magnesium Salicylate; Mefenamic Acid; Menabitan Hydrochloride; Meperidine Hydrochloride; Meptazinol Hydrochloride; Methadone Hydrochloride; Methadyl Acetate; Methopholine; Methotrimeprazine; Metkephamid Acetate; Mimbane Hydrochloride; Mirfentanil Hydrochloride; Molinazone; Morphine Sulfate; Moxazocine; Nabitan Hydrochloride; Nalbuphine Hydrochloride; Nalmexone Hydrochloride; Namoxyrate; Nantradol Hydrochloride; Naproxen; Naproxen Sodium; Naproxol; Nefopam Hydrochloride; Nexeridine Hydrochloride; Noracymethadol Hydrochloride; Ocfentanil Hydrochloride; Octazamide; Olvanil; Oxetorone Fumarate; Oxycodone; Oxycodone Hydrochloride; Oxycodone Terephthalate; Oxymorphone Hydrochloride; Pemedolac; Pentamorphone; Pentazocine; Pentazocine Hydrochloride; Pentazocine Lactate; Phenazopyridine Hydrochloride; Phenyramidol Hydrochloride; Picenadol Hydrochloride; Pinadoline; Pirfenidone; Piroxicam Olamine; Pravadoline Maleate; Prodilidine Hydrochloride; Profadol Hydrochloride; Propiram Fumarate; Propoxyphene Hydrochloride; Propoxyphene Napsylate; Proxazole; Proxazole Citrate; Proxorphan Tartrate; Pyrroliphene Hydrochloride; Remifentanil Hydrochloride; Salcolex; Salethamide Maleate; Salicylamide; Salicylate Meglumine; Salsalate; Sodium Salicylate; Spiradoline Mesylate; Sufentanil; Sufentanil Citrate; Talmetacin; Talniflumate; Talosalate; Tazadolene Succinate; Tebufelone; Tetrydamine; Tifurac Sodium; Tilidine Hydrochloride; Tiopinac; Tonazocine Mesylate; Tramadol Hydrochloride; Trefentanil Hydrochloride; Trolamine; Veradoline Hydrochloride; Verilopam Hydrochloride; Volazocine; Xorphanol Mesylate; Xylazine Hydrochloride; Zenazocine Mesylate; Zomepirac Sodium; Zucapsaicin, and combinations thereof.

Hyperalgesia is an increased sensitivity to pain or enhanced intensity of pain sensation. Hyperalgesia can result when a subject is hypersensitive to a stimulus, resulting in an exaggerated pain response to a given stimulus. Hyperalgesia is often the result of a local inflammatory state and may follow trauma or injury to body tissue. Inflammation may follow, or be associated with, local infection, blisters, boils, skin injury such as cuts, scrapes, burns, sunburns, abrasions, surgical incisions, inflammatory skin conditions such as poison ivy, allergic rashes, insect bites and stings, and joint inflammation. An (S)-7,8-saturated-4,5-epoxy-morphinanium of the present invention can be used to prevent and treat peripheral hyperalgesia and to reduce pain and/or symptoms resulting from inflammation. As used herein, hyperalgesia includes pruritis, or itching, and the (S)-7,8-saturated-4,5-epoxy-morphinanium may be used as an anti-pruritic treatment.

The compositions and methods herein are intended for the preventions and treatment of hyperalgesia association with numerous inflammatory conditions and injuries. The compositions and methods provided herein may be used to treat a variety of hyperalgesic conditions associated with burns, including, but not limited to, thermal, radiation, chemical, sun and wind burns, abrasions, including, for example, corneal abrasions, bruises, contusions, frostbite, rashes, including, for example, allergic heat and contact dermatitis, such as, for example, poison ivy and diaper rashes, acne, insect bites/stings, skin ulcers, including, but not limited to, diabetic and decubitus ulcers, mucositis, inflammation, for example, periodontal inflammation, orthodontic inflammation, inflammation/irritation arising from use of a cosmetic or skin care product, inflammatory conjunctivitis, hemorrhoids and venereal inflammations, gingivitis, bronchitis, laryngitis, sore throat, singles, fungal irritation, for example, athlete's foot and jock itch, fever blisters, boils, plantar's warts or vaginal lesions, including, for example, mycotic and sexually transmitted vaginal lesions.

Hyperalgesic conditions associated with skin surfaces include burns, including but not limited to, thermal, radiation, chemical, sun and wind burns, abrasions such as, for example, corneal abrasions, bruises, contusions, frostbite, rashes including allergic, heat contact dermatitis (for example, poison ivy) and diaper rashes), acne insect bites/stings and skin ulcers (including diabetic and decubitus ulcers). Hyperalgesic conditions of the mouth, larynx and bronchium include mucositis, post-tooth extraction, periodontal inflammation, gingivitis, orthodontic inflammation, bronchitis, laryngitis and sore throat. Hyperalgesic conditions of the eyes include corneal abrasions, post-radial keratectomy and inflammatory conjunctivitis. Hyperalgesic conditions of the rectum/anus include hemorrhoids and venereal inflammations. Hyperalgesic conditions associated with infectious agents include shingles, fungal irritations (including athlete's foot and jock itch), fever blisters, boils, plantar's warts and vaginal lesions (including lesions associated with mycosis and sexually transmitted diseases). Hyperalgesic conditions may also be associated with recovery following surgery, such as recovery following lumpectomy, episiotomy, laparoscopy, arthroscopy, radial keratectomy and tooth extraction.

As a preventative or treatment for peripheral hyperalgesia, an (S)-7,8-saturated-4,5-epoxy-morphinanium can be administered using any pathway that provides for delivery of the compound to an afflicted area. Administration may be oral or parenteral. Methods of administration also include topical and local administration. (S)-7,8-saturated-4,5-epoxy-morphinaniums of the present invention may be applied to any body surface including skin, joints, eyes lips and mucosal membranes.

The stereoisomer (S)-7,8-saturated-4,5-epoxy-morphinanium may be delivered in combination with other compounds, such as those disclosed herein, that provide anti-hyperalgesic effects, including, but not limited to, pain medications, itching medications, anti-inflammatory agents, and the like. It may be administered with other compounds used to treat the conditions causing the inflammation, such as antivirals, antibacterials, antifungals, and anti-infectives. These other compounds may act and be administered locally or systemically and may be part of the same composition or may be administered separately. Such compounds are described in greater detail below.

Inflammation is often associated with an increase in Tumor Necrosis Factor (TNF) production and it is believed that a decrease in TNF production will result in a reduction in inflammation. Peripherally acting opioid agonists have been shown to decrease TNF production (U.S. Pat. No. 6,190,691). The peripherally selective k-opioid, asimadoline, has been shown to be a potent anti-arthritic agent in an adjuvant-induced arthritis animal model (Binder, W. and Walker, J. S. Br. J. Pharma 124:647-654). Thus the peripheral opioid agonist activity of the (S)-7,8-saturated-4,5-epoxy-morphinanium and derivatives thereof provide for prevention and treatment of inflammatory conditions. While not being bound by theory, the anti-inflammatory effect of an (S)-7,8-saturated-4,5-epoxy-morphinanium and derivatives thereof may be through inhibition of TNF production, directly or indirectly. The (S)-7,8-saturated-4,5-epoxy-morphinanium or derivatives thereof may be administered systemically or locally. An (S)-7,8-saturated-4,5-epoxy-morphinanium may be administered in combination with another TNF inhibitor such as loperamide and diphenoxylate or with other anti-inflammatory agents described herein.

Another aspect of the present invention is prevention and/or treatment of a systemic inflammatory condition, preferably inflammatory bowel disease, rheumatoid arthritis, cachexia, asthma, Crohn's disease, endotoxin shock, adult respiratory distress syndrome, ischemic/reperfusion damage, graft-versu(S)-host reactions, bone resorption, transplantation or lupus using an (S)-7,8-saturated-4,5-epoxy-morphinanium of the present invention or derivatives thereof.

In still another group of embodiments, the inflammatory condition amenable to treatment using an (S)-7,8-saturated-4,5-epoxy-morphinanium of the present invention or derivatives thereof is associated with multiple sclerosis, diabetes or wasting associated with acquired immunodeficiency syndrome (AIDS) or cancer.

In one group of embodiments, a skin inflammatory condition, preferably psoriasis, atopic dermatitis, UV-induced inflammation, contact dermatitis or inflammation induced by other drugs, including but not limited to RETIN-A (all-tran(S)-retinoic acid) is amenable to treatment using an (S)-7,8-saturated-4,5-epoxy-morphinanium of the present invention or derivative thereof.

Another aspect of the invention is a method of treating a non-allergic inflammatory skin condition comprising the administration of an (S)-7,8-saturated-4,5-epoxy-morphinanium of the present invention in an amount effective to treat the inflammatory condition. Non-allergic inflammatory skin conditions are associated with irritant contact dermatitis, psoriasis, eczema, pruritus, seborrheic dermatitis, nummular dermatitis, lichen planus, acne vulgaris, comedones, polymorphs, nodulokystic acne, conglobata, senile acne, secondary acne, medicinal acne, a keratinization disorder, and blistery dermatoses.

Certain patients who may be particularly amenable to treatment are patients having the symptoms of any one of the foregoing conditions. The patients may have failed to obtain relief or ceased to obtain relief or a consistent degree of relief of their symptoms using other therapies. Such patients are said to be refractory to the conventional treatments. The condition may be induced or a consequence of one or more diverse conditions including, but not limited to, a disease condition, a physical condition, a drug-induced condition, a physiological imbalance, stress, anxiety, and the like. The conditions may be an acute condition or chronic condition.

Subjects can be treated with a combination of the (S)-7,8-saturated-4,5-epoxy-morphinanium and a therapeutic agent other than the (S)-7,8-saturated-4,5-epoxy-morphinanium. In these circumstances the (S)-7,8-saturated-4,5-epoxy-morphinanium and the other therapeutic agent(s) are administered close enough in time such that the subject experiences the effects of the various agents as desired, which typically is at the same time. In some embodiments, the (S)-7,8-saturated-4,5-epoxy-morphinanium will be delivered first in time, in some embodiments second in time, and still in some embodiments at the same time. As discussed in greater detail below, the invention contemplates pharmaceutical preparations where the (S)-7,8-saturated-4,5-epoxy-morphinanium is administered in a formulation including another pharmaceutical agent. Included are solid, semisolid, liquid, controlled release and other such formulations.

One important class of therapeutic agent which can be part of the prevention and treatment protocol together with an (S)-7,8-saturated-4,5-epoxy-morphinanium are opioids. Use of an (S)-7,8-saturated-4,5-epoxy-morphinanium of the present invention, in combination with the opioid, may result in an enhanced and apparently synergistic inhibition of gastrointestinal transit. Thus, the present invention provides pharmaceutical compositions comprising an (S)-7,8-saturated-4,5-epoxy-morphinanium in combination with one or more opioids. This will permit alteration of doses. For example, where a lower dose of opioid is desirable in treating certain peripherally mediated conditions, such may be reached by combination with an (S)-7,8-saturated-4,5-epoxy-morphinanium treatment.

The opioid can be any pharmaceutically acceptable opioid. Common opioids are those selected from the group consisting of alfentanil, anileridine, asimadoline, bremazocine, burprenorphine, butorphanol, codeine, dezocine, diacetylmorphine (heroin), saturatedcodeine, diphenoxylate, fedotozine, fentanyl, funaltrexamine, hydrocodone, hydromorphone, levallorphan, levomethadyl acetate, levorphanol, loperamide, meperidine (pethidine), methadone, morphine, morphine-6-glucoronide, nalbuphine, nalorphine, opium, oxycodone, oxymorphone, pentazocine, propiram, propoxyphene, remifentanyl, sufentanil, tilidine, trimebutine, and tramadol.

Depending on the desired effect to be achieved the opioid may be administered parenterally or other systemic route to affect both the central nervous system (CNS) and peripheral opioid receptors. The desired effect of the opioid in combination with an (S)-7,8-saturated-4,5-epoxy-morphinanium of the present invention may be prevention or treatment of diarrhea, prevention or treatment of pain from any cause or etiology including prevention or treatment of peripheral hyperalgesia. When the indication is prevention or treatment of peripheral hyperalgesia, it is desirable to provide an opioid which does not have concomitant CNS effects or alternatively to administer the opioid topically or locally such that the opioid does not substantially cross the blood brain barrier but provide an effect on peripheral opioid receptors.

Opioids particularly useful for prevention or treatment of diarrhea or prevention or treatment of peripheral hyperalgesia in combination with an (S)-7,8-saturated-4,5-epoxy-morphinanium of the present invention include but are not limited to:
(i) loperamide [4-(p-chlorophenyl)-4-hydroxy-N-N-dimethyl-α,α-diphenyl-1-piperidinebutyramide hydrochloride]], loperamide analogs and related compounds as defined herein [see, U.S. Pat. Nos. 3,884,916 and 3,714,159; see, also U.S. Pat. Nos. 4,194,045, 4,116,963, 4,072,686, 4,069,223, 4,066,654.], N-oxides of loperamide and analogs, metabolites and prodrugs thereof and related compounds as defined herein [see, also, U.S. Pat. No. 4,824,853], and related compounds, such as (a), (b) and (c) as follows:
(a) 4-(aroylamino)pyridine-butanamide derivatives and N-oxides thereof as defined herein [see, also U.S. Pat. No. 4,990,521];
(b) 5-(1,1-diphenyl-3-(5- or 6-hydroxy-2-azabicyclo-(2.2.2)oct-2-yl)propyl)-2-alkyl-1,3,4-oxadiazoles, 5-(1,1-diphenyl-4-(cyclic amino)but-2-tran(S)-en-1-yl)-2-alkyl-1,3,4-oxadiazoles, 2-[5-(cyclic amino)-ethyl-10,11-saturated-5H-dibenzo[a,d]-cyclohepten-5-yl]-5-alkyl-1,3,4-oxadiazoles] and related compounds [see, U.S. Pat. Nos. 4,013,668, 3,996,214 and 4,012,393];
(c) 2-substituted-1-azabicyclo[2,2,2]octanes [see, U.S. Pat. No. 4,125,531];
(ii) 3-hydroxy-7-oxomorphinans and 3-hydroxy-7-oxoisomorphinans [see, e.g., U.S. Pat. No. 4,277,605]
(iii) amidinoureas as provided herein [see, also U.S. Pat. Nos. 4,326,075, 4,326,074, 4,203,920, 4,060,635, 4,115,564, 4,025,652] and 2-[(aminophenyl and amidophenyl)amino]-1-azacycloalkanes [see, U.S. Pat. No. 4,533,739];
(iv) metkephamid [H-L-Ty(R)-D-Ala-Bly-L-Phe-N(Me)Met-NH₂; see, e.g., U.S. Pat. No. 4,430,327; Burkhart et al. (1982) Peptides 3-869-871; Frederickson et al. (1991) Science 211:603-605] and other synthetic opioid peptides, such as H-Ty(R)-D-Nva-Phe-Orn-NH₂, H-Ty(R)-D-Nle-Phe-Orn-NH₂, H-Ty(R)-D-Arg-Phe-A₂bu-NH₂, H-Ty(R)-D-Arg-Phe-Ly(S)-NH₂, and H-Ly(S)-Ty(R)-D-Arg-Phe-Ly(S)-NH₂ [see, U.S. Pat. No. 5,312,899; see, also Gesellchen et al. (1981) Pept.: Synth., Struct., Funct., Proc. Am. Pept. Symp., 7^{th}; Rich et al., (Eds), Pierce Chem. Co., Rochford, Ill., pp. 621-62] that do not cross the blood brain barrier;
(v) propanamines as defined in U.S. Pat. No. 5,236,947 and the like.

An (S)-7,8-saturated-4,5-epoxy-morphinanium of the present invention may also be used to treat diarrhea in combination with other anti-diarrheal compounds and compositions. For example, an (S)-7,8-saturated-4,5-epoxy-morphinanium may be administered to a subject in combination with a known anti-diarrheal agent. Two or more compounds may be administered in a cocktail or the compounds may be administered separately using the same or different administration routes. Known anti-diarrheal agents include, for example, loperamide, loperamide analogs, N-oxides of loperamide and analogs, metabolites and prodrugs thereof, diphenoxylate, cisapride, antacids, aluminum hydroxide, magnesium aluminum silicate, magnesium carbonate, magnesium hydroxide, calcium carbonate, polycarbophil, simethicone, hyoscyamine, atropine, furazolidone, difenoxin, octreotide, lansoprazole, kaolin, pectin, activated charcoal, sulphaguanidine, succinylsulphathiazole, phthalylsulphathiazole, bismuth aluminate, bismuth subcarbonate, bismuth subcitrate, bismuth citrate, tripotassium dicitrato bismuthate, bismuth tartrate, bismuth subsalicylate, bismuth subnitrate and bismuth subgallate, opium tincture (paregoric), herbal medicines and plant-derived anti-diarrheal agents.

Other therapeutic agents which can be part of treatment protocols together with an (S)-7,8-saturated-4,5-epoxy-morphinanium of the present invention are irritable bowel syndrome (IBS) agents, antibiotics, antivirals, anti-fungals, anti-infectives, anti-inflammatory agents including anti-histamines, vasoconstrictors, anti-diarrheals, and the like.

IBS therapeutic agents which may be used in combination with an (S)-7,8-saturated-4,5-epoxy-morphinanium include, but are not limited to, benzodiazepine compounds, antispasmodic, selective serotonin reuptake inhibitors (SSRIs), cholecystokinin (CCK) receptor antagonists, motilin receptor agonists or antagonists, natural killer (NK) receptor antagonists, Corticotropin Releasing Factor (CRF) receptor agonists or antagonists, somatostatin receptor agonists, antacids, GI relaxants, anti-gas compounds, bismuth-containing preparations, pentosan polysulfate, anti-emetic dopamine D2 antagonists, prostaglandin E analogs, gonadotrophin-releasing hormone analogues (leuprolide), corticotrophin-1 antagonists, neurokinin 2 receptor antagonists, cholecystokinin-1 antagonists, beta-blockers, anti-esophageal reflux agents, anti-muscarinics, antidiarrheals, anti-inflammatory agents, anti-motility agents, 5HT₁ agonists, 5HT₃ antagonists, 5HT₄ antagonists, 5HT₄ agonists, bile salt sequestering agents, bulk-forming agents, alpha₂-adrenergic agonists, mineral oils, antidepressants, herbal medicines.

Specific examples of IBS therapeutic agents include, but are not limited to, the following:

Benzodiazepine compounds and analogs which act to suppress seizures through an interaction with gamma-aminobutyric acid (GABA) receptors of the A-type (GABA_{A}), for example, DIASTAT® and VALIUM®; LIBRIUM®; and ZANAX®.

SSRIs, for example, fluvoxamine; fluoxetine; paroxetine; sertraline; citalopram; venlafaxine; cericlamine; duloxetine; milnacipran; nefazodone; and cyanodothiepin (See The Year Drugs News, 1995 Edition, pp. 47-48 by Prous J. R.) and WO 97/29739.

CCK receptor antagonists, for example, devazepide; lorglumide; dexioxiglumide; loxiglumide, D'Amato, M. et al., Br. J. Pharmacol. Vol. 102(2), pp. 391-395 (1991); C1 988; L364,718; L3637260; L740,093 and LY288,513; CCK receptor antagonists disclosed in U.S. Pat. No. 5,220,017 Bruley-De(S)-Varannes, S, et al. Gastroenterol. Clin. Biol. Vol. 15.(10)₉ pp. 744-757 (1991), and Worker C: EUPHAR'99—Second European Congress of Pharmacology (Part IV) Budapest, Hungary Iddb Meeting Report 1999 Jul. 3-7.

Motilin receptor agonists or antagonists which include e.g. motilin agonist ABT-269, (erythromycin, 8,9-didehydro-N-dimethyl deoxo-4″,6,12-trideoxy-6,9-epoxy-N-ethyl), de(Nmethyl-N-ethyl-8,9-anhydroerythromycin A) and de(N-methyl)-N-isoprop-8,9anhydroerythromycin A), Sunazika T. et al. Chem. Pharm. Bull., Vol. 37(10), pp. 2687-2700 (1989); A-173508 (Abbot Laboratories); motilin antagonists (Phe3, Leu-13) porcine motilin, 214^{th} American Chemical Society (ACS) Meeting (Part V); Highlights from Medicinal Chemistry Poster Session, Wednesday 10 September, Las Vegas, Nev., (1997), Iddb Meeting Report September 7-11 (1997); and ANQ-1 1 125, Peeters T. L., et al., Biochem. Biophys. Res. Commun., Vol. 198(2), pp. 411-416 (1994).

NK receptor antagonists which include e.g. FK 888(Fujisawa); GR 205171 (Glaxo Wellcome); LY 303870 (Lilly); MK 869 (Merck); GR82334 (Glaxo Wellcome); L758298 (Merck); L 733060 (Merck); L 741671 (Merck); L 742694 (Merck); PD 154075 (Parke-Davis); S1 8523 (Servier); S19752 (Servier); OT 7100 (Otsuka); WIN 51708 (Sterling Winthrop); NKP-608A; TKA457; DNK333; CP-96345; CP-99994; CP122721; L-733060; L-741671; L742694; L-758298; L-754030; G(R)-203040; G(R)-205171; RP-67580; RP(R)-100893 (dapitant); RP(R)-107880; RP(R)-111905; FK-888; SDZ-NKT-343; MEN-10930; MEN-11149; (S)-18523; (S)-19752; PD-154075 (CAM-4261); S(R)-140333; LY-303870 (lanepitant); EP-00652218; EP00585913; L-737488; CGP-49823; WIN-51708; S(R)-48968 (saredutant); S(R)-144190; YM383336; ZD-7944; MEN-10627; G(R)-159897; RP(R)-106145; PD-147714 (CAM-2291); ZM253270; FK-224; MDL-1 05212A; MDL-105172A; L-743986; L-743986 analogs; (S)-16474; S(R)-1 42801 (osanetant); PD-161182; SB-223412; and SB-222200.

CRF receptor agonists or antagonists, e.g. as disclosed in WO 99/40089, AXC 2219, Antalarmin, NGD 1, CRA 0165, CRA 1000, CRA 1001.

Somatostatin receptor agonists, e.g. octreotide, vapreotide, lanreotide.

Anti-inflammatory compounds, particularly those of the immuno-modulatory type, for example, NSAIDS; Tumor Necrosis Factor (TNF, TNFa) inhibitors; basiliximab (e.g. SIMULECT®); daclizumab (e.g. ZENAPAX®); infliximab (e.g. REMICADE®); etanercept (e.g. ENBREL®)mycophenolate mofetil (e.g. CELLCEPT®) azathioprine (e.g. IMURAN®); tacrolimus (e.g. PROGRAF®); steroids; methotrexate and GI anti-inflammatory agents, for example, sulfasalazine (e.g. AZULFIDINE®); olsalazine (e.g. DIPENTUM®); and mesalatnine (e.g. ASACOL®, PENTASA®, ROWASA®).

Antacids, such as aluminum and magnesium antacids; and calcium hydroxides such as MAALOX®.

Anti-gas compounds, for example, simethicone marketed under the trade names MYLANTA® and MYLICON®; and enzyme preps including PHAZYME® and BEANO®.

Bismuth-containing preparations, for example, bismuth subsalicylate also known as PEPTO-BISMOL®.

Pentosan polysulfate, a heparin-like macromolecular carbohydrate derivative which chemically and structurally resembles glycosaminoglycans, marketed under the trade name ELMIRON®.

Anti-emetic dopamine D2 antagonists which include e.g. domperidone.

Prostaglandin E analogs, gonadotrophin-releasing hormone analogues (leuprolide), corticotrophin-1 antagonists, neurokinin 2 receptor antagonists, cholecystokinin-1 antagonists, beta-blockers.

Anti-esophageal reflux agents include but are not limited to PRILOSEC®.

Antispasmodics and anti-muscarinics include, but are not limited to, dicyclomine, oxybutyin (e.g., oxybutynin chloride), tolterodine (e.g., tolterodine tartarate), alverine anisotropine, atropine (e.g., atropine sulfate), belladonna, homatropine, homatropine methobromide, hyoscyamine (e.g., hyoscyamine sulfate), methscopolamine, scopolamine (e.g., scopolamine hydrochloride), clidinium, cimetropium, hexocyclium, pinaverium, otilonium, glycopyrrolate, and mebeverine.

Antidiarrheals include, but are not limited to, ipratropium, isoproparide, mepenzolate, propantheline, oxyphencylcimine, pirenzepine, diphenoxylate (e.g., diphenoxylate hydrochloride), atropine sulfate, alosetron hydrochloride, difenoxin hydrochloride, bismuth subsalicylate, lactobacillus acidophilus, trimebutine, asimadoline, and octreotide acetate.

Anti-inflammatory agents also include, but are not limited to, mesalamine, sulfsalazine, balsalazide disodium, hydrocortisone, and olsalazine sodium.

5HT₁ agonists include, but are not limited to, buspirone.

5HT₃ antagonists include, but are not limited to, ondansetron, cilansetron, and alosetron.

5HT₄ antagonists include, but are not limited to, piposcrod.

5HT₄ agonists include, but are not limited to, tegaserod (e.g., tegaserod maleate), and povcalopride.

Antidepressants include, but are not limited to, desiprimine, amitryptiline, imiprimine, fluoxetine, and paroxetine.

Other IBS therapeutic agents include dexloxiglumide, TAK-637, talnetant, SB 223412, AU 244, neurotrophin-3, GT 160-246, immunoglobulin (IgG), ramoplanin, risaxmin, rimethicone, darifenacine, zamifenacin, loxiglumide, misoprostil, leuprolide, domperidone, somatostatin analogues, phenyloin, NBI-34041, saredutant, and dexloxiglumide.

Antibiotics include, but are not limited to, tetracycline antibiotics, such as chlortetracycline, oxytetracycline, tetracycline, demethylchlortetracycline, metacycline, doxycycline, minocycline and rolitetracycline; such as kanamycin, amikacin, gentamicin C₁ₐ, C₂, C_{2b} or C₁, sisomicin, netilmicin, spectinomycin, streptomycin, tobramycin, neomycin B, dibekacin and kanendomycin; macrolides, such as maridomycin and erythromycin; lincomycins, such as clindamycine and lincomycin; penicillanic acid (6-APA)- and cephalosporanic acid (7-ACA)-derivatives having (6β- or 7β-acylamino groups, respectively, which are present in fermentatively, semi-synthetically or totally synthetically obtainable 6β-acylaminopenicillanic acid or 7β-acylaminocephalosporanic acid derivatives and/or 7β-acylaminocephalosporanic acid derivatives that are modified in the 3-position, such as penicillanic acid derivatives that have become known under the names penicillin G or V, such as phenethicillin, propicillin, nafcillin, oxycillin, cloxacillin, dicloxacillin, flucloxacillin, cyclacillin, epicillin, mecillinam, methicillin, azlocillin, sulbenicillin, ticarcillin, mezlocillin, piperacillin, carindacillin, azidocillin or ciclacillin, and cephalosporin derivatives that have become known under the names cefaclor, cefuroxime, cefazlur, cephacetrile, cefazolin, cephalexin, cefadroxil, cephaloglycin, cefoxitin, cephaloridine, cefsulodin, cefotiam, ceftazidine, cefonicid, cefotaxime, cefmenoxime, ceftizoxime, cephalothin, cephradine, cefamandol, cephanone, cephapirin, cefroxadin, cetatrizine, cefazedone, ceftrixon and ceforanid; and other β-lactam antibiotics of the clavam, penem and carbapenen type, such as moxalactam, clavulanic acid, nocardicine A, sulbactam, aztreonam and thienamycin; and other antibiotics including bicozamycin, novobiocin, chloramphenicol or thiamphenicol, rifampicin, fosfomycin, colistin, and vancomycin.

Antiviral agents include, but are not limited to, nucleoside analogs, nonnucleoside reverse transcriptase inhibitors, nucleoside reverse transcriptase inhibitors, protease inhibitors, integrase inhibitors, including the following: acemannan; acyclovir; acyclovir sodium; adefovir; alovudine; alvircept sudotox; amantadine hydrochloride; aranotin; anildone; atevirdine mesylate; pyridine; cidofovir; cipamfylline; cytarabine hydrochloride; delavirdine mesylate; desciclovir; didanosine; disoxaril; edoxudine; enviradene; enviroxime; famciclovir; famotine hydrochloride; fiacitabine; fialuridine; fosarilate; foscarnet sodium; fosfonet sodium; ganciclovir; ganciclovir sodium; idoxuridine; indinavir; kethoxal; lamivudine; lobucavir; lopinovir; memotine hydrochloride; methisazone; nelfinavir; nevirapine; penciclovir; pirodavir; ribavirin; rimantadine hydrochloride; ritonavir; saquinavir mesylate; somantadine hydrochloride; sorivudine; statolon; stavudine; tenofovir; tilorone hydrochloride; trifluridine; valacyclovir hydrochloride; vidarabine; vidarabine phosphate; vidarabine sodium phosphate; viroxime; zalcitabine; zerit; zidovudine (AZT); and zinviroxime.

Anti-infective agents include, but are not limited to, difloxacin hydrochloride; lauryl isoquinolinium bromide; moxalactam disodium; omidazole; pentisomicin; sarafloxacin hydrochloride; protease inhibitors of HIV and other retroviruses; integrase Inhibitors of HIV and other retroviruses; cefaclor (ceclor); acyclovir (zovirax); norfloxacin (noroxin); cefoxitin (mefoxin); cef roxime axetil (ceftin); ciprofloxacin (cipro); aminacrine hydrochloride; benzethonium chloride:bithionolate sodium; bromchlorenone; carbamide peroxide; cetalkonium chloride; cetylpyridinium chloride:chlorhexidine hydrochloride; clioquinol; domiphen bromide; fenticlor; fludazonium chloride; fuchsin, basic; furazolidone; gentian violet; halquinols; hexachlorophene:hydrogen peroxide; ichthammol; imidecyl iodine; iodine; isopropyl alcohol; mafenide acetate; meralein sodium; mercufenol chloride; mercury, ammoniated; methylbenzethonium chloride; nitrofurazone; nitromersol; octenidine hydrochloride; oxychlorosene; oxychlorosene sodium; parachlorophenol, camphorated; potassium permanganate; povidone-iodine; sepazonium chloride; silver nitrate; sulfadiazine, silver; symclosene; thimerfonate sodium; thimerosal: troclosene potassium.

Antifungal (antibiotics) include: polyenes such as Amphotericin-B, candicidin, dermostatin, filipin, fungichromin, hachimycin, hamycin, lucensomycin, mepartricin, natamycin, nystatin, pecilocin, perimycin; and others, such as azaserine, griseofulvin, oligomycins, pyrrolnitrin, siccanin, tubercidin and viridin. Antifungal synthetics include: allylamines such as naftifine and terbinafine; imidazoles such as bifonazole, butoconazole, chlordantoin, chlormidazole, cloconazole, clotrimazole, econazole, enilconazole, fenticonazole, isoconazole, ketoconazole, miconazole, omoconazole, oxiconazole nitrate, sulconazole and tioconazole; triazoles such as fluconazole, itraconazole, terconazole. Others include acrisorcin, amorolfine, biphenamine, bromosalicylchloranilide, buclosamide, chlophenesin, ciclopirox, cloxyquin, coparaffinate, diamthazole, saturatedchloride, exalamide, flucytosine, halethazole, hexetidine, loflucarban, nifuratel, potassium iodide, propionates, propionic acid, pyrithione, salicylanilide, sulbentine, tenonitrozole, tolciclate, tolindate, tolnaftate, tricetin, ujothion, and undecylenic acid. Antifungals also include the echinocandin class or antifungals, including caspofungin, micafungin, anidulafungin, aminocandin, and the like.

Vasoconstrictors include, but are not limited to, epinephrine, norepinephrine, pseudoephedrine, phenylephrine, oxymetazoline, propylhexedrine, naphazoline, tetrahydrolozine, xylometazonline, ethylnorepinephrine, methoxamine, phenylhexedrine, mephentermine, metaraminol, dopamine, dipivefrin, norphedrine and ciraxzoline may be advantageously used in the compositions and methods herein. Use of such should aid in reducing systemic delivery of the active antihyperalgesic agent.

The pharmaceutical preparations of the invention, when used alone or in cocktails, are administered in therapeutically effective amounts. A therapeutically effective amount will be determined by the parameters discussed below; but, in any event, is that amount which establishes a level of the drug(s) effective for treating a subject, such as a human subject, having one of the conditions described herein. An effective amount means that amount alone or with multiple doses, or the rate of delivery necessary to delay the onset of, lessen the severity of, or inhibit completely, lessen the progression of, or halt altogether the onset or progression of the condition being treated or a symptom associated therewith. In the case of diarrhea, an effective amount can be, for example, that amount which results in one or more of the following: 1) decreasing the frequency of bowel movements; 2) increasing the consistency of the stool, and/or 3) decreasing the stool volume to less than 200 g per day. In one embodiment, an effective amount is an amount that results in 3 or less per bowel movements per day, preferably 2 or less per day, more preferably 1 bowel movement per day. In certain instances, the amount is sufficient to decrease bowel movements within 12 hours of administration of the (S)-7,8-saturated-4,5-epoxy-morphinanium, 10 hours, 8 hours, 6 hours, 4 hours, 2 hours, 1 hour and even immediately upon administration, depending upon the mode of administration. Intravenous administration can produce an immediate effect. In restoring gastrointestinal function, an effective amount can be, for example, that amount necessary to increase oral-cecal transit time. For management or treatment of pain, an effective amount can be, for example, that amount to sufficient to make a subject more comfortable as determined by subjective criteria, objective criteria or both. In the case of peripheral hyperalgesia, an effective amount can be, for example, that amount which relieves a symptom of peripheral hyperalgesia such as hypersensitivity to pain or pruritis. For the prevention or treatment of inflammation, an effective amount can be, for example, the amount sufficient to reduce or lessen the redness, swelling, or tissue damage associated with the inflammation or to increase the mobility of an affected area such as a joint. When administered to a subject, effective amounts will depend, of course, on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation.

Oral doses of an (S)-7,8-saturated-4,5-epoxy-morphinanium of the present invention may be from about 0.05 to about 40 mg/kg, from 0.05 to about 20.0 mg/kg, from about 0.05 to about 10 mg/kg, or from about 0.05 to about 5 mg kg body weight per day. Parenteral administration, including intravenous and subcutaneous administration, may be from about 0.001 to 1.0 mg/kg, from about 0.01 to 1.0 mg/kg, or from about 0.1 to 1.0 mg/kg body weight depending on whether administration is as a bolus or is spread out over time such as with an I.V. drip. Doses ranging from about 0.05 to 0.5 mg/kg body weight may yield the desired results. Dosage may be adjusted appropriately to achieve desired drug levels, local or systemic, depending on the mode of administration. For example, it is expected that the dosage for oral administration of an (S)-7,8-saturated-4,5-epoxy-morphinanium in an enterically-coated formulation would be lower than in an immediate release oral formulation. In the event that the response in a patient is insufficient at such doses, even higher doses (or effectively higher dosage by a different, more localized delivery route) may be employed to the extent that the patient tolerance permits. Multiple doses per day are contemplated to achieve appropriate systemic levels of compounds. Appropriate systemic levels can be determined by, for example, measurement of the patient's peak or sustained plasma level of the drug. “Dose” and “dosage” are used interchangeably herein.

A variety of administration routes are available. The particular mode selected will depend, of course, upon the particular combination of drugs selected, the severity of the condition being treated, or prevented, the condition of the patient, and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, topical, transdermal, sublingual, intravenous infusion, pulmonary, intra-arterial, intra-adipose tissue, intra-lymphatic, intramuscular, intracavity, aerosol, aural (e.g., via eardrops), intranasal, inhalation, intra-articular, needleless injection, subcutaneous or intradermal (e.g., transdermal) delivery. For continuous infusion, a patient-controlled analgesia (PCA) device or an implantable drug delivery device may be employed. Oral, rectal, or topical administration may be important for prophylactic or long-term treatment. Preferred rectal modes of delivery include administration as a suppository or enema wash.

The pharmaceutical preparations may conveniently be presented in unit dosage form and may be prepared by any of the methods well known in the art of pharmacy. All methods include the step of bringing the compounds of the invention into association with a carrier which constitutes one or more accessory ingredients. In general, the compositions are prepared by uniformly and intimately bringing the compounds of the invention into association with a liquid carrier, a finely divided solid carrier, or both, and then, if necessary, shaping the product.

When administered, the pharmaceutical preparations of the invention are applied in pharmaceutically acceptable compositions. Such preparations may routinely contain salts, buffering agents, preservatives, compatible carriers, lubricants, and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but not limited to, those prepared from the following acids: hydrochloric, hydrobromic, sulfuric, nitric, phosphoric, maleic, acetic, salicylic, p-toluenesulfonic, tartaric, citric, methanesulfonic, formic, succinic, naphthalene-2-sulfonic, pamoic, 3-hydroxy-2-naphthalenecarboxylic, and benzene sulfonic.

It should be understood that when referring to a 7,8-saturated-4,5-epoxy-morphinanium, an (R)- and (S)-7,8-saturated-4,5-epoxy-morphinanium, and therapeutic agent(s) of the invention, it is meant to encompass salts of the same. Such salts are of a variety well known to those or ordinary skill in the art. When used in pharmaceutical preparations, the salts preferably are pharmaceutically-acceptable for use in humans. Bromide is an example of one such salt.

The pharmaceutical preparations of the present invention may include or be diluted into a pharmaceutically-acceptable carrier. The term “pharmaceutically-acceptable carrier” as used herein means one or more compatible solid or liquid fillers, diluents or encapsulating substances which are suitable for administration to a human or other mammal such as non-human primate, a dog, cat, horse, cow, sheep, pig, or goat. The term “carrier” denotes an organic or inorganic ingredient, natural or synthetic, with which the active ingredient is combined to facilitate the application. The carriers are capable of being commingled with the preparations of the present invention, and with each other, in a manner such that there is no interaction which would substantially impair the desired pharmaceutical efficacy or stability. Carrier formulations suitable for oral administration, for suppositories, and for parenteral administration, etc., can be found in Remington's Pharmaceutical Sciences, Mack Publishing Company, Easton, Pa.

Formulations may include a chelating agent, a buffering agent, an anti-oxidant and, optionally, an isotonicity agent, preferably pH adjusting or a permeation enhancer.

Chelating agents include, for example, ethylenediaminetetraacetic acid (EDTA) and derivatives thereof, citric acid and derivatives thereof, niacinamide and derivatives thereof, sodium desoxycholate and derivatives thereof, and L-glutamic acid, N, N-diacetic acid and derivatives thereof. EDTA derivatives include dipotassium edentate, disodium edentate, calcium disodium edentate, sodium edentate, trisodium edentate, and potassium edentate.

Buffering agents include those selected from the group consisting of citric acid, sodium citrate, sodium acetate, acetic acid, sodium phosphate and phosphoric acid, sodium ascorbate, tartaric acid, maleic acid, glycine, sodium lactate, lactic acid, ascorbic acid, imidazole, sodium bicarbonate and carbonic acid, sodium succinate and succinic acid, histidine, and sodium benzoate and benzoic acid, or combinations thereof.

Antioxidants include those selected from the group consisting of an ascorbic acid derivative, butylated hydroxy anisole, butylated hydroxy toluene, alkyl gallate, sodium meta-bisulfite, sodium bisulfite, sodium dithionite, sodium thioglycollate acid, sodium formaldehyde sulfoxylate, tocopheral and derivatives thereof, monothioglycerol, and sodium sulfite. The preferred antioxidant is monothioglycerol.

Isotonicity agents include those selected from the group consisting of sodium chloride, mannitol, lactose, dextrose, glycerol, and sorbitol.

Preservatives that can be used with the present compositions include benzyl alcohol, parabens, thimerosal, chlorobutanol and preferably benzalkonium chloride. Typically, the preservative will be present in a composition in a concentration of up to about 2% by weight. The exact concentration of the preservative, however, will vary depending upon the intended use and can be easily ascertained by one skilled in the art.

The compounds of the invention can be prepared in lyophilized compositions, preferably in the presence of a cryoprotecting agent such as mannitol, or lactose, sucrose, polyethylene glycol, and polyvinyl pyrrolidines. Cryoprotecting agents which result in a reconstitution pH of 6.0 or less are preferred. The invention therefore provides a lyophilized preparation of therapeutic agent(s) of the invention. The preparation can contain a cryoprotecting agent, such as mannitol or lactose, which is preferably neutral or acidic in water.

Oral, parenteral and suppository formulations of agents are well known and commercially available. The therapeutic agent(s) of the invention can be added to such well known formulations. It can be mixed together in solution or semi-solid solution in such formulations, can be provided in a suspension within such formulations or could be contained in particles within such formulations.

A product containing therapeutic agent(s) of the invention and, optionally, one or more other active agents can be configured as an oral dosage. The oral dosage may be a liquid, a semisolid or a solid. An opioid may optionally be included in the oral dosage. The oral dosage may be configured to release the therapeutic agent(s) of the invention before, after or simultaneously with the other agent (and/or the opioid). The oral dosage may be configured to have the therapeutic agent(s) of the invention and the other agents release completely in the stomach, release partially in the stomach and partially in the intestine, in the intestine, in the colon, partially in the stomach, or wholly in the colon. The oral dosage also may be configured whereby the release of the therapeutic agent(s) of the invention is confined to the stomach or intestine while the release of the other active agent is not so confined or is confined differently from the therapeutic agent(s) of the invention. For example, the therapeutic agent(s) of the invention may be an enterically coated core or pellets contained within a pill or capsule that releases the other agent first and releases the therapeutic agent(s) of the invention only after the therapeutic agent(s) of the invention passes through the stomach and into the intestine. The therapeutic agent(s) of the invention also can be in a sustained release material, whereby the therapeutic agent(s) of the invention is released throughout the gastrointestinal tract and the other agent is released on the same or a different schedule. The same objective for therapeutic agent(s) of the invention release can be achieved with immediate release of therapeutic agent(s) of the invention combined with enteric coated therapeutic agent(s) of the invention. In these instances, the other agent could be released immediately in the stomach, throughout the gastrointestinal tract or only in the intestine.

The materials useful for achieving these different release profiles are well known to those of ordinary skill in the art. Immediate release is obtainable by conventional tablets with binders which dissolve in the stomach. Coatings which dissolve at the pH of the stomach or which dissolve at elevated temperatures will achieve the same purpose. Release only in the intestine is achieved using conventional enteric coatings such as pH sensitive coatings which dissolve in the pH environment of the intestine (but not the stomach) or coatings which dissolve over time. Release throughout the gastrointestinal tract is achieved by using sustained-release materials and/or combinations of the immediate release systems and sustained and/or delayed intentional release systems (e.g., pellets which dissolve at different pHs).

In the event that it is desirable to release the therapeutic agent(s) of the invention first, the therapeutic agent(s) of the invention could be coated on the surface of the controlled release formulation in any pharmaceutically acceptable carrier suitable for such coatings and for permitting the release of the therapeutic agent(s) of the invention, such as in a temperature sensitive pharmaceutically acceptable carrier used for controlled release routinely. Other coatings which dissolve when placed in the body are well known to those of ordinary skill in the art.

The therapeutic agent(s) of the invention also may be mixed throughout a controlled release formulation, whereby it is released before, after or simultaneously with another agent. The therapeutic agent(s) of the invention may be free, that is, solubilized within the material of the formulation. The therapeutic agent(s) of the invention also may be in the form of vesicles, such as wax coated micropellets dispersed throughout the material of the formulation. The coated pellets can be fashioned to immediately release the therapeutic agent(s) of the invention based on temperature, pH or the like. The pellets also can be configured so as to delay the release of the therapeutic agent(s) of the invention, allowing the other agent a period of time to act before the therapeutic agent(s) of the invention exerts its effects. The therapeutic agent(s) of the invention pellets also can be configured to release the therapeutic agent(s) of the invention in virtually any sustained release pattern, including patterns exhibiting first order release kinetics or sigmoidal order release kinetics using materials of the prior art and well known to those of ordinary skill in the art.

The therapeutic agent(s) of the invention also can be contained within a core within the controlled release formulation. The core may have any one or any combination of the properties described above in connection with the pellets. The therapeutic agent(s) of the invention may be, for example, in a core coated with a material, dispersed throughout a material, coated onto a material or adsorbed into or throughout a material.

It should be understood that the pellets or core may be of virtually any type. They may be drug coated with a release material, drug interspersed throughout material, drug adsorbed into a material, and so on. The material may be erodible or nonerodible.

The therapeutic agent(s) of the invention, may be provided in particles. Particles as used herein means nano or microparticles (or in some instances larger) which can consist in whole or in part of the therapeutic agent(s) of the inventions or the other agents as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the antagonist in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as nonimmediate release formulations, with nonimmediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.” These formulations may be for any mode of administration.

Delivery systems specific for the gastrointestinal tract are roughly divided into three types: the first is a delayed release system designed to release a drug in response to, for example, a change in pH; the second is a timed-release system designed to release a drug after a predetermined time; and the third is a microflora enzyme system making use of the abundant enterobacteria in the lower part of the gastrointestinal tract (e.g., in a colonic site-directed release formulation).

An example of a delayed release system is one that uses, for example, an acrylic or cellulosic coating material and dissolves on pH change. Because of ease of preparation, many reports on such “enteric coatings” have been made. In general, an enteric coating is one which passes through the stomach without releasing substantial amounts of drug in the stomach (i.e., less than 10% release, 5% release and even 1% release in the stomach) and sufficiently disintegrating in the intestinal tract (by contact with approximately neutral or alkaline intestine juices) to allow the transport (active or passive) of the active agent through the walls of the intestinal tract.

Various in vitro tests for determining whether or not a coating is classified as an enteric coating have been published in the pharmacopoeia of various countries. A coating which remains intact for at least 2 hours, in contact with artificial gastric juices such as HCl of pH 1 at 36 to 38° C. and thereafter disintegrates within 30 minutes in artificial intestinal juices such as a KH₂PO₄ buffered solution of pH 6.8 is one example. One such well known system is EUDRAGIT material, commercially available and reported on by Behringer, Manchester University, Saale Co., and the like. Enteric coatings are discussed further, below.

A timed release system is represented by Time Erosion System (TES) by Fujisawa Pharmaceutical Co., Ltd. and Pulsincap by R. P. Scherer. According to these systems, the site of drug release is decided by the time of transit of a preparation in the gastrointestinal tract. Since the transit of a preparation in the gastrointestinal tract is largely influenced by the gastric emptying time, some time release systems are also enterically coated.

Systems making use of the enterobacteria can be classified into those utilizing degradation of azoaromatic polymers by an azo reductase produced from enterobacteria as reported by the group of Ohio University (M. Saffran, et al., Science, Vol. 233: 1081 (1986)) and the group of Utah University (J. Kopecek, et al., Pharmaceutical Research, 9(12), 1540-1545 (1992)); and those utilizing degradation of polysaccharides by beta-galactosidase of enterobacteria as reported by the group of Hebrew University (unexamined published Japanese patent application No. 5-50863 based on a PCT application) and the group of Freiberg University (K. H. Bauer et al., Pharmaceutical Research, 10(10), S218 (1993)). In addition, the system using chitosan degradable by chitosanase by Teikoku Seiyaku K. K. (unexamined published Japanese patent application No. 4-217924 and unexamined published Japanese patent application No. 4-225922) is also included.

The enteric coating is typically, although not necessarily, a polymeric material. Preferred enteric coating materials comprise bioerodible, gradually hydrolyzable and/or gradually water-soluble polymers. The “coating weight,” or relative amount of coating material per capsule, generally dictates the time interval between ingestion and drug release. Any coating should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention. The selection of the specific enteric coating material will depend on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; ease of application as a coating (substrate friendly); and economical practicality.

Suitable enteric coating materials include, but are not limited to: cellulosic polymers such as cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonium methylacrylate, ethyl acrylate, methyl methacrylate and/or ethyl methacrylate (e.g., those copolymers sold under the trade name EUDRAGIT); vinyl polymers and copolymers such as polyvinyl acetate, polyvinylacetate phthalate, vinylacetate crotonic acid copolymer, and ethylene-vinyl acetate copolymers; and shellac (purified lac). Combinations of different coating materials may also be used. Well known enteric coating material for use herein are those acrylic acid polymers and copolymers available under the trade name EUDRAGIT from Rohm Pharma (Germany). The EUDRAGIT series E, L, S, RL, RS and NE copolymers are available as solubilized in organic solvent, as an aqueous dispersion, or as a dry powder. The EUDRAGIT series RL, NE, and RS copolymers are insoluble in the gastrointestinal tract but are permeable and are used primarily for extended release. The EUDRAGIT series E copolymers dissolve in the stomach. The EUDRAGIT series L, L-30D and (S) copolymers are insoluble in stomach and dissolve in the intestine, and are thus most preferred herein.

A particular methacrylic copolymer is EUDRAGIT L, particularly L-30D and EUDRAGIT L 100-55. In EUDRAGIT L-30D, the ratio of free carboxyl groups to ester groups is approximately 1:1. Further, the copolymer is known to be insoluble in gastrointestinal fluids having pH below 5.5, generally 1.5-5.5, i.e., the pH generally present in the fluid of the upper gastrointestinal tract, but readily soluble or partially soluble at pH above 5.5, i.e., the pH generally present in the fluid of lower gastrointestinal tract. Another particular methacrylic acid polymer is EUDRAGIT S, which differs from EUDRAGIT L-30D in that the ratio of free carboxyl groups to ester groups is approximately 1:2. EUDRAGIT (S) is insoluble at pH below 5.5, but unlike EUDRAGIT L-30D, is poorly soluble in gastrointestinal fluids having a pH in the range of 5.5 to 7.0, such as in the small intestine. This copolymer is soluble at pH 7.0 and above, i.e., the pH generally found in the colon. EUDRAGIT (S) can be used alone as a coating to provide drug delivery in the large intestine. Alternatively, EUDRAGIT S, being poorly soluble in intestinal fluids below pH 7, can be used in combination with EUDRAGIT L-30D, soluble in intestinal fluids above pH 5.5, in order to provide a delayed release composition which can be formulated to deliver the active agent to various segments of the intestinal tract. The more EUDRAGIT L-30D used, the more proximal release and delivery begins, and the more EUDRAGIT (S) used, the more distal release and delivery begins. It will be appreciated by those skilled in the art that both EUDRAGIT L-30D and EUDRAGIT (S) can be replaced with other pharmaceutically acceptable polymers having similar pH solubility characteristics. In certain embodiments of the invention, the preferred enteric coating is ACRYL-EZE™ (methacrylic acid co-polymer type C; Colorcon, West Point, Pa.).

The enteric coating provides for controlled release of the active agent, such that drug release can be accomplished at some generally predictable location. The enteric coating also prevents exposure of the therapeutic agent and carrier to the epithelial and mucosal tissue of the buccal cavity, pharynx, esophagus, and stomach, and to the enzymes associated with these tissues. The enteric coating therefore helps to protect the active agent, carrier and a patient's internal tissue from any adverse event prior to drug release at the desired site of delivery. Furthermore, the coated material of the present invention allows optimization of drug absorption, active agent protection, and safety. Multiple enteric coatings targeted to release the active agent at various regions in the gastrointestinal tract would enable even more effective and sustained improved delivery throughout the gastrointestinal tract.

The coating can, and usually does, contain a plasticizer to prevent the formation of pores and cracks that would permit the penetration of the gastric fluids. Suitable plasticizers include, but are not limited to, triethyl citrate (Citroflex 2), triacetin (glyceiyl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating comprised of an anionic carboxylic acrylic polymer will usually contain approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g., hydroxypropylcellulose, acids and bases) to solubilize or disperse the coating material, and to improve coating performance and the coated product.

The coating can be applied to particles of the therapeutic agent(s), tablets of the therapeutic agent(s), capsules containing the therapeutic agent(s) and the like, using conventional coating methods and equipment. For example, an enteric coating can be applied to a capsule using a coating pan, an airless spray technique, fluidized bed coating equipment, or the like. Detailed information concerning materials, equipment and processes for preparing coated dosage forms may be found in Pharmaceutical Dosage Forms: Tablets, eds. Lieberman et al. (New York: Marcel Dekker, Inc., 1989), and in Ansel et al., Pharmaceutical Dosage Forms and Drug Delivery Systems, 6th Ed. (Media, Pa.: Williams & Wilkins, 1995). The coating thickness, as noted above, must be sufficient to ensure that the oral dosage form remains intact until the desired site of topical delivery in the lower intestinal tract is reached.

In another embodiment, drug dosage forms are provided that comprise an enterically coated, osmotically activated device housing a formulation of the invention. In this embodiment, the drug-containing formulation is encapsulated in a semipermeable membrane or barrier containing a small orifice. As known in the art with respect to so-called “osmotic pump” drug delivery devices, the semipermeable membrane allows passage of water in either direction, but not drug. Therefore, when the device is exposed to aqueous fluids, water will flow into the device due to the osmotic pressure differential between the interior and exterior of the device. As water flows into the device, the drug-containing formulation in the interior will be “pumped” out through the orifice. The rate of drug release will be equivalent to the inflow rate of water times the drug concentration. The rate of water influx and drug efflux can be controlled by the composition and size of the orifice of the device. Suitable materials for the semipermeable membrane include, but are not limited to, polyvinyl alcohol, polyvinyl chloride, semipermeable polyethylene glycols, semipermeable polyurethanes, semipermeable polyamides, semipermeable sulfonated polystyrenes and polystyrene derivatives; semipermeable poly(sodium styrenesulfonate), semipermeable poly(vinylbenzyltrimethylammonium chloride), and cellulosic polymers such as cellulose acetate, cellulose diacetate, cellulose triacetate, cellulose propionate, cellulose acetate propionate, cellulose acetate butyrate, cellulose trivalerate, cellulose trilmate, cellulose tripalmitate, cellulose trioctanoate, cellulose tripropionate, cellulose disuccinate, cellulose dipalmitate, cellulose dicylate, cellulose acetate succinate, cellulose propionate succinate, cellulose acetate octanoate, cellulose valerate palmitate, cellulose acetate heptanate, cellulose acetaldehyde dimethyl acetal, cellulose acetate ethylcarbamate, cellulose acetate methylcarbamate, cellulose dimethylaminoacetate and ethylcellulose.

In another embodiment, drug dosage forms are provided that comprise a sustained release coated device housing a formulation of the invention. In this embodiment, the drug-containing formulation is encapsulated in a sustained release membrane or film. The membrane may be semipermeable, as described above, A semipermeable membrane allows for the passage of water inside the coated device to dissolve the drug. The dissolved drug solution diffuses out through the semipermeable membrane. The rate of drug release depends upon the thickness of the coated film and the release of drug can begin in any part of the GI tract. Suitable membrane materials for such a membrane include ethylcellulose.

In another embodiment, drug dosage forms are provided that comprise a sustained release device housing a formulation of the invention. In this embodiment, the drug-containing formulation is uniformly mixed with a sustained release polymer. These sustained release polymers are high molecular weight water-soluble polymers, which when in contact with water, swell and create channels for water to diffuse inside and dissolve the drug. As the polymers swell and dissolve in water, more of drug is exposed to water for dissolution. Such a system is generally referred to as sustained release matrix. Suitable materials for such a device include hydropropyl methylcellulose, hydroxypropyl cellulose, hydroxyethyl cellulose and methyl cellulose.

In another embodiment, drug dosage forms are provided that comprise an enteric coated device housing a sustained release formulation of the invention. In this embodiment, the drug containing product described above is coated with an enteric polymer. Such a device would not release any drug in the stomach and when the device reaches the intestine, the enteric polymer is first dissolved and only then would the drug release begin. The drug release would take place in a sustained release fashion.

Enterically coated, osmotically activated devices can be manufactured using conventional materials, methods and equipment. For example, osmotically activated devices may be made by first encapsulating, in a pharmaceutically acceptable soft capsule, a liquid or semi-solid formulation of the compounds of the invention as described previously. This interior capsule is then coated with a semipermeable membrane composition (comprising, for example, cellulose acetate and polyethylene glycol 4000 in a suitable solvent such as a methylene chloride-methanol admixture), for example using an air suspension machine, until a sufficiently thick laminate is formed, e.g., around 0.05 mm. The semipermeable laminated capsule is then dried using conventional techniques. Then, an orifice having a desired diameter (e.g., about 0.99 mm) is provided through the semipermeable laminated capsule wall, using, for example, mechanical drilling, laser drilling, mechanical rupturing, or erosion of an erodible element such as a gelatin plug. The osmotically activated device may then be enterically coated as previously described. For osmotically activated devices containing a solid carrier rather than a liquid or semi-solid carrier, the interior capsule is optional; that is, the semipermeable membrane may be formed directly around the carrier-drug composition. However, preferred carriers for use in the drug-containing formulation of the osmotically activated device are solutions, suspensions, liquids, immiscible liquids, emulsions, sols, colloids, and oils. Particularly preferred carriers include, but are not limited to, those used for enterically coated capsules containing liquid or semisolid drug formulations.

Cellulose coatings include those of cellulose acetate phthalate and trimellitate; methacrylic acid copolymers, e.g. copolymers derived from methylacrylic acid and esters thereof, containing at least 40% methylacrylic acid; and especially hydroxypropyl methylcellulose phthalate. Methylacrylates include those of molecular weight above 100,000 daltons based on, e.g. methylacrylate and methyl or ethyl methylacrylate in a ratio of about 1:1. Typical products include Endragit L, e.g. L 100-55, marketed by Rohm GmbH, Darmstadt, Germany. Typical cellulose acetate phthalates have an acetyl content of 17-26% and a phthalate content of from 30-40% with a viscosity of ca. 45-90 cP. Typical cellulose acetate trimellitates have an acetyl content of 17-26%, a trimellityl content from 25-35% with a viscosity of ca. 15-20 cS. An example of a cellulose acetate trimellitate is the marketed product CAT (Eastman Kodak Company, USA). Hydroxypropyl methylcellulose phthalates typically have a molecular weight of from 20,000 to 130,000 daltons, a hydroxypropyl content of from 5 to 10%, a methoxy content of from 18 to 24% and a phthalyl content from 21 to 35%. An example of a cellulose acetate phthalate is the marketed product CAP (Eastman Kodak, Rochester N.Y., USA). Examples of hydroxypropyl methylcellulose phthalates are the marketed products having a hydroxypropyl content of from 6-10%, a methoxy content of from 20-24%, a phthalyl content of from 21-27%, a molecular weight of about 84,000 daltons, sold under the trademark HP50 and available from Shin-Etsu Chemical Co. Ltd., Tokyo, Japan, and having a hydroxypropyl content, a methoxyl content, and a phthalyl content of 5-9%, 18-22% and 27-35%, respectively, and a molecular weight of 78,000 daltons, known under the trademark HP55 and available from the same supplier.

The therapeutic agents may be provided in capsules, coated or not. The capsule material may be either hard or soft, and as will be appreciated by those skilled in the art, typically comprises a tasteless, easily administered and water soluble compound such as gelatin, starch or a cellulosic material. The capsules are preferably sealed, such as with gelatin bands or the like. See, for example, Remington: The Science and Practice of Pharmacy, Nineteenth Edition (Easton, Pa.: Mack Publishing Co., 1995), which describes materials and methods for preparing encapsulated pharmaceuticals.

A product containing therapeutic agent(s) of the invention can be configured as a suppository. The therapeutic agent(s) of the invention can be placed anywhere within or on the suppository to favorably affect the relative release of the therapeutic agent(s). The nature of the release can be zero order, first order, or sigmoidal, as desired.

Suppositories are solid dosage forms of medicine intended for administration via the rectum. Suppositories are compounded so as to melt, soften, or dissolve in the body cavity (around 98.6° F.) thereby releasing the medication contained therein. Suppository bases should be stable, nonirritating, chemically inert, and physiologically inert. Many commercially available suppositories contain oily or fatty base materials, such as cocoa butter, coconut oil, palm kernel oil, and palm oil, which often melt or deform at room temperature necessitating cool storage or other storage limitations. U.S. Pat. No. 4,837,214 to Tanaka et al. describes a suppository base comprised of 80 to 99 percent by weight of a lauric-type fat having a hydroxyl value of 20 or smaller and containing glycerides of fatty acids having 8 to 18 carbon atoms combined with 1 to 20 percent by weight diglycerides of fatty acids (which erucic acid is an example of). The shelf life of these type of suppositories is limited due to degradation. Other suppository bases contain alcohols, surfactants, and the like which raise the melting temperature but also can lead to poor absorption of the medicine and side effects due to irritation of the local mucous membranes (see for example, U.S. Pat. No. 6,099,853 to Hartelendy et al., U.S. Pat. No. 4,999,342 to Ahmad et al., and U.S. Pat. No. 4,765,978 to Abidi et al.).

The base used in the pharmaceutical suppository composition of this invention includes, in general, oils and fats comprising triglycerides as main components such as cacao butter, palm fat, palm kernel oil, coconut oil, fractionated coconut oil, lard and WITEPSOL®, waxes such as lanolin and reduced lanolin; hydrocarbons such as VASELINE®, squalene, squalane and liquid paraffin; long to medium chain fatty acids such as caprylic acid, lauric acid, stearic acid and oleic acid; higher alcohols such as lauryl alcohol, cetanol and stearyl alcohol; fatty acid esters such as butyl stearate and dilauryl malonate; medium to long chain carboxylic acid esters of glycerin such as triolein and tristearin; glycerin-substituted carboxylic acid esters such as glycerin acetoacetate; and polyethylene glycols and its derivatives such as macrogols and cetomacrogol. They may be used either singly or in combination of two or more. If desired, the composition of this invention may further include a surface-active agent, a coloring agent, etc., which are ordinarily used in suppositories.

The pharmaceutical composition of this invention may be prepared by uniformly mixing predetermined amounts of the active ingredient, the absorption aid and optionally the base, etc. in a stirrer or a grinding mill, if required at an elevated temperature. The resulting composition, may be formed into a suppository in unit dosage form by, for example, casting the mixture in a mold, or by forming it into a gelatin capsule using a capsule filling machine.

The compositions according to the present invention also can be administered as a nasal spray, nasal drop, suspension, gel, ointment, cream or powder. The administration of a composition can also include using a nasal tampon or a nasal sponge containing a composition of the present invention.

The nasal delivery systems that can be used with the present invention can take various forms including aqueous preparations, non-aqueous preparations and combinations thereof. Aqueous preparations include, for example, aqueous gels, aqueous suspensions, aqueous liposomal dispersions, aqueous emulsions, aqueous microemulsions and combinations thereof. Non-aqueous preparations include, for example, non-aqueous gels, non-aqueous suspensions, non-aqueous liposomal dispersions, non-aqueous emulsions, non-aqueous microemulsions and combinations thereof. The various forms of the nasal delivery systems can include a buffer to maintain pH, a pharmaceutically acceptable thickening agent and a humectant. The pH of the buffer can be selected to optimize the absorption of the therapeutic agent(s) across the nasal mucosa.

With respect to the non-aqueous nasal formulations, suitable forms of buffering agents can be selected such that when the formulation is delivered into the nasal cavity of a mammal, selected pH ranges are achieved therein upon contact with, e.g., a nasal mucosa. In the present invention, the pH of the compositions should be maintained from about 2.0 to about 6.0. It is desirable that the pH of the compositions is one which does not cause significant irritation to the nasal mucosa of a recipient upon administration.

The viscosity of the compositions of the present invention can be maintained at a desired level using a pharmaceutically acceptable thickening agent. Thickening agents that can be used in accordance with the present invention include methyl cellulose, xanthan gum, carboxymethyl cellulose, hydroxypropyl cellulose, carbomer, polyvinyl alcohol, alginates, acacia, chitosans and combinations thereof. The concentration of the thickening agent will depend upon the agent selected and the viscosity desired. Such agents can also be used in a powder formulation discussed above.

The compositions of the present invention can also include a humectant to reduce or prevent drying of the mucus membrane and to prevent irritation thereof. Suitable humectants that can be used in the present invention include sorbitol, mineral oil, vegetable oil and glycerol; soothing agents; membrane conditioners; sweeteners; and combinations thereof. The concentration of the humectant in the present compositions will vary depending upon the agent selected.

One or more therapeutic agents may be incorporated into the nasal delivery system or any other delivery system described herein.

A composition formulated for topical administration may be liquid or semi-solid (including, for example, a gel, lotion, emulsion, cream, ointment, spray or aerosol) or may be provided in combination with a “finite” carrier, for example, a non-spreading material that retains its form, including, for example, a patch, bioadhesive, dressing or bandage. It may be aqueous or non-aqueous; it may be formulated as a solution, emulsion, dispersion, a suspension or any other mixture.

Some modes of administration include topical application to the skin, eves or mucosa. Thus, typical vehicles are those suitable for pharmaceutical or cosmetic application to body surfaces. The compositions provided herein may be applied topically or locally to various areas in the body of a patient. As noted above, topical application is intended to refer to application to the tissue of an accessible body surface, such as, for example, the skin (the outer integument or covering) and the mucosa (the mucous-producing, secreting and/or containing surfaces). Exemplary mucosal surfaces include the mucosal surfaces of the eyes, mouth (such as the lips, tongue, gums, cheeks, sublingual and roof of the mouth), larynx, esophagus, bronchial, nasal passages, vagina and rectum/anus; in some embodiments, preferably the mouth, larynx, esophagus, vagina and rectum/anus; in other embodiments, preferably the eyes, larynx, esophagus, bronchial, nasal passages, and vagina and rectum/anus. As noted above, local application herein refers to application to a discrete internal area of the body, such as, for example, a joint, soft tissue area (such as muscle, tendon, ligaments, intraocular or other fleshy internal areas), or other internal area of the body. Thus, as used herein, local application refers to applications to discrete areas of the body.

With respect to topical and/or local administration of the present compositions, desirable efficacy may involve, for example, penetration of therapeutic agent(s) of the invention into the skin and/or tissue to substantially reach a hyperalgesic site to provide desirable anti-hyperalgesic pain relief. The efficacy of the present compositions may be about the same as that achieved, for example, with central opiate analgesics. But, as discussed in detail herein, the efficacy achieved with therapeutic agent(s) of the invention is preferably obtained without the undesirable effects that are typically associated with central opiates including, for example, respiratory depression, sedation, and addiction, as it is believed that therapeutic agent(s) of the invention does not cross the blood brain barrier.

Also in certain embodiments, including embodiments that involve aqueous vehicles, the compositions may also contain a glycol, that is, a compound containing two or more hydroxy groups. A glycol which is particularly preferred for use in the compositions is propylene glycol. In these embodiments, the glycol is preferably included in the compositions in a concentration of from greater than 0 to about 5 wt. %, based on the total weight of the composition. More preferably, the compositions contain from about 0.1 to less than about 5 wt. % of a glycol, with from about 0.5 to about 2 wt. % being even more preferred. Still more preferably, the compositions contain about 1 wt. % of a glycol.

For local internal administration, such as intra-articular administration, the compositions are preferably formulated as a solution or a suspension in an aqueous-based medium, such as isotonically buffered saline or are combined with a biocompatible support or bioadhesive intended for internal administration.

Lotions, which, for example, may be in the form of a suspension, dispersion or emulsion, contain an effective concentration of one or more of the compounds. The effective concentration is preferably to deliver an effective amount, typically at a concentration of between about 0.1-50% [by weight] or more of one or more of the compounds provided herein. The lotions also contain [by weight] from 1% to 50% of an emollient and the balance water, a suitable buffer, and other agents as described above. Any emollients known to those of skill in the art as suitable for application to human skin may be used. These include, but are not limited to, the following: (a) Hydrocarbon oils and waxes, including mineral oil, petrolatum, paraffin, ceresin, ozokerite, microcrystalline wax, polyethylene, and perhydrosqualene. b) Silicone oils, including dimethylpolysiloxanes, methylphenylpolysiloxanes, water-soluble and alcohol-soluble silicone-glycol copolymers. (c) Triglyceride fats and oils, including those derived from vegetable, animal and marine sources. Examples include, but are not limited to, castor oil, safflower oil, cotton seed oil, corn oil, olive oil, cod liver oil, almond oil, avocado oil, palm oil, sesame oil, and soybean oil. (d) Acetoglyceride esters, such as acetylated monoglycerides. (e) Ethoxylated glycerides, such as ethoxylated glyceryl monostearate. (f) Alkyl esters of fatty acids having 10 to 20 carbon atoms. Methyl, isopropyl and butyl esters of fatty acids are useful herein. Examples include, but are not limited to, hexyl laurate, isohexyl laurate, isohexyl palmitate, isopropyl palmitate, isopropyl myristate, decyl oleate, isodecyl oleate, hexadecyl stearate, decyl stearate, isopropyl isostearate, diisopropyl adipate, diisohexyl adipate, dihexyldecyl adipate, diusopropyl sebacate, lauryl lactate, myristyl lactate, and cetyl lactate. (g) Alkenyl esters of fatty acids having 10 to 20 carbon atoms. Examples thereof include, but are not limited to, oleyl myristate, oleyl stearate, and oleyl oleate. (h) Fatty acids having 9 to 22 carbon atoms. Suitable examples include, but are not limited to, pelargyonic, lauric, myristic, palmitic, stearic, isostearic, hydroxystearic, oleic, linoleic, ricinoleic, arachidonic, behenic, and erucic acids. (i) Fatty alcohols having 10 to 22 carbon atoms, such as, but not limited to, lauryl, myristyl, cetyl, hexadecyl, stearyl, isostearyl, hydroxystearyl, oleyl, ricinoleyl, behenyl, erucyl, and 2-octyl dodecyl alcohols. (j) Fatty alcohol ethers, including, but not limited to ethoxylated fatty alcohols of 10 to 20 carbon atoms, such as, but are not limited to, the lauryl, cetyl, stearyl, isostearyl, oleyl, and cholesterol alcohols having attached thereto from 1 to 50 ethylene oxide groups or 1 to 50 propylene oxide groups or mixtures thereof. (k) Ether-esters, such as fatty acid esters of ethoxylated fatty alcohols. (l) Lanolin and derivatives, including, but not limited to, lanolin, lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin, acetylated lanolin alcohols, lanolin alcohols linoleate, lanolin alcohols ricinoleate, acetate of lanolin alcohols ricinoleate, acetate of ethoxylated alcohol(S)-esters, hydrogenolysis of lanolin, ethoxylated hydrogenated lanolin, ethoxylated sorbitol lanolin, and liquid and semisolid lanolin absorption bases. (m) polyhydric alcohols and polyether derivatives, including, but not limited to, propylene glycol, dipropylene glycol, polypropylene glycol [M.W. 2000-4000], polyoxyethylene polyoxypropylene glycols, polyoxypropylene polyoxyethylene glycols, glycerol, ethoxylated glycerol, propoxylated glycerol, sorbitol, ethoxylated sorbitol, hydroxypropyl sorbitol, polyethylene glycol [M.W. 200-6000], methoxy polyethylene glycols 350, 550, 750, 2000, 5000, poly(ethylene oxide) homopolymers [M.W. 100,000-5,000,000], polyalkylene glycols and derivatives, hexylene glycol (2-methyl-2,4-pentanediol), 1,3-butylene glycol, 1,2,6,-hexanetriol, ethohexadiol USP (2-ethyl-1,3-hexanediol), C₁₅-C₁₈ vicinal glycol and polyoxypropylene derivatives of trimethylolpropane. (n) polyhydric alcohol esters, including, but not limited to, ethylene glycol mono- and di-fatty acid esters, diethylene glycol mono- and di-fatty acid esters, polyethylene glycol [M.W. 200-6000], mono- and di-fatty esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters. (o) Wax esters, including, but not limited to, beeswax, spermaceti, myristyl myrstate, and stearyl stearate and beeswax derivatives, including, but not limited to, polyoxyethylene sorbitol beeswax, which are reaction products of beeswax with ethoxylated sorbitol of varying ethylene oxide content that form a mixture of ether-esters. (p) Vegetable waxes, including, but not limited to, carnauba and candelilla waxes. (q) phospholipids, such as lecithin and derivatives. (r) Sterols, including, but not limited to, cholesterol and cholesterol fatty acid esters. (s) Amides, such as fatty acid amides, ethoxylated fatty acid amides, and solid fatty acid alkanolamides.

The lotions further preferably contain [by weight] from 1% to 10%, more preferably from 2% to 5%, of an emulsifier. The emulsifiers can be nonionic, anionic or cationic. Examples of satisfactory nonionic emulsifiers include, but are not limited to, fatty alcohols having 10 to 20 carbon atoms, fatty alcohols having 10 to 20 carbon atoms condensed with 2 to 20 moles of ethylene oxide or propylene oxide, alkyl phenols with 6 to 12 carbon atoms in the alkyl chain condensed with 2 to 20 moles of ethylene oxide, mono- and di-fatty acid esters of ethylene oxide, mono- and di-fatty acid esters of ethylene glycol where the fatty acid moiety contains from 10 to 20 carbon atoms, diethylene glycol, polyethylene glycols of molecular weight 200 to 6000, propylene glycols of molecular weight 200 to 3000, glycerol, sorbitol, sorbitan, polyoxyethylene sorbitol, polyoxyethylene sorbitan and hydrophilic wax esters. Suitable anionic emulsifiers include, but are not limited to, the fatty acid soaps, e.g., sodium, potassium and triethanolamine soaps, where the fatty acid moiety contains from 10 to 20 carbon atoms. Other suitable anionic emulsifiers include, but are not limited to, the alkali metal, ammonium or substituted ammonium alkyl sulfates, alkyl arylsulfonates, and alkyl ethoxy ether sulfonates having 10 to 30 carbon atoms in the alkyl moiety. The alkyl ethoxy ether sulfonates contain from 1 to 50 ethylene oxide units. Among satisfactory cationic emulsifiers are quaternary ammonium, morpholinium and pyridinium compounds. Certain of the emollients described in preceding paragraphs also have emulsifying properties. When a lotion is formulated containing such an emollient, an additional emulsifier is not needed, though it can be included in the composition.

The balance of the lotion is water or a C7 or C3 alcohol, or a mixture of water and the alcohol. The lotions are formulated by simply admixing all of the components together. Preferably the compound, such as loperamide, is dissolved, suspended or otherwise uniformly dispersed in the mixture.

Other conventional components of such lotions may be included. One such additive is a thickening agent at a level from 1% to 10% by weight of the composition. Examples of suitable thickening agents include, but are not limited to: cross-linked carboxypolymethylene polymers, ethyl cellulose, polyethylene glycols, gum tragacanth, gum kharaya, xanthan gums and bentonite, hydroxyethyl cellulose, and hydroxypropyl cellulose.

Creams can be formulated to contain a concentration effective to deliver an effective amount of therapeutic agent(s) of the invention to the treated tissue, typically at between about 0.1%, preferably at greater than 1% up to and greater than 50%, preferably between about 3% and 50%, more preferably between about 5% and 15% therapeutic agent(s) of the invention. The creams also contain from 5% to 50%, preferably from 10% to 25%, of an emollient and the remainder is water or other suitable non-toxic carrier, such as an isotonic buffer. The emollients, as described above for the lotions, can also be used in the cream compositions. The cream may also contain a suitable emulsifier, as described above. The emulsifier is included in the composition at a level from 3% to 50%, preferably from 5% to 20%.

These compositions that are formulated as solutions or suspensions may be applied to the skin, or, may be formulated as an aerosol or foam and applied to the skin as a spray-on. The aerosol compositions typically contain [by weight] from 25% to 80%, preferably from 30% to 50%, of a suitable propellant. Examples of such propellants are the chlorinated, fluorinated and chlorofluorinated lower molecular weight hydrocarbons. Nitrous oxide, carbon dioxide, butane, and propane are also used as propellant gases. These propellants are used as understood in the art in a quantity and under a pressure suitable to expel the contents of the container.

Suitably prepared solutions and suspensions may also be topically applied to the eyes and mucosa. Solutions, particularly those intended for ophthalmic use, may be formulated as 0.01%-10% isotonic solutions, pH about 5-7, with appropriate salts, and preferably containing one or more of the compounds herein at a concentration of about 0.11%, preferably greater than 1%, up to 50% or more. Suitable ophthalmic solutions are known [see, e.g., U.S. Pat. No. 5,116,868, which describes typical compositions of ophthalmic irrigation solutions and solutions for topical application]. Such solutions, which have a pH adjusted to about 7.4, contain, for example, 90-100 mM sodium chloride, 4-6 mM dibasic potassium phosphate, 4-6 mM dibasic sodium phosphate, 8-12 mM sodium citrate, 0.5-1.5 mM magnesium chloride, 1.5-2.5 mM calcium chloride, 15-25 mM sodium acetate, 10-20 mM D.L.-sodium, β-hydroxybutyrate and 5-5.5 mM glucose.

Gel compositions can be formulated by simply admixing a suitable thickening agent to the previously described solution or suspension compositions. Examples of suitable thickening agents have been previously described with respect to the lotions.

The gelled compositions contain an effective amount of therapeutic agent(s) of the invention, typically at a concentration of between about 0.1-50% by weight or more of one or more of the compounds provided herein.; from 5% to 75%, preferably from 10% to 50%, of an organic solvent as previously described; from 0.5% to 20%, preferably from 1% to 10% of the thickening agent; the balance being water or other aqueous or non-aqueous carrier, such as, for example, an organic liquid, or a mixture of carriers.

The formulations can be constructed and arranged to create steady state plasma levels. Steady state plasma concentrations can be measured using HPLC techniques, as are known to those of skill in the art. Steady state is achieved when the rate of drug availability is equal to the rate of drug elimination from the circulation. In typical therapeutic settings, the therapeutic agent(s) of the invention will be administered to patients either on a periodic dosing regimen or with a constant infusion regimen. The concentration of drug in the plasma will tend to rise immediately after the onset of administration and will tend to fall over time as the drug is eliminated from the circulation by means of distribution into cells and tissues, by metabolism or by excretion. Steady state will be obtained when the mean drug concentration remains constant over time. In the case of intermittent dosing, the pattern of the drug concentration cycle is repeated identically in each interval between doses with the mean concentration remaining constant. In the case of constant infusion, the mean drug concentration will remain constant with very little oscillation. The achievement of steady state is determined by means of measuring the concentration of drug in plasma over at least one cycle of dosing such that one can verify that the cycle is being repeated identically from dose to dose. Typically, in an intermittent dosing regimen, maintenance of steady state can be verified by determining drug concentrations at the consecutive troughs of a cycle, just prior to administration of another dose. In a constant infusion regimen where oscillation in the concentration is low, steady state can be verified by any two consecutive measurements of drug concentration.

To improve oral bioavailability of the compounds of the present invention, excipients may be used that increase intestinal membrane permeability (Aungst, B. J. J Pharmaceutical Science Vol. 89, Issue 4, pp. 429-442, 2000). Permeation enhancers may include surfactants, fatty acids, medium chain glycerides, steroidal detergents, acyl carnitine and alkanoylcholines, N-acetylated alpha-amino acids and N-acetylated non-alpha-amino acids, and chitosans, and other mucoadhesive polymers. Specific examples include: cholate glycocholate, glycosursodeoxycholate, ethylenediaminetetraacetic acid, hydroxypropyl-beta-cyclodextrin, hydroxypropyl-gamma-cylcodextrin, gamma-cylcodextrin, tetradecyl-beta-D-maltose, octylglucoside, citric acid, glycyrrhetinic acid, and Tween-80® (Shah, R. B. et al J. Pharm. Sci Apr 93(4): 1070-82, 2004).

The (S)-7,8-saturated-4,epoxy-morphinanium of the present invention may be supplied in kit form. The kit includes a vial containing (S)-7,8-saturated-4,5-epoxy-morphinanium compound tablets. The kit also includes instructions for administering the tablets to a subject, for example, to a patient who has diarrhea or who has symptoms of diarrhea. The instructions include indicia, for example writing, indicating that the (S)-7,8-saturated-4,5-epoxy-morphinanium is pure (S)-7,8-saturated-4,5-epoxy-morphinanium free of its counterpart (R)-7,8-saturated-4,5-epoxy-morphinanium.

In some embodiments of the invention, the kit can include optionally or alternatively a pharmaceutical preparation vial and a pharmaceutical preparation diluent vial. The vial containing the diluent for the pharmaceutical preparation is optional. The diluent vial contains a diluent such as physiological saline for diluting what could be a concentrated solution or lyophilized powder of (S)-7,8-saturated-4,5-epoxy-morphinanium. The instructions can include instructions for mixing a particular amount of the diluent with a particular amount of the concentrated pharmaceutical preparation whereby a final formulation for injection or infusion is prepared. The instructions can include instructions for treating a patient with an effective amount of (S)-7,8-saturated-4,5-epoxy-morphinanium. It also will be understood that the containers containing the preparations, whether the container is a bottle, a vial with a septum, an ampoule with a septum, an infusion bag, and the like, can contain additional indicia such as conventional markings which change color when the preparation has been autoclaved or otherwise sterilized.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### Example I

### Synthesis and isolation of (S)-17-(3′-phenylbut-2′-ynyl)-4,5α-epoxy-3,14-di-hydroxy-17-methyl-6-oxomorphinanium iodide

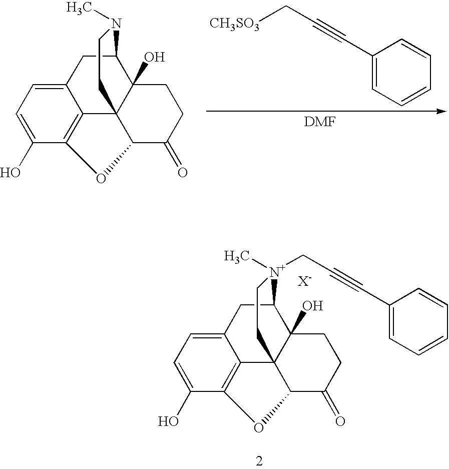

Oxymorphone (200 mg, 0.66 mmol) and 3-phenylpropargyl mesylate (209 mg, 0.997 mmol) were dissolved in 1 mL of dimethylformamide. The reaction was stirred overnight on a steam bath. HPLC analysis showed 54% product, 13% oxymorphone, and several unknown impurities (33% combined). The reaction was stripped, dissolved in ethanol (1 mL), stored in a freezer overnight and stripped again. The residue was partitioned between water and 20% isopropanol in chloroform. The layers were separated and the aqueous layer was treated with 1 ml of a 10% solution of sodium iodide. The aqueous phase was extracted with 20% isopropanol in chloroform. The organic phase was filtered through 1 PS paper and the solvent removed in vacuo and the residue was portioned between water and 20% isopropanol chloroform and the layers were separated. The aqueous phase was treated with 200 mg of sodium iodide and re-extracted with 20% isopropanol chloroform. The organic phases were combined, filtered through 1 PS paper and stripped on a rotary evaporator to give 100 mg of residue. The residue was then purified by column chromatography (Biotage 25M silica gel column) eluting with 650 mL of a linear gradient of 0-20% methanol in methylene chloride. The purest product containing fractions were combined and stripped to give 50 mg of product (18% yield).

¹H NMR (300 MHz, CD₃OD) δ 7.7-7.4 (m, 5H), 6.79 (s, 2H), 5.99 (d, J=15.9, 1H), 4.93 (d, J=15.9, 1H), 4.92 (s, 1H), 4.27 (d, J=4.2, 1H), 3.7-3.6 (m, 2H), 3.45 (s, 3H), 3.4-3.1 (m, 2H), 3.1-2.9 (m, 2H), 2.25 (dt, J=15, 3, 1H), 2.2-2.1 (m, 1H), 1.9-1.8 (m, 2H). MS [M⁺]: 417.2. HPLC purity: 95.9% (UV detection at 280 nm).

HPLC analysis showed the purity to be >95%.

HPLC conditions: Hewlett Packard 1100 series; Column: Phenomonex Synergi hydro RP column (C18, 5μ, 150×4.6 mm); Flow rate: 1.0 mL/min, Column temperature: 40° C.; Detector: diode array detector monitoring @ 220 and 210 nm; Elution: isocratic. 60% water, 30% buffer*, 10% methanol; * 700 ml of water, 300 mL methanol, 3 mL triethylamine and sufficient phosphoric acid to give a pH of 3.4; or alternatively: Column: Phenomonex Synergi hydro RP column (C18, 5μ, 150×4.6 mm); Flow rate: 1.5 mL/min; Column temperature: 50° C.; Detector: diode array detector monitoring @ 220 and 280 nm; Elution: gradient.

| Time min | Methanol | Water | Mix^{a} | Curve |
|---|---|---|---|---|
| 0 | 0% | 90% | 10% | initial |
| 45 | 30% | 60% | 10% | Linear |
| 45.1 | 0% | 90% | 10% | Linear |
| 50 | 0% | 90% | 10% | Hold |

^{a}(49.5% water, 49.5% methanol, 1% trifuoroacetic acid)

### Example II

Overview. Anhydrous reactions were carried out in oven dried glassware under an atmosphere of nitrogen. Naltrexone and Nalmefene were purchased from Mallinkrodt as their HCl salts and were free based prior to use by washing with sodium bicarbonate solution. Methyl iodide was purchased from Alfa Aesar. All the solvents were purchased from Aldrich Co. Chemicals from commercial sources were used as received. Purification of the quaternary compounds was performed on a CombiFlash™Sq16x from ISCO Inc. using a 4.3 g Reverse Phase (C18) RediSep column which has been reused. The analytical HPLC was performed on a Phenomenex Prodigy 5 μm ODS3 100 A column (150×4.6 mm) and purification was performed on a semi-prep Phenomenex Prodigy 5 μm ODS3 100 A column (250×21.2 mm). NMR spectra were recorded on a JEOL 300 MHz spectrometer. HPLC and MS data were obtained on an Agilent series 1100/1200 LC/MSD system.

### (S)-17-Allyl-17-cyclopropylmethyl-4,5α-epoxy-3,14-di-hydroxy-6-oxomorphinanium Iodide

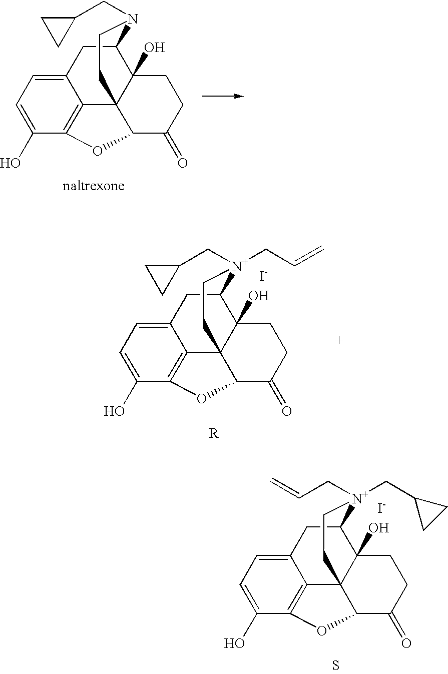

Synthetic Procedure. Naltrexone (2.0 g, 5.86 mmol) was dissolved in DMF (10 mL, anhydrous) under nitrogen. Allyl iodide (0.5 mL, 5.18 mmol) was added. The mixture was stirred at room temperature for 4 days. DMF was removed. The residue was stirred with 50 mL of water for 10 min. The aqueous solution was separated from the solid precipitates and washed with dichloromethane (50 mL). It was lyophilized to give a hygroscopic solid (1.2 g). 0.2 g of this solid was dissolved in water (30 mL). The pH of the water solution was adjusted to 10 by Na₂CO₃. This solution was washed with dichloromethane (2×20 mL) and lyophilized to give a yellow solid. This solid was purified by a reverse phase column (4 g, C18) to 28 mg of a solid which was later identified as a mixture of F 27-R and F27-S. The remaining of the above hygroscopic solid (˜1.0 g) was subjected to the same treatments to give another 81 mg solid as a mixture of F 27-R and F27-S. This 81 mg solid was separated by semi-prep HPLC to give 55 mg (2%) of (R) and 9.5 mg (0.3%) of S.

R: ¹H NMR (300 MHz, D₂O) δ 6.83 (d, J=8.4 Hz, 1H), 6.77 (d, J=8.4 Hz, 1H), 6.14-6.04 (m, 1H), 5.73-5.67 (m, 1H), 5.13-5.04 (m, 1H), 5.04 (s, 1H), 4.97-4.89 (m, 1H), 3.72-3.58 (m, 3H), 3.17-2.83 (m, 5H), 2.30-2.25 (m, 1H), 2.16-2.09 (m, 1H), 1.88-1.78 (m, 1H), 1.24-1.14 (m, 1H), 0.85-0.75 (m, 2H), 0.52-0.42 (m, 2H). MS [M⁺]: 382.2. HPLC purity: 99% (UV detection at 254 nm).

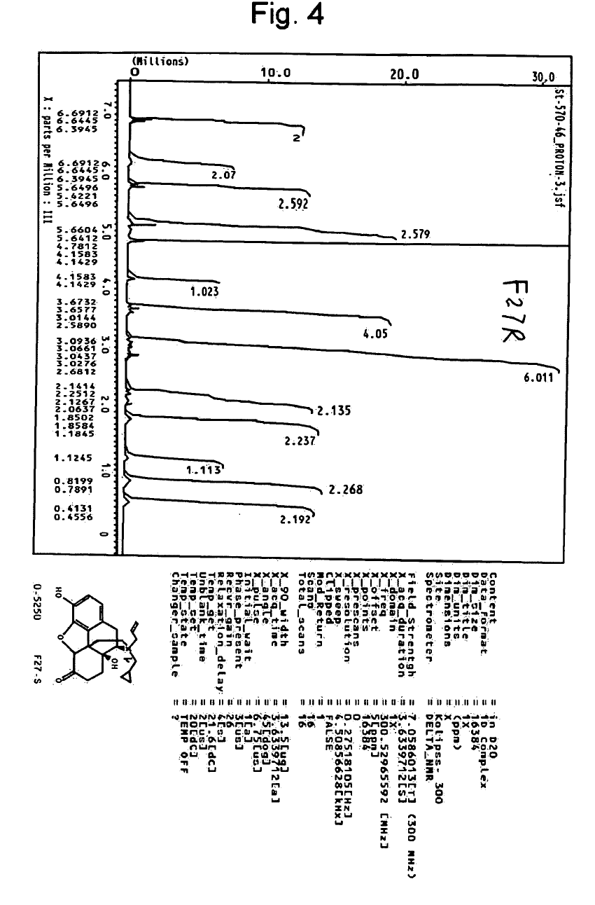

FIG. 4 is a proton NMR spectrum of (R)-17-allyl-17-cyclopropylmethyl-4,5α-epoxy-3,14-dihydroxy-6-oxomorphinanium iodide.

S: ¹H NMR (300 MHz, D₂O) δ 6.67 (d, J=8.4 Hz, 1H), 6.39 (d, J=8.4 Hz, 1H), 6.64 (m, 1H), 5.5.42 (m, 2H), 5.05 (s, 1H), 4.8 (m, 2H), 3.68 (m, 2H), 3.17 (m, 1H), 2.90 (m, 4H), 2.40 (m, 1H), 2.16 (m, 4H), 1.70 (m, 1H), 0.83 (m, 1H), 0.58 (m, 2H), 0.21 (m, 2H). MS [M⁺]: 382.2. HPLC purity: 99% (UV detection at 254 nm).

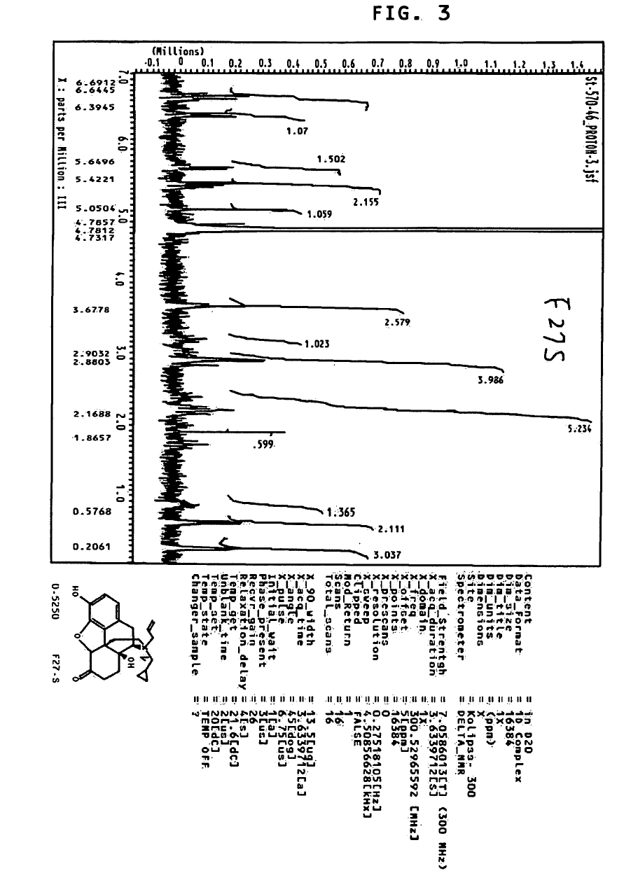

FIG. 3 is a proton NMR spectrum of (S)-17-allyl-17-cyclopropylmethyl-4,5α-epoxy-3,14-dihydroxy-6-oxomorphinanium iodide.

Opiate Receptor Binding of (S)-7,8-saturated-4,5α-epoxy-morphinaniums. Radioligand binding assays may be conducted to determine the binding specificity of an (S)-7,8-saturated-4,5-epoxy-morphinanium for μ-, κ-, and δ-opiate receptors using methods adapted from scientific literature (Simonin, F et al 1994, Mol. Pharmacol. 46:1015-1021; Maguire, P. et al 1992, Eur. J. Pharmacol. 213:219-225; Simonin, F. et al PNAS USA 92(15):1431-1437; Wang, J B 1994, FEBS Lett 338:217-222). For example, a membrane may be associated with human opioid receptor material. Diprenorphine which has an affinity for all four opioid receptors, can be used as a competitive challenge to the test compound. Membranes can then be separated, and the binding of the test compounds to the receptor material can be determined by scintillation counting. A control, such as naltrexone, can be used to determine relative binding affinity.

(S)-17-allyl-17-cyclopropylmethyl-4,5α-epoxy-3,14-di-hydroxy-6-oxomorphinanium iodide was found to display 68% inhibition of the μ receptors compared to naltrexone control. (S)-17-(3′-phyenylbut-2′-ynyl)-4,5α-epoxy-3,14-di-hydroxy-17-methyl-6-oxomorphinanium iodide demonstrated 80% inhibition with respect to control specific binding at the t receptor. (S)-17-(3,3-dimethylallyl)-4,5α-epoxy-3,14-di-hydroxy-17-methyl-6-oxomorphinanium iodide demonstrated 65% inhibition of control g (naltrexone) specific binding.

In Vitro Pharmacology of (S)-7,8-saturated-4,5α-epoxy-morphinaniums on μ receptor. Mμ-receptor agonist/antagonist activity may be adjudged by use of field-stimulated guinea pig ileum by methods known in the art. For example, segments of guinea pig terminal ileum may be suspended in 20-ml organ baths filled with an oxygenated (95% O₂ and 5% CO₂) and pre-warmed (37° C.) physiological salt solution of the following composition (in mM): NaCl 118.0, KCl 4.7, MgSO₄ 1.2, CaCl₂ 2.5, KH₂PO₄ 1.2, NaHCO₃ 25.0 and glucose 11.0 (pH 7.4). Additional experimental conditions that may be followed are described in Hutchinson et al. (1975) Brit. J. Pharmacol., 55:541-546.

Indomethacin (1 μM), nor-binaltorphimine (0.01 μM), methysergide (1 μM), ondansetron (10 μM) and GR113808 (0.1 μM) may be also present throughout an experiment to prevent prostanoid release and to block the k-opioid, 5-HT2, 5-HT3 and 5-HT4 receptors, respectively. The tissues in such tests are typically connected to force transducers for isometric tension recordings. The tissue may be stretched to a resting tension, for example, of 1 g then allowed to equilibrate, for example, about 60 min during which time they may be washed repeatedly and the tension readjusted. Electrical stimulation with pulses of minimal intensity to trigger maximal contractions and a short duration, for example, 1 ms duration, delivered by a constant current stimulator at a frequency such a 0.1 Hz. The experiments may be carried out using a semi-automated isolated organ system possessing multi-organ baths, with multichannel data acquisition.

Exemplar Test for Agonist Activity. The tissues may be exposed to a submaximal concentration of the reference agonist DAMGO (0.1 μM) to verify responsiveness and to obtain a control response. Following extensive washings and recovery of the control twitch contractions, the tissues may be exposed to increasing concentrations of the (S)-7,8-saturated-4,5-epoxy-morphinanium or the same agonist. The different concentrations may be added cumulatively and each left in contact with the tissues until a stable response is obtained or for a maximum of 15 min. If an agonist-like response (inhibition of twitch contractions) is obtained, the reference antagonist naloxone (0.1 μM) may be tested against the highest concentration of the (S)-7,8-saturated-4,5-epoxy-morphinanium used to confirm the involvement of the is receptors in the response.

Exemplary Test for Antagonist Activity. The tissues may be exposed to a submaximal concentration of the reference agonist DAMGO (0.1 μM) to obtain a control response. After stabilization of the DAMGO-induced response, increasing concentrations of an (S)-7,8-saturated-4,5-epoxy-morphinanium or the reference antagonist naloxone may be added cumulatively. Each concentration may be left in contact with the tissues until a stable response is obtained or for a maximum time, such as 15 min. The maximum change in the amplitude of the electrically-evoked twitch contractions induced by each compound concentration may be measured. Results may be expressed as a percent of the control response to DAMGO (mean values). The EC₅₀ value (concentration producing a half-maximum response) or IC₅₀ value (concentration causing a half-maximum inhibition of the response to DAMGO) may be determined by linear regression analysis of the concentration-response curves. Inhibition of the DAMGO-induced response by the (S)-7,8-saturated-4,5-epoxy-morphinanium may indicate an antagonist activity at the μ receptors.

In a field-stimulated guinea pig ileum, the μ receptor agonist DAMGO induces a concentration-dependent decrease in the twitch contraction amplitude which is reversed by the antagonist naloxone in a concentration-dependent manner. In the untreated tissues, an agonist causes a concentration-dependent and naloxone-sensitive decrease in the twitch contraction amplitude. In tissues previously depressed with DAMGO, an agonist does not produce any recovery of the twitch contraction amplitude but causes a further decrease.

### Example III

Effects of (S)-17-(3′-phenylbut-2′ynyl)-4.5α-epoxy-3,14-di-hydroxy-17-methyl-6-oxomorphinanium iodide (“(S)-PM”)
and (S)-17-(3,3-dimethylallyl)-4,5α-epoxy-3,14-di-hydroxy-17-methyl-6-oxomorphinanium iodide (“(S)-DMAM”)
evaluated for agonist and antagonist activities at the μ-opioid receptors in the guinea pig ileum
Evaluation of agonist activity

| | Control | | | | | | | | | | + |
|---|---|---|---|---|---|---|---|---|---|---|---|
| | response to | Responses to increasing concentrations of the compounds | | | | | | | | | Naloxone |
| | DAMGO | (M) | | | | | | | | | (1.0E−07 M) |
| Compounds | (1.OE-07 M) | 1.0E−08 | 3.0E−08 | 1.0E−07 | 3.0E−07 | 1.0E−06 | 3.0E−06 | 1.0E−05 | 3.0E−05 | 1.0E−04 | 1.0E−04 M |
| (S)-PM | 100 | 0 | 0 | 6 | 12 | 25 | 38 | 53 | 71 | 89 | 30 |
| (S)-DMAM | 100 | 0 | 0 | 8 | 19 | 38 | 77 | 93 | 99 | 101 | 77 |
| | | 1.0E−09 | | | 1.0E−08 | | | 1.0E−07 | | 1.0E−06 | |
| DAMGO | 100 | 9 | | | 57 | | | 96 | | 103 | 4 |

Evaluation of antagonist activity

| | Control | Responses to DAMGO (1.0E−07 M) in the presence of | | | | | | | | |
|---|---|---|---|---|---|---|---|---|---|---|
| | response to | increasing concentrations of the compounds | | | | | | | | |
| | DAMGO | (M) | | | | | | | | |
| Compounds | (1.0E−07 M) | 1.0E−08 | 3.0E−08 | 1.0E−07 | 3.0E−07 | 1.0E−06 | 3.0E−06 | 1.0E−05 | 3.0E−05 | 1.0E−04 |
| (S)-PM | 100 | 100 | 100 | 100 | 100 | 100 | 100 | 100 | 100 | 110 |
| (S)-DMAM | 100 | 100 | 100 | 100 | 100 | 101 | 105 | 110 | 113 | 113 |
| | | 5.0E−09 | | | 2.0E−08 | | | 1.0E−07 | | |
| Naloxone | 100 | 85 | | | 51 | | | −6 | | |

The results are expressed as a percent of the control response to DAMGO (decrease in twitch contraction amplitude) (mean values; n=2)

EC₅₀ and IC₅₀ values determined for (S)-17-(3′-phenylbut-2′ynyl)-
4.5α-epoxy-3,14-di-hydroxy-17-methyl-6-oxomorphinanium iodide
(“(S)-PM”) and (S)-17-(3,3-dimethylallyl-dihydroxy-17-methyl-
6-oxomorphinanium oxide (“(S)-DMAM”) at theμ-opioid
receptors in the guinea pig ileum

| | Agonist activity | Antagonist activity |
|---|---|---|
| Compound | EC₅₀ value | IC₅₀ value |
| (S)-PM | 6.8.0E-06 M | No antagonist activity |
| (S)-DMAM- | 1.3e-06 M | No antagonist activity |

Gastrointestinal Transit in Rats Test. The effect of the (S)-N-7,8-saturated-4,5-epoxy-morphinaniums of the present invention on morphine-induced inhibition of gastrointestinal transit in rats may be determined using methods known in the art, including that described in A. F. Green, Br. J. Pharmacol. 14: 26-34, 1959; L. B. Witkin, C. F. et al J. Pharmacol. Exptl. Therap. 133: 400-408, 1961; D. E. Gmerek, et al J. Pharmacol. Exptl. Ther. 236: 8-13, 1986; and O. Yamamoto et al. Neurogastroenterol. Motil. 10: 523-532, 1998. Such tests may be used to demonstrate usefulness of (S)-agonists in the treatment of intestinal hypermotility problems.

In such test, the compound is administered subcutaneously to rats at increasing concentrations. A 10% suspension of activated charcoal in 0.25% methylcellulose is administered orally after the subcutaneous dose of the control (e.g., morphine) and the test agonist compound. Rats are euthanized after receiving the charcoal and the intestines removed and lightly stretched on moist paper along a meterstick. The small intestine from pyloric sphincter to caecum is measured and the distance traveled by the charcoal as a fraction of that length is evaluated for each rat. The individual distance traveled by the charcoal in centimeters was divided by the total length of the intestines in centimeters (pyloric sphincter to caecum) for each rat.

Tests for Anti-Diarrheal Activity. Tests for antidiarrheal activity may also be run for (S)-N-7,8-saturated-4,5-epoxy-morphinaniums of the present invention. For example, the castor oil tests described in Niemegeers et al. (1972) Arzneim Forsch 22:516-518; U.S. Pat. Nos. 4,867,979; 4,990,521; 4,824,853 may be used. In such tests, rats or mice may be fasted overnight. Each animal is treated intravenously with the desired dose of the compound to be tested. A period of time thereafter, the animal receives a dose of oil, such as castor oil or ricinio oil, orally. Each animal is kept in an individual cage. A period of time after the castor oil treatment, each animal is assessed for the presence or absence of diarrhea. The ED₅₀ value is determined as that dose in mg/kg body weight at which no diarrhea is present in 50% of the tested animals.

Anti-diarrheal activity can also be determined by assessing the effects of a compound as an antagonist of PGE₂-induced diarrhea in mice [see, e.g., Dajani et al. 1975) European Jour. Pharmacol. 34:105-113; and Dajani et al. (1977) J. Pharmacol. Exp. Ther. 203:512-526; see, e.g., U.S. Pat. No. 4,870,084]. This method reliably elicits diarrhea in otherwise untreated mice within 15 minutes.

Analgesic Activity of Tests. The following pain models are useful in determining the analgesic activity of an (S)-N-7,8-saturated-4,5-epoxy-morphinanium:

Acetic Acid Writhing assay in Mice. Mice (CD-1, male) are weighed and placed in individual squares. The test or control article are administered and after the appropriate absorption time, acetic acid solution are administered intraperitoneally. Ten minutes after the i.p. injection of acetic acid, the number of writhes are recorded for a period of 5 minutes.

The total number of writhes for each mouse are recorded. The mean number of writhes for the control and each test article group are compared using an ANOVA followed by a relevant multiple comparison test and percent inhibition calculated.

Phenylquinone (PPQ) Writhing Assay. Mice (CD-1, male) are weighed and placed in individual squares. The test or control article are administered and after the appropriate absorption time, the PPQ solution (0.02% aqueous solution) is administered intraperitoneally. Each animal is observed closely for ten minutes for exhibition of writhing.

The total number of writhes for each mouse are recorded. The mean number of writhes for the control and each test article group are compared using an ANOVA followed by a relevant multiple comparison test and percent inhibition calculated.

Randall-Selitto Assay in Rats. The purpose of this assay is to determine the effect of test articles upon the pain threshold of rats.

Following an overnight fast, rats are placed in groups of ten. Twenty rats are used as vehicle controls. The rats are then sequentially injected with a 20% Brewer's yeast suspension into the plantar surface of the left hind paw. Two hours later the rats are administered the test article, reference drug, or control vehicle. One hour after dose administration, the pain threshold of the inflamed and non-inflamed paw is measured by a “Analgesia Meter” that exerts a force which increases at a constant rate along a linear scale.

The control group threshold and standard deviation for the inflamed paw and non-inflamed paw are calculated. Rats in the test article group and reference group are considered protected if the individual pain threshold exceeds the control group mean threshold by two standard deviations of the means.

Hot Plate Analgesia Assay. Each mouse (CD-1, male) serves as its own control throughout the experiment. The mice are placed sequentially on a Hot Plate Analgesia Meter (set for 55° C.±2° C.). The mice react characteristically to the heat stimulus by:
1. Licking the forepaw
2. Rapid fanning of the hind paw
3. A sudden jump off the hot plate

Any of the three types of reactions are taken as an end point to the heat stimulus. The mouse is removed from the hot plate immediately upon displaying the end point. The reaction time is measured quantitatively by the number of seconds that elapse between the placing of the mouse on the hot plate and the display of a definitive end point. Elapsed time is measured by a stop watch accurate to at least ⅕ of a second. Only mice whose control reaction time is 10.0 seconds or less are used. At 15, 30, 60 and 120 minutes (±1 to 5 minutes) after test or control article administration, reaction times will be obtained and recorded for the group sequentially.

Analgesic response is an increase in reaction time of the mouse to the heat stimulus. Percent analgesia is calculated from the average response of the group of ten mice per dose level at a specified time interval:

$$\% analgesia = \frac{( average response time in seconds ( test article treated )}{( average response time in seconds ( control )} - 1.0 \times 100$$

An ANOVA with appropriate Multiple Comparison Test is then performed.

Rat Tail Radiant Heat Test (Tail Flick). To evaluate the potential ability of a test article to produce an analgesic response to thermal stimulation in rats.

Following an overnight fast, rats are weighed and placed in groups of ten. The test or vehicle control articles are administered. A Tail Flick Analgesia Meter is used. Sixty minutes following oral administration (or as recommended by the Sponsor), the tail of each rat is exposed to a specific intensity of heat stimulus and the time required to elicit a response (a characteristic tail flick) is recorded.

Percent analgesia will be calculated using the mean control response compared to the mean test article response.

Identification of Compounds for Use as Peripheral Anti-Hyperalgesics. In general, the methods described above, are also useful for assessing peripheral anti-hyperalgesic activities of test compounds. Most preferred among the methods for assessing anti-hyperalgesic activity are those described in Niemegeers et al. (1974) Drug Res. 24:1633-1636.

Assessment of Ratio [C] of the ED₅₀ Value [A] in a Test for Anti-diarrheal Activity, Such as the Castor Oil Test, to the ED₅₀ Value [B] in a Test of CNS Effects, Such as the Tail Withdrawal Test. The agents intended for use in the methods and compositions can be identified by their activity as anti-diarrheals, and their lack of CNS effects. In particular, the selected compound exhibits anti-hyperalgesic activity in any of the standard models, discussed above, and, preferably, either (a) the ratio of these activities [B/A], as measured in standard assays, is substantially greater or equal to [at least equal to, more preferably at least about 2-fold greater] than the ratio of such activities for diphenoxylate; or (b) the activity of the compound in an assay that measures CNS activity is substantially less [at least two-fold, preferably 3-fold or more] than diphenoxylate.

In Vitro Pharmacology cAMP Assay in CHO Cells Expressing Human mu, MOP) Receptor. The mu opioid receptor is Gᵢ coupled, which works by inhibiting a cAMP increase. Thus, changes in cAMP can be used to determine agonist/antagonist activity at the μ receptor. Cellular cAMP may be increased by addition of forskolin. Prior addition of DAMGO, or similar agonists, e.g. endomorphin-1, fentanyl, or morphine, inhibit this forskolin-induced increase. The absence of agonist effect produces a result equivalent to forskolin alone. Therefore, increasing agonist concentration decreases cAMP levels.

Antagonists, such as CTOP, naloxone and ciprodime inhibit the cAMP inhibition. By adding the test compound, then DAMGO, then forskolin, one can determine if the test compound has antagonistic activity. Increasing antagonist concentration increases cAMP.

Extracted cAMP level may be determined via competitive ETA assay utilizing alkaline phosphatase. Additional experimental conditions are as described, for example, in Toll L., J Pharmacol Exp Ther. (1995) 273(2): 721-7.

The disclosures of all patents, patent applications and scientific publications cited or referenced herein are incorporated by reference where appropriate for teachings of additional or alternative details, features, and/or technical background, including U.S. patent application Ser. Nos., 11/441,395 entitled “Synthesis of (R)-N-Methylnaltrexone” and 11/441,452 entitled “(S)-N-Methylnaltrexone” filed May 25, 2006. In case of conflict between documents incorporated by reference and the instant application the instant application will control.

Having thus described several embodiments of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### STATEMENT REGARDING EMBODIMENTS

While the invention has been described with respect to embodiments, those skilled in the art will readily appreciate that various changes and/or modifications can be made to the invention without departing from the spirit or scope of the invention as defined by the appended claims. All documents cited herein are incorporated by reference herein where appropriate for teachings of additional or alternative details, features and/or technical background.

What is claimed is:

1. An isolated compound of the (S) configuration with respect to the nitrogen of Formula I(c): 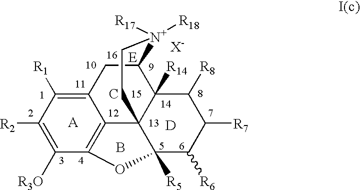
or a pharmaceutically acceptable salt form or prodrug form thereof, wherein:
R₁ and R₂, are independently H, OH, OR₂₆, halide, silyl; hydrocarbyl, cyclohydrocarbyl, or substituted moieties thereof;
or R₁ and R₂ can also be combined to form a C₃-C₆ carbocycle fused ring which may be substituted according to R₁₉, a benzo fused ring, or a 5-6 membered heteroaryl fused ring;
R₃ is H, silyl, CO₂R₁₉, SO₂R₁₉, B(OR₂₆)₂;
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
C₁-C₃ acyl
R₅ is H, OH, OR₂₆,
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
R₆ is H, ═O, OH, OR₂₆, ═(R₁₉)(R_{19′}), =(hetero cycle substituted with 0-3R₂₀), ═(C₃-C₇ cycle substituted with 0-3R₂₀);
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
amine, amide, sulfonamide, or ester;
R₇ and R₈ are independently H, hydrocarbyl, cyclohydrocarbyl, hetero cycle with 0-3R₂₀, alkylaryl with 0-3R₂₀, arylakly with 0-3 R₂₀, or substituted moieties thereof, or 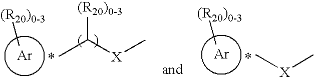
where, X is bond, —O, O, S, N(R₁₉), SO, SO₂, SO₂N(R₁₉), CON(R₁₉), N(R₁₉)CON(R_{19′}), N(R₁₉)C(═NR_{19′})N(R_{19″}), COO;
R₇ and R₈ are combined to form a carbocycle fused ring which may be substituted according to R₁₉, a benzo fused ring, 5-, 6-, or a 5-6 membered aryl or heteroaryl with 0-3R₂₀;
R₁₄ is H, OH, OR₂₆, NR₂₂R₂₃SR₂₅, S(═O)R₂₅, SO₂R₂₅, hetero cycle with 0-3R₂₀, alkylaryl with 0-3R₂₀, arylalkyl with 0-3R₂₀, 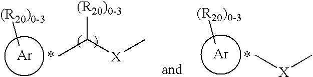
wherein, X is bond, ═O, O, S, N(R₁₉), SO, SO₂, SO₂N(R₁₉), CON(R₁₉), N(R₁₉)CON(R_{19′}), N(R₁₉)C(═NR_{19′})N(R_{19″}), COO;
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀; aryloxy, acyloxy,
or R₁₄ can be combined with R₁₈ depending on its configuration with respect to quaternary nitrogen to form an O-fused ring, or a C₃-C₆ carbocycle fused ring;
R₁₇ and R₁₈ are C₁-C₆ hydrocarbyls which may be substituted, wherein if R₁₈ is methyl, R₁₇ is not allyl, hetero cycle with 0-3R₂₀, alkylaryl with 0-3R₂₀, arylalkyl with 0-3R₂₀. 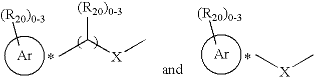
wherein, X is bond, ═O, O, S, N(R₁₉), SO, SO₂, SO₂N(R₁₉), CON(R₁₉), N(R₁₉)CON(R_{19′}), N(R₁₉)C(═NR_{19′})N(R_{19″}), COO;
R₁₉ is at each occurrence is independently selected from: H, C₁-C₆ alkyl, CF₃, OR₂₄, Cl, F, Br, I, ═O, CN, NO₂, NR₂₂R₂₃ aryl substituted with 0-3R₂₁;
C₃-C₁₀ carbocycle substituted with 0-3 R₂₁;
aryl substituted with 0-3 R₂₁; or
5 to 10 membered heterocycle containing 1 to 4 heteroatoms selected from nitrogen, oxygen, and sulphur, wherein said 5 to 10 membered heterocycle is substituted with 0-3 R₂₁;
R₂₀ at each occurrence, is independently selected from H, OH, Cl, F, Br, I, CN, NO₂, NR₂₂R₂₃, acetyl, OR₂₅, XR₂₅,
C₁-C₆ alkyl, C₁-C₄ alkoxy, C₁-C₄ haloalkyl,
C₁-C₄ haloalkoxy, and C₁-C₄ haloalkyl-S—;
R₂₁, at each occurrence, is independently selected from H, OH, Cl, F, Br, I, CN, NO₂, NR₂₂R₂₃, CF₂₃, acetyl, OR₂₄, XR₂₅,
C₁-C₆ alkyl, C₁-C₄ alkoxy, C₁-C₄ haloalkyl,
C₁-C₄ haloalkoxy, and C₁-C₄ haloalkyl-S—; or
NR₂₂R₂₃ may be a heterocyclic ring selected from the group piperidinyl, homopiperidinyl, thiomorpholinyl, piperizinyl, and morpholinyl;
R₂₂, at each occurrence, is independently selected from H, C₁-C₆ alkyl, C₆-C₁₀ aryl, heteroaryl, hetero cycle, alkylaryl, and arylalkyl;
(C₁-C₆ alkyl)-C(═O)—, and (C₁-C₆ alkyl)-S(═O)₂—;
R₂₃, at each occurrence, is independently selected from: H, (C₁-C₆)alkyl, C₆-C₁₀ aryl, hetero aryl, hetero cycle, alkylaryl, haloalkyl, arylalkyl,
(C₁-C₆ alkyl)-C(═O)—, and (C₁-C₆ alkyl)-S(═O)₂—;
R₂₄, at each occurrence, is independently selected from H, phenyl, benzyl, (C₁-C₆) alkyl, and (C₂-C₆) alkoxyalkyl;
R₂₅ is alkyl, aryl, or arylalkyl;
R₂₆ is at each occurrence is independently selected from:
H, C₁-C₆ alkyl, CF₃;
C₃-C₁₀ carbocycle substituted with 0-3 R₂₁;
aryl substituted with 0-3 R₂₁; or
5 to 10 membered heterocycle containing 1 to 4 heteroatoms selected from nitrogen, oxygen, and sulphur, wherein said 5 to 10 membered heterocycle is substituted with 0-3 R₂₁; and
X⁻ is an anion.

2. An isolated compound of the (S) configuration with respect to the nitrogen of formula I: 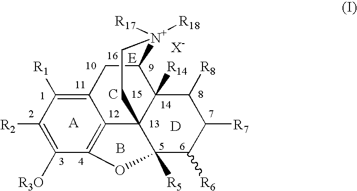
or a pharmaceutically acceptable salt form or prodrug form thereof, wherein:
R₁ and R₂ are independently H, OH, OR₂₆, halide, silyl; hydrocarbyl, cyclohydrocarbyl, or substituted moieties thereof; or R₁ and R₂ can also be combined to form a C₃-C₆ carbocycle fused ring which may be substituted according to R₁₉, a benzo fused ring, or a 5-6 membered heteroaryl fused ring;
R₃ is H, silyl;
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
C₁-C₃ acyl
R₅ is H, OH, OR₂₆,
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
R₆ is H, ═O, OH, OR₂₆;
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀,
amine, amide, sulfonamide, or ester;
R₇ and R₈ are independently H, hydrocarbyl, cyclohydrocarbyl, or substituted moieties thereof; or R₇ and R₅ are combined to form a carbocycle fused ring which may be substituted according to R₁₉, a benzo fused ring, or a 5-6 membered heteroaryl fused ring;
R₁₄ is H, OH, OR₂₆, NR₂₂R₂₃SR₂₅, S(═O)R₂₅, SO₂R₂₅;
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀; aryloxy, acyloxy,
or R₁₄ can be combined with R₁₇ or R₁₈ is depending on its configuration with respect to quaternary nitrogen to form an O-fused ring, or a C₃-C₆ carbocycle fused ring;
R₁₇ and R₁₈ are C₁-C₆ hydrocarbyls which may be substituted, wherein if R₁₈ is methyl, R₁₇ is not allyl;
R₁₉ is at each occurrence is independently selected from:
H, C₁-C₆ alkyl, CF₃, OR₂₄, Cl, F, Br, I, ═O, CN, NO₂, NR₂₂R₂₃;
C₃-C₁₀ carbocycle substituted with 0-3 R₂₁;
aryl substituted with 0-3 R₂₁; or
5 to 10 membered heterocycle containing 1 to 4 heteroatoms selected from nitrogen, oxygen, and sulphur, wherein said 5 to 10 membered heterocycle is substituted with 0-3 R₂₁;
R₂₀ at each occurrence, is independently selected from H, OH, Cl F, Br, I, CN, NO₂, NR₂₂R₂₃, acetyl,
C₁-C₆ alkyl, C₁-C₄ alkoxy, C₁-C₄ haloalkyl,
C₁-C₄ haloalkoxy, and C₁-C₄ haloalkyl-S—;
R₂₁, at each occurrence, is independently selected from H, OH, Cl, F, Br, I, CN, NO₂, NR₂₂R₂₃, CF₃, acetyl,
C₁-C₆ alkyl, C₁-C₄ alkoxy, C₁-C₄ haloalkyl,
C₁-C₄ haloalkoxy, and C₁-C₄ haloalkyl-S—; or
NR₂₂R₂₃ may be a heterocyclic ring selected from the group piperidinyl, homopiperidinyl, thiomorpholinyl, piperizinyl, and morpholinyl;
R₂₂, at each occurrence, is independently selected from H, C₁-C₆ alkyl, (C₁-C₆ alkyl)-C(═O)—, and (C₁-C₆ alkyl)-S(═O)₂—;
R₂₃, at each occurrence, is independently selected from:
H, (C₁-C₆)alkyl,
(C₁-C₆ alkyl)-C(═O)—, and (C₁-C₆ alkyl)-S(═O)₂—;
R₂₄, at each occurrence, is independently selected from H, phenyl, benzyl, (C₁-C₆) alkyl, and (C₂-C₆) alkoxyalkyl;
R₂₅ is alkyl, aryl, or arylalkyl;
R₂₆ is at each occurrence is independently selected from
H, C₁-C₆ alkyl, CF₃;
C₃-C₁₀ carbocycle substituted with 0-3 R₂₁;
aryl substituted with 0-3 R₂₁; or
5 to 10 membered heterocycle containing 1 to 4 heteroatoms selected from nitrogen, oxygen, and sulphur, wherein said 5 to 10 membered heterocycle is substituted with 0-3 R₂₁; and
X⁻ is an anion

3. The compound of Formula I according to claim 2, or a pharmaceutically acceptable salt form or prodrug form thereof, wherein the anion is a halide, sulfate, phosphate, nitrate, or anionic-charged organic species.

4. The compound of Formula (I) according to claim 3, or a pharmaceutically acceptable salt form or prodrug form thereof wherein the halide is bromide.

5. The compound of Formula (I) according to claim 3, or a pharmaceutically acceptable salt form or prodrug form thereof, wherein the halide is iodide.

6. The compound of Formula (I) according to claim 2, or a pharmaceutically acceptable salt form or prodrug form thereof, having at least 90% purity.

7. The compound of Formula (I) according to claim 2, or a pharmaceutically acceptable salt form or prodrug form thereof, having at least 95% purity.

8. The compound of Formula (I) according to claim 2, or a pharmaceutically acceptable salt form or prodrug form thereof, comprising a crystalline form.

9. The compound of Formula (I) according to claim 4, or a pharmaceutically acceptable salt form or prodrug form thereof, comprising a crystalline form.

10. The compound of Formula (I) according to claim 2, or a pharmaceutically acceptable salt form or prodrug form thereof, wherein the S-configuration is 95% pure with respect to the quaternary nitrogen

11. The compound of Formula (I) according to claim 2, or a pharmaceutically acceptable salt form or prodrug form thereof; wherein the S-configuration is 98% pure with respect to the quaternary nitrogen.

12. The compound of Formula (I) according to claim 2, or a pharmaceutically acceptable salt form or prodrug form thereof, wherein the S-configuration is 99.5% pure with respect to the quaternary nitrogen.

13. The compound of Formula (I) according to claim 2, or a pharmaceutically acceptable salt form or prodrug form thereof, wherein the S-configuration is 99.8% pure with respect to the quaternary nitrogen.

14. A composition comprising the compound according claim 2 or a pharmaceutically acceptable salt form or prodrug form thereof, wherein the S-configuration is 99.8% pure with respect to the quaternary nitrogen.

15. The composition of claim 14, wherein the composition is a solution.

16. The composition of claim 14, wherein the composition is a solid.

17. A pharmaceutical composition comprising a therapeutically effective amount of the compound of claim 2, and a pharmaceutically acceptable carrier.

18. The pharmaceutical composition of claim 17 wherein the composition is an oral formulation.

19. The pharmaceutical composition of claim 17 wherein the composition is in a controlled release or sustained release formulation.

20. The pharmaceutical composition of claim 17, wherein the composition is a topical formulation.

21. The pharmaceutical composition of claim 17, wherein the composition is lyophilized.

22. The pharmaceutical composition of claim 17, wherein the composition is a suppository.

23. An inhaler containing the pharmaceutical composition of claim 17.

24. A nasal spray device containing the pharmaceutical composition of claim 17.

25. A pharmaceutical composition of claim 17, further comprising a therapeutic agent other than the compound of compound of claim 2.

26. The pharmaceutical composition of claim 25, wherein the therapeutic agent is an opioid agonist.

27. The pharmaceutical composition of claim 26, wherein the opioid is selected from the group consisting of alfentanil, anileridine, asimadoline, bremazocine, burprenorphine, butorphanol, codeine, dezocine, diacetylmorphine (heroin), saturatedcodeine, diphenoxylate, fedotozine, fentanyl, funaltrexamine, hydrocodone, hydromorphone, levallorphan, levomethadyl acetate, levorphanol, loperamide, meperidine (pethidine), methadone, morphine, morphine-6-glucuronide, nalbuphine, nalorphine, opium, oxycodone, oxymorphone, pentazocine, propiram, propoxyphene, remifentanyl, sufentanil, tilidine, trimebutine, tramadol, and combinations thereof.

28. The pharmaceutical composition of claim 26, wherein the opioid or opioid agonist has substantially no central nervous system (CNS) activity.

29. The pharmaceutical composition of claim 25, wherein the therapeutic agent is not an opioid, opioid agonist or an opioid antagonist.

30. The pharmaceutical composition of claim 29, wherein the therapeutic agent is an non-opioid analgesic/anti-oyretic, antiviral agent, an antibiotic agent, an antifungal agent, antibacterial agent, antiseptic agent, anti-protozoal agent, anti-parasitic agent, an anti-inflammatory agent, a vasoconstrictor agent, a local anesthetic agent, an anti-diarrheal agent, an anti-hyperalgesia agent, or combinations thereof.

31. The pharmaceutical composition of claim 29, wherein the therapeutic agent is an anti-diarrhea agent that is loperamide, loperamide analogs, N-oxides of loperamide and analogs, metabolites and prodrugs thereof, diphenoxylate, cisapride, antacids, aluminum hydroxide, magnesium aluminum silicate, magnesium carbonate, magnesium hydroxide, calcium carbonate, polycarbophil, simethicone, hyoscyamine, atropine, furazolidone, difenoxin, octreotide, lansoprazole, kaolin, pectin, activated charcoal, sulphaguanidine, succinylsulphathiazole, phthalylsulphathiazole, bismuth aluminate, bismuth subcarbonate, bismuth subcitrate, bismuth citrate, tripotassium dicitrato bismuthate, bismuth tartrate, bismuth subsalicylate, bismuth subnitrate and bismuth subgallate, opium tincture (paregoric), herbal medicines, plant-derived anti-diarrheal agents or combinations thereof.

32. The pharmaceutical composition of claim 29, wherein the therapeutic agent is an anti-inflammatory agent that is a non-steroidal anti-inflammatory drug (NSAID), a tumor necrosis factor inhibitor, basiliximab, daclizumab, infliximab, mycophenolate, mofetil, azothioprine, tacrolimus, steroids, sulfasalazine, olsalazine, mesalamine, or combinations thereof.

33. The pharmaceutical composition of claim 29, wherein the therapeutic agent is an anti-viral agent.

34. The pharmaceutical composition of claim 29, wherein the therapeutic agent is an anti-bacterial agent.

35. The pharmaceutical composition of claim 29, wherein the therapeutic agent is an anti-hyperalgesia agent.

36. A method for inhibiting diarrhea in a subject comprising administering to a subject in need of such treatment the pharmaceutical composition of claim 17 in an amount effective to treat or prevent the diarrhea.

37. The method of claim 36, further comprising administering to the subject an anti-diarrhea agent that is not (S)-N-stereoisomer of the compound of claim F.

38. The method of claim 37, wherein the anti-diarrhea agent that is not the (S)-N-stereoisomer of the compound of claim 2 is an opioid or an opioid agonist.

39. A method of reducing a volume of discharge from a ileostomy or colostomy in a subject comprising administering to the subject in need of such reduction the pharmaceutical composition of claim 17 in an amount effective to reduce the volume of discharge from the ileostomy or colostomy.

40. A method of reducing a rate of discharge from a ileostomy or colostomy in a subject comprising administering to the subject in need of such reduction the pharmaceutical composition of claim 17 in an amount effective to reduce the rate of discharge from the ileostomy or colostomy.

41. A method for inhibiting gastrointestinal motility in a subject in need of such treatment comprising administering to the subject a pharmaceutical composition of claim 17 in an amount effective to inhibit gastrointestinal motility in the subject.

42. The method of claim 41 further comprising administering to the subject an opioid or an opioid agonist.

43. A method for treating irritable bowel syndrome comprising administering to a patient in need of such treatment the pharmaceutical composition of claim 17, in an amount effective to ameliorate at least one symptom of the irritable bowel syndrome.

44. A method for inhibiting pain in a subject comprising administering the pharmaceutical composition of claim 17 in an amount sufficient to prevent or treat the pain.

45. The method of claim 44 further comprising administering to the subject a therapeutic agent other than the (S)-N-stereoisomer of the compound of claim 2 in the composition.

46. The method of claim 45, wherein the therapeutic agent other than (S)-N-stereoisomer of the compound of claim 2 in the composition is an opioid.

47. The method of claim 45, wherein the therapeutic agent other than (S)-N-stereoisomer of the compound of claim 2 in the composition is an antiviral agent, an antibiotic agent, an antifungal agent, antibacterial agent, antiseptic agent, anti-protozoal agent, anti-parasitic agent, an anti-inflammatory agent, a vasoconstrictor agent, a local anesthetic agent, an anti-diarrheal agent, or an anti-hyperalgesia agent.

48. The method of claim 44, wherein the pain is peripheral hyperalgesia.

49. The method of claim 44, wherein the pharmaceutical composition is administered locally to a site of the pain.

50. The method of claim 44, wherein the administration is intra-articular.

51. The method of claim 44, wherein the administration is systemic.

52. The method of claim 44, wherein the administration is topical.

53. The method of claim 44, wherein the composition is administered to the eye.

54. A method for inhibiting inflammation in a subject comprising administering to a subject in need thereof the pharmaceutical composition of claim 17 in an amount effective to inhibit the inflammation.

55. The method of claim 54 further comprising administering to the subject a therapeutic agent other than (S)-N-stereoisomer of the compound of claim 2 in the composition.

56. The method of claim 55 wherein the therapeutic agent other than (S)-N-stereoisomer of the compound of claim 2 is an anti-inflammatory agent.

57. A method of inhibiting production of tumor necrosis factor (TNF) in a subject, comprising administering to the subject a composition comprising TNF production-inhibitory amount of a pharmaceutical composition of claim 17.

58. A kit comprising a package containing a sealed container comprising the pharmaceutical composition of claim 17 and instructions for use.

59. The kit according to claim 58, further comprising a combination of compatible therapeutic agents wherein one of the therapeutic agents is a peripheral opioid antagonist.

60. The method of claim 17, wherein the peripheral opioid antagonist is the counterpart (R)-N-stereoisomer of the compound.

61. A composition comprising an (S) compound of claim 2, wherein the composition is free of HPLC detectable counterpart (R)-stereoisomer at a detection limit of 0.02% and at a quantitation limit of 0.05%.

62. An isolated compound of the (S) configuration with respect to the nitrogen of formula Ia: 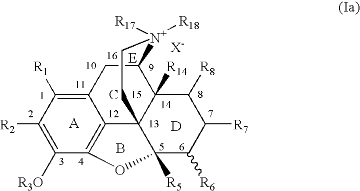
wherein
R₁₇ and R₁₈ are selected alternatively with respect to one another from (a) or (b):
(a) unsubstituted or non-halogen substituted: C₄-C₈ (cycloalkyl)alkyl or (cycloalkenyl)alkyl, (cycloheteryl)alkyl, (cycloaryl)alkyl; C₄-C₆ (cycloalkyl)alkyl or (cycloalkenyl)alkyl, (cycloheteryl)alkyl, (cycloaryl)alkyl
(b) substituted or unsubstituted linear or branched C₁-C₃ alkyl, C₂-C₃ alkenyl, or C₃-alkynyl;
wherein if (b) is selected as methyl, and R₆ is ═O, (a) is not unsubstituted (cyclopropyl)methyl;
R₆ is H, OH, ═O, ═CH₂, —N(CH₃)₂, or any cyclic ring, or forms a cyclic ring with R₇;
R₇ and R₈ are H or alkyl;
R₁₄ is H, OH, halide, arylamido, amino, N-alkyl, N-dialkyl, N-aryl, N-alkylaryl, N-cycloalkylalkyl, SCH₃, S(═O)CH₃, S(═O)₂CH₃, alkoxy, aryloxy, or aryl-alkoxy or forms a cyclic ring with R₁₇ or R₁₈;
R₁ and R₂ are independently H, halide, alkoxy, alkyl, or aryl;
R₃ is H, C₁-C₄ alkyl, or C₁-C₃ acyl, -silyl;
R₅ is H, OH, alkyl, alkoxy, or aryloxy; and
X⁻ is an anion.

63. An isolated compound of the (S) configuration with respect to the nitrogen of Formula Ib: 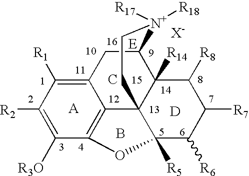
wherein
R₁₇ and R₁₈ are a substituted or unsubstituted C₁-C₆ hydrocarbyl, wherein when R₆ is selected as ═O, at least one of which is not methyl when the other is cyclopropylmethyl;
R₆ is H, OH, OR₂₅, ═O, ═CH₂, —N-alkyl, N-dialkyl, acyloxy, alkoxy, alkyl, ═CR′R″ where R′ and R″ are independently H or C₁-C₁₀ alkyl, or any ring, or R₆ forms a ring with R₇;
R₇ and R₅ are H or hydrocarbyl, cyclohydrocarbyl, alkoxy, amine, amide, hydroxy or substituted moieties thereof;
R₁₄ is H, OH, halide, N-alkyl, N-dialkyl, N-aryl, N-alkylaryl, N-cycloalkylalkyl, SR₂₅, S(═O)R₂₅, SO₂R₂₅; alkoxy, aryloxy, or arylalkoxy, or forms a ring with R₁₇ or R₁₈;
R₁ and R₂ are independently H, halide, alkoxy, alkyl, or aryl;
R₃ is H, alkyl, C₁-C₃ acyl, silyl;
R₅ is H, OH, alkyl, alkoxy, or aryloxy;
R₂₅ is alkyl, aryl, arylalkyl; and
X⁻ is an anion.
